(19) United States
(12) Patent Application Publication
(10) Pub. No.: US 2026/0098096 A1
(43) Pub. Date: Apr. 9, 2026

(54) ANTI-CCR8 ANTIBODIES

(71) Applicant: MONASH UNIVERSITY, Clayton, Victoria (AU)

(72) Inventor: Remy Michel ROBERT, Clayton, Victoria (AU)

(21) Appl. No.: 19/113,897

(22) PCT Filed: Sep. 21, 2023
(86) PCT No.: PCT/AU2023/050916

§ 371 Date: Mar. 20, 2025

(30) Foreign Application Priority Data
Sep. 21, 2022 (AU) 2022902742

(51) Int. Cl. C07K 16/28; A61K 39/00; A61P 35/00

(52) U.S. Cl. CPC C07K 16/2866; A61P 35/00; A61K 2039/505; C07K 2317/24; C07K 2317/31; C07K 2317/524; C07K 2317/526; C07K 2317/565; C07K 2317/567; C07K 2317/622; C07K 2317/732

(57) ABSTRACT

The invention relates to antigen binding proteins and related fragments thereof for binding to CCR8, to production of said antigen binding proteins and fragments and to use of said antigen binding proteins and fragments for detection and therapy of various conditions.

### FIELD OF THE INVENTION

The invention relates to antigen binding proteins and related fragments thereof for binding to CCR8, to production of said antigen binding proteins and fragments and to use of said antigen binding proteins and fragments for detection and therapy of various conditions.

### RELATED APPLICATION

This application claims priority from Australian provisional application 2022902742, the entire contents of which is incorporated herein by reference.

### BACKGROUND OF THE INVENTION

Immunotherapy is a rapidly advancing and very promising treatment for various forms of cancer, with many recent successes. However, some patients have limited to no response to current immunotherapies, and others exhibit relapse following initial responsiveness.

The human immune system includes checks and balances that serve to stop an overactive immune system from harming the body. Regulatory T cells (“Tregs”) have a vital role in maintaining a functional immune system by suppressing the immune response. In particular, Tregs are a major immune cell population that plays a crucial role in maintaining self-tolerance and resolution of immune responses by employing multifaceted immunoregulatory mechanisms. However, Tregs readily infiltrate into the tumor microenvironment (TME) and dampen anti-tumor immune responses, thereby becoming a barrier to effective cancer immunotherapy. Treg modulation strategies have been shown to increase antitumor immunity and reduce tumor burden in both preclinical and clinical settings.

C-C chemokine receptor 8 (CCR8) is a chemokine receptor that is selectively expressed on a subset of intratumoral Tregs bearing the highest levels of suppressive markers, and its expression correlates with poor prognosis in multiple tumor types. This subset of Tregs expressing CCR8 (CCR8+ Tregs) has been demonstrated to be a major driver of immunosuppression and is critical for Treg function and suppression.

Accordingly, there remains a need in the art to develop CCR8-targeted therapeutics, such as anti-CCR8 antibodies, that can be used for therapeutic purposes in the treatment of cancer.

Reference to any prior art in the specification is not an acknowledgment or suggestion that this prior art forms part of the common general knowledge in any jurisdiction or that this prior art could reasonably be expected to be understood, regarded as relevant, and/or combined with other pieces of prior art by a skilled person in the art.

### SUMMARY OF THE INVENTION

In one aspect, the present invention provides an antigen binding protein that binds to or specifically binds to the extracellular loop 2 of CCR8, preferably wherein the antigen binding protein does not block binding of CCR8 to CCL1.

In any embodiment, the antigen binding protein binds to residues 172 to 202 of CCR8, wherein the amino acid sequence of CCR8 is as set forth in SEQ ID NO: 55. In any embodiment, the antigen binding protein binds to residues 172 to 190 of CCR8, wherein the amino acid sequence of CCR8 is as set forth in SEQ ID NO: 55. In any embodiment, the antigen binding protein bind to residues 176 to 179 of CCR8, wherein the amino acid sequence of CCR8 is as set forth in SEQ ID NO: 55.

In one aspect, the present invention provides an antigen binding protein comprising a CDRH1, a CDRH2 and/or a CDRH3 of an antibody having a variable heavy chain as defined in SEQ ID NO: 1.

In another aspect, the invention provides an antigen binding protein comprising a CDRL1, a CDRL2 and/or a CDRL3 of an antibody having a variable light chain as defined in SEQ ID NO: 2.

In another aspect, the invention provides an antigen binding protein comprising a CDRH1, a CDRH2 and/or a CDRH3 of an antibody having a variable heavy chain as defined in SEQ ID NO: 1 and a CDRL1, a CDRL2 and/or a CDRL3 of an antibody having a variable light chain as defined in SEQ ID NO: 2.

In another aspect, the invention provides an antigen binding protein for binding to CCR8, the antigen binding protein comprising:

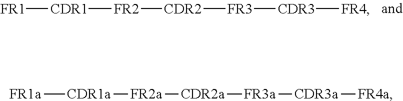

wherein:
FR1, FR2, FR3 and FR4 are each framework regions;
CDR1, CDR2 and CDR3 are each complementarity determining regions;
FR1a, FR2a, FR3a and FR4a are each framework regions;
CDR1a, CDR2a and CDR3a are each complementarity determining regions;
   wherein the sequence of any of the framework regions or complementarity determining regions are as described herein.

In another aspect, the invention provides an antigen binding protein for binding to CCR8, the antigen binding protein comprising:

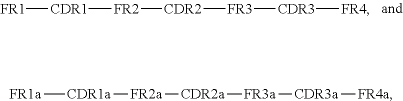

wherein:
FR1, FR2, FR3 and FR4 are each framework regions;
CDR1, CDR2 and CDR3 are each complementarity determining regions;
FR1a, FR2a, FR3a and FR4a are each framework regions;
CDR1a, CDR2a and CDR3a are each complementarity determining regions;
   wherein the sequence of any of the complementarity determining regions have an amino acid sequence as described in Table 1 below. Preferably, the framework regions have an amino acid sequence also as described in Table 1 below, including amino acid variation at particular residues which can be determined by aligning the various framework regions derived from each antibody. The invention also includes where CDR1, CDR2 and CDR3 are sequences from the variable heavy chain of an antibody (a VH), CDR1a, CDR2a and CDR3a are sequences from the variable light chain of an antibody (a VL), or where CDR1, CDR2 and CDR3 are sequences from a VL, CDR1a, CDR2a and CDR3a are sequences from a VH.

In any embodiment, an antigen binding protein described herein comprises:

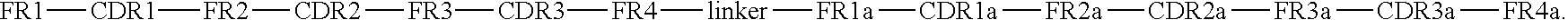

As defined herein, the linker may be a chemical, one or more amino acids, or a disulphide bond formed between two cysteine residues.

In certain preferred embodiments, the invention provides an antigen binding protein comprising, consisting essentially of or consisting of the amino acid sequence of (in order of N to C terminus or C to N terminus) SEQ ID NOs: 1 and 2.

Optionally the antigen binding protein comprises SEQ ID NO: 2 (VL)-linker-SEQ ID NO: 1 (VH), or comprises SEQ ID NO: 1 (VH)-linker-SEQ ID NO: 2 (VL).

In certain preferred embodiments, the invention provides an antigen binding protein comprising, consisting essentially of or consisting of the amino acid sequence of (in order of N to C terminus or C to N terminus) SEQ ID NOs: 83 and 84.

Optionally the antigen binding protein comprises SEQ ID NO: 84 (VL)-linker-SEQ ID NO: 83 (VH), or comprises SEQ ID NO: 83 (VH)-linker-SEQ ID NO: 84 (VL).

In any embodiment, the invention provides an antigen binding protein that binds to or specifically binds to CCR8 and wherein the antigen binding protein competitively inhibits the binding of an antibody comprising a VH comprising a sequence as set forth in SEQ ID NO: 1 and a VL comprising a sequence as set forth in SEQ ID NO: 2.

In any embodiment, the invention provides an antigen binding protein that binds to or specifically binds to CCR8 and wherein the antigen binding protein competitively inhibits the binding of an antibody comprising a VH comprising a sequence as set forth in SEQ ID NO: 83 and a VL comprising a sequence as set forth in SEQ ID NO: 84.

In one aspect, the present invention also provides an antigen binding protein comprising an antigen binding domain of an antibody, wherein the antigen binding domain binds to or specifically binds to CCR8, wherein the antigen binding domain comprises at least one of:
(i) a VH comprising a complementarity determining region (CDR) 1 comprising a sequence at least about 80%, at least 85%, at least 90%, at least 91% at least 92%, at least 93%, at least 94%, at least 95%, at least 96%, at least 97%, at least 98%, at least 99% identical to a sequence set forth in SEQ ID NO: 5, a CDR2 comprising a sequence at least about 80%, at least 85%, at least 90%, at least 92%, at least 95%, at least 97%, at least 99% identical to a sequence set in SEQ ID NO: 6, and a CDR3 comprising a sequence at least about 80%, at least 85%, at least 90%, at least 92%, at least 95%, at least 97%, at least 99% identical to a sequence set forth in SEQ ID NO: 7;
(ii) a VH comprising a sequence at least about 80%, at least 85%, at least 90%, at least 91% at least 92%, at least 93%, at least 94%, at least 95%, at least 96%, at least 97%, at least 98%, at least 99% identical to a sequence set forth in SEQ ID NO: 1;
(iii) a VL comprising a CDR1 comprising a sequence at least about 80%, at least 85%, at least 90%, at least 91% at least 92%, at least 93%, at least 94%, at least 95%, at least 96%, at least 97%, at least 98%, at least 99% identical to a sequence set forth in SEQ ID NO: 8, a CDR2 comprising a sequence at least about 80%, at least 85%, at least 90%, at least 91% at least 92%, at least 93%, at least 94%, at least 95%, at least 96%, at least 97%, at least 98%, at least 99% identical to a sequence set forth in SEQ ID NO: 9, and a CDR3 comprising a sequence at least about 80%, at least 85%, at least 90%, at least 91% at least 92%, at least 93%, at least 94%, at least 95%, at least 96%, at least 97%, at least 98%, at least 99% identical to a sequence set forth in SEQ ID NO: 10;
(iv) a VL comprising a sequence at least about 80%, at least 85%, at least 90%, at least 91% at least 92%, at least 93%, at least 94%, at least 95%, at least 96%, at least 97%, at least 98%, at least 99% identical to a sequence set forth in SEQ ID NO: 2;
(v) a VH comprising a CDR1 comprising a sequence set forth in SEQ ID NO: 5, a CDR2 comprising a sequence set forth between in SEQ ID NO: 6, and a CDR3 comprising a sequence set forth in SEQ ID NO: 7;
(vi) a VH comprising a sequence set forth in SEQ ID NO: 1;
(vii) a VL comprising a CDR1 comprising a sequence set SEQ ID NO: 8, a CDR2 comprising a sequence set forth in SEQ ID NO: 9 and a CDR3 comprising a sequence set forth in SEQ ID NO: 10;
(viii) a VL comprising a sequence set forth in SEQ ID NO: 2;
(ix) a VH comprising a CDR1 comprising a sequence set forth in SEQ ID NO: 5, a CDR2 comprising a sequence set forth between in SEQ ID NO: 6 and a CDR3 comprising a sequence set forth in SEQ ID NO: 7; and a VL comprising a CDR1 comprising a sequence set SEQ ID NO: 8, a CDR2 comprising a sequence set forth in SEQ ID NO: 9 and a CDR3 comprising a sequence set forth in SEQ ID NO: 10; or
(x) a VH comprising a sequence set forth in SEQ ID NO: 1 and a VL comprising a sequence set forth in SEQ ID NO: 2.

In one aspect, the present invention also provides an antigen binding protein comprising an antigen binding domain of an antibody, wherein the antigen binding domain binds to or specifically binds to CCR8, wherein the antigen binding domain comprises at least one of:
(i) a VH comprising a complementarity determining region (CDR) 1 comprising a sequence at least about 80%, at least 85%, at least 90%, at least 91% at least 92%, at least 93%, at least 94%, at least 95%, at least 96%, at least 97%, at least 98%, at least 99% identical to a sequence set forth in SEQ ID NO: 5, a CDR2 comprising a sequence at least about 80%, at least 85%, at least 90%, at least 92%, at least 95%, at least 97%, at least 99% identical to a sequence set in SEQ ID NO: 6, and a CDR3 comprising a sequence at least about 80%, at least 85%, at least 90%, at least 92%, at least 95%, at least 97%, at least 99% identical to a sequence set forth in SEQ ID NO: 7;
(ii) a VH comprising a sequence at least about 80%, at least 85%, at least 90%, at least 91% at least 92%, at least 93%, at least 94%, at least 95%, at least 96%, at least 97%, at least 98%, at least 99% identical to a sequence set forth in SEQ ID NO: 83;
(iii) a VL comprising a CDR1 comprising a sequence at least about 80%, at least 85%, at least 90%, at least 91% at least 92%, at least 93%, at least 94%, at least 95%, at least 96%, at least 97%, at least 98%, at least 99% identical to a sequence set forth in SEQ ID NO: 8, a CDR2 comprising a sequence at least about 80%, at least 85%, at least 90%, at least 91% at least 92%, at least 93%, at least 94%, at least 95%, at least 96%, at least 97%, at least 98%, at least 99% identical to a sequence set forth in SEQ ID NO: 9, and a CDR3 comprising a sequence at least about 80%, at least 85%, at least 90%, at least 91% at least 92%, at least 93%, at least 94%, at least 95%, at least 96%, at least 97%, at least 98%, at least 99% identical to a sequence set forth in SEQ ID NO: 10;
(iv) a VL comprising a sequence at least about 80%, at least 85%, at least 90%, at least 91% at least 92%, at least 93%, at least 94%, at least 95%, at least 96%, at least 97%, at least 98%, at least 99% identical to a sequence set forth in SEQ ID NO: 84;
(v) a VH comprising a CDR1 comprising a sequence set forth in SEQ ID NO: 5, a CDR2 comprising a sequence set forth between in SEQ ID NO: 6, and a CDR3 comprising a sequence set forth in SEQ ID NO: 7;
(vi) a VH comprising a sequence set forth in SEQ ID NO: 83;
(vii) a VL comprising a CDR1 comprising a sequence set SEQ ID NO: 8, a CDR2 comprising a sequence set forth in SEQ ID NO: 9 and a CDR3 comprising a sequence set forth in SEQ ID NO: 10;
(viii) a VL comprising a sequence set forth in SEQ ID NO: 84;
(ix) a VH comprising a CDR1 comprising a sequence set forth in SEQ ID NO: 5, a CDR2 comprising a sequence set forth between in SEQ ID NO: 6 and a CDR3 comprising a sequence set forth in SEQ ID NO: 7; and a VL comprising a CDR1 comprising a sequence set SEQ ID NO: 8, a CDR2 comprising a sequence set forth in SEQ ID NO: 9 and a CDR3 comprising a sequence set forth in SEQ ID NO: 10; or
(x) a VH comprising a sequence set forth in SEQ ID NO: 83 and a VL comprising a sequence set forth in SEQ ID NO: 84.

In any aspect of the invention, the antigen binding domain further comprises at least one of:
(i) a VH comprising a framework region (FR) 1 comprising a sequence at least about 80%, at least 85%, at least 90%, at least 91% at least 92%, at least 93%, at least 94%, at least 95%, at least 96%, at least 97%, at least 98%, at least 99% identical to a sequence set forth in SEQ ID NO: 11, a FR2 comprising a sequence at least about 80%, at least 85%, at least 90%, at least 91% at least 92%, at least 93%, at least 94%, at least 95%, at least 96%, at least 97%, at least 98%, at least 99% identical to a sequence set in SEQ ID NO: 12, a FR3 comprising a sequence at least about 80%, at least 85%, at least 90%, at least 91% at least 92%, at least 93%, at least 94%, at least 95%, at least 96%, at least 97%, at least 98%, at least 99% identical to a sequence set forth in SEQ ID NO: 13, and a FR4 comprising a sequence at least about 80%, at least 85%, at least 90%, at least 91% at least 92%, at least 93%, at least 94%, at least 95%, at least 96%, at least 97%, at least 98%, at least 99% identical to a sequence set forth in SEQ ID NO: 14;
(ii) a VL comprising a FR1 comprising a sequence at least about 80%, at least 85%, at least 90%, at least 91% at least 92%, at least 93%, at least 94%, at least 95%, at least 96%, at least 97%, at least 98%, at least 99% identical to a sequence set forth in SEQ ID NO: 15, a FR2 comprising a sequence at least about 80%, at least 85%, at least 90%, at least 91% at least 92%, at least 93%, at least 94%, at least 95%, at least 96%, at least 97%, at least 98%, at least 99% identical to a sequence set forth in SEQ ID NO: 16, a FR3 comprising a sequence at least about 80%, at least 85%, at least 90%, at least 91% at least 92%, at least 93%, at least 94%, at least 95%, at least 96%, at least 97%, at least 98%, at least 99% identical to a sequence set forth in SEQ ID NO: 17, and a FR4 comprising a sequence at least about 80%, at least 85%, at least 90%, at least 91% at least 92%, at least 93%, at least 94%, at least 95%, at least 96%, at least 97%, at least 98%, at least 99% identical to a sequence set forth in SEQ ID NO: 18;
(iii) a VH comprising a FR1 comprising a sequence set forth in SEQ ID NO: 11, a FR2 comprising a sequence set forth between in SEQ ID NO: 12, a FR3 comprising a sequence set forth in SEQ ID NO: 13, and a FR4 comprising a sequence set forth in SEQ ID NO: 14;
(iv) a VL comprising a FR1 comprising a sequence set forth in SEQ ID NO: 15, a FR2 comprising a sequence set forth between in SEQ ID NO: 16, a FR3 comprising a sequence set forth in SEQ ID NO: 17, and a FR4 comprising a sequence set forth in SEQ ID NO: 18; or
(v) a VH comprising a FR1 comprising a sequence set forth in SEQ ID NO: 11, a FR2 comprising a sequence set forth between in SEQ ID NO: 12, a FR3 comprising a sequence set forth in SEQ ID NO: 13, and a FR4 comprising a sequence set forth in SEQ ID NO: 14; and a VL comprising a FR1 comprising a sequence set forth in SEQ ID NO: 15, a FR2 comprising a sequence set forth between in SEQ ID NO: 16, a FR3 comprising a sequence set forth in SEQ ID NO: 17, and a FR4 comprising a sequence set forth in SEQ ID NO: 18.

In any aspect of the invention, the antigen binding domain further comprises at least one of:
(i) a VH comprising a framework region (FR) 1 comprising a sequence at least about 80%, at least 85%, at least 90%, at least 91% at least 92%, at least 93%, at least 94%, at least 95%, at least 96%, at least 97%, at least 98%, at least 99% identical to a sequence set forth in SEQ ID NO: 60, a FR2 comprising a sequence at least about 80%, at least 85%, at least 90%, at least 91% at least 92%, at least 93%, at least 94%, at least 95%, at least 96%, at least 97%, at least 98%, at least 99% identical to a sequence set in SEQ ID NO: 61, a FR3 comprising a sequence at least about 80%, at least 85%, at least 90%, at least 91% at least 92%, at least 93%, at least 94%, at least 95%, at least 96%, at least 97%, at least 98%, at least 99% identical to a sequence set forth in SEQ ID NO: 62, and a FR4 comprising a sequence at least about 80%, at least 85%, at least 90%, at least 91% at least 92%, at least 93%, at least 94%, at least 95%, at least 96%, at least 97%, at least 98%, at least 99% identical to a sequence set forth in SEQ ID NO: 63;
(ii) a VL comprising a FR1 comprising a sequence at least about 80%, at least 85%, at least 90%, at least 91% at least 92%, at least 93%, at least 94%, at least 95%, at least 96%, at least 97%, at least 98%, at least 99% identical to a sequence set forth in SEQ ID NO: 64, a FR2 comprising a sequence at least about 80%, at least 85%, at least 90%, at least 91% at least 92%, at least 93%, at least 94%, at least 95%, at least 96%, at least 97%, at least 98%, at least 99% identical to a sequence set forth in SEQ ID NO: 65, a FR3 comprising a sequence at least about 80%, at least 85%, at least 90%, at least 91% at least 92%, at least 93%, at least 94%, at least 95%, at least 96%, at least 97%, at least 98%, at least 99% identical to a sequence set forth in SEQ ID NO: 66, and a FR4 comprising a sequence at least about 80%, at least 85%, at least 90%, at least 91% at least 92%, at least 93%, at least 94%, at least 95%, at least 96%, at least 97%, at least 98%, at least 99% identical to a sequence set forth in SEQ ID NO: 67;
(iii) a VH comprising a FR1 comprising a sequence set forth in SEQ ID NO: 60, a FR2 comprising a sequence set forth between in SEQ ID NO: 61, a FR3 comprising a sequence set forth in SEQ ID NO: 62, and a FR4 comprising a sequence set forth in SEQ ID NO: 63;
(iv) a VL comprising a FR1 comprising a sequence set forth in SEQ ID NO: 64, a FR2 comprising a sequence set forth between in SEQ ID NO: 65, a FR3 comprising a sequence set forth in SEQ ID NO: 66, and a FR4 comprising a sequence set forth in SEQ ID NO: 67; or
(v) a VH comprising a FR1 comprising a sequence set forth in SEQ ID NO: 60, a FR2 comprising a sequence set forth between in SEQ ID NO: 61, a FR3 comprising a sequence set forth in SEQ ID NO: 62, and a FR4 comprising a sequence set forth in SEQ ID NO: 63; and a VL comprising a FR1 comprising a sequence set forth in SEQ ID NO: 64, a FR2 comprising a sequence set forth between in SEQ ID NO: 65, a FR3 comprising a sequence set forth in SEQ ID NO: 66, and a FR4 comprising a sequence set forth in SEQ ID NO: 67.

In one aspect, the present invention also provides an antigen binding protein comprising an antigen binding domain of an antibody, wherein the antigen binding domain binds to or specifically binds to CCR8, wherein the antigen binding domain comprises at least one of:
(i) a VH comprising a complementarity determining region (CDR) 1 comprising a sequence at least about 80%, at least 85%, at least 90%, at least 91% at least 92%, at least 93%, at least 94%, at least 95%, at least 96%, at least 97%, at least 98%, at least 99% identical to a sequence set forth in SEQ ID NO: 19, a CDR2 comprising a sequence at least about 80%, at least 85%, at least 90%, at least 92%, at least 95%, at least 97%, at least 99% identical to a sequence set in SEQ ID NO: 20, and a CDR3 comprising a sequence at least about 80%, at least 85%, at least 90%, at least 92%, at least 95%, at least 97%, at least 99% identical to a sequence set forth in SEQ ID NO: 21;
(ii) a VH comprising a sequence at least about 80%, at least 85%, at least 90%, at least 91% at least 92%, at least 93%, at least 94%, at least 95%, at least 96%, at least 97%, at least 98%, at least 99% identical to a sequence set forth in SEQ ID NO: 1;
(iii) a VL comprising a CDR1 comprising a sequence at least about 80%, at least 85%, at least 90%, at least 91% at least 92%, at least 93%, at least 94%, at least 95%, at least 96%, at least 97%, at least 98%, at least 99% identical to a sequence set forth in SEQ ID NO: 22, a CDR2 comprising a sequence at least about 80%, at least 85%, at least 90%, at least 91% at least 92%, at least 93%, at least 94%, at least 95%, at least 96%, at least 97%, at least 98%, at least 99% identical to a sequence set forth in SEQ ID NO: 23, and a CDR3 comprising a sequence at least about 80%, at least 85%, at least 90%, at least 91% at least 92%, at least 93%, at least 94%, at least 95%, at least 96%, at least 97%, at least 98%, at least 99% identical to a sequence set forth in SEQ ID NO: 10;
(iv) a VL comprising a sequence at least about 80%, at least 85%, at least 90%, at least 91% at least 92%, at least 93%, at least 94%, at least 95%, at least 96%, at least 97%, at least 98%, at least 99% identical to a sequence set forth in SEQ ID NO: 2;
(v) a VH comprising a CDR1 comprising a sequence set forth in SEQ ID NO: 19, a CDR2 comprising a sequence set forth between in SEQ ID NO: 20, and a CDR3 comprising a sequence set forth in SEQ ID NO: 21;
(vi) a VH comprising a sequence set forth in SEQ ID NO: 1;
(vii) a VL comprising a CDR1 comprising a sequence set SEQ ID NO: 22, a CDR2 comprising a sequence set forth in SEQ ID NO: 23 and a CDR3 comprising a sequence set forth in SEQ ID NO: 10;
(viii) a VL comprising a sequence set forth in SEQ ID NO: 2;
(ix) a VH comprising a CDR1 comprising a sequence set forth in SEQ ID NO: 19, a CDR2 comprising a sequence set forth between in SEQ ID NO: 20 and a CDR3 comprising a sequence set forth in SEQ ID NO: 21; and a VL comprising a CDR1 comprising a sequence set SEQ ID NO: 22, a CDR2 comprising a sequence set forth in SEQ ID NO: 23 and a CDR3 comprising a sequence set forth in SEQ ID NO: 10; or
(x) a VH comprising a sequence set forth in SEQ ID NO: 1 and a VL comprising a sequence set forth in SEQ ID NO: 2.

In one aspect, the present invention also provides an antigen binding protein comprising an antigen binding domain of an antibody, wherein the antigen binding domain binds to or specifically binds to CCR8, wherein the antigen binding domain comprises at least one of:
(i) a VH comprising a complementarity determining region (CDR) 1 comprising a sequence at least about 80%, at least 85%, at least 90%, at least 91% at least 92%, at least 93%, at least 94%, at least 95%, at least 96%, at least 97%, at least 98%, at least 99% identical to a sequence set forth in SEQ ID NO: 19, a CDR2 comprising a sequence at least about 80%, at least 85%, at least 90%, at least 92%, at least 95%, at least 97%, at least 99% identical to a sequence set in SEQ ID NO: 20, and a CDR3 comprising a sequence at least about 80%, at least 85%, at least 90%, at least 92%, at least 95%, at least 97%, at least 99% identical to a sequence set forth in SEQ ID NO: 21;
(ii) a VH comprising a sequence at least about 80%, at least 85%, at least 90%, at least 91% at least 92%, at least 93%, at least 94%, at least 95%, at least 96%, at least 97%, at least 98%, at least 99% identical to a sequence set forth in SEQ ID NO: 83;
(iii) a VL comprising a CDR1 comprising a sequence at least about 80%, at least 85%, at least 90%, at least 91% at least 92%, at least 93%, at least 94%, at least 95%, at least 96%, at least 97%, at least 98%, at least 99% identical to a sequence set forth in SEQ ID NO: 22, a CDR2 comprising a sequence at least about 80%, at least 85%, at least 90%, at least 91% at least 92%, at least 93%, at least 94%, at least 95%, at least 96%, at least 97%, at least 98%, at least 99% identical to a sequence set forth in SEQ ID NO: 23, and a CDR3 comprising a sequence at least about 80%, at least 85%, at least 90%, at least 91% at least 92%, at least 93%, at least 94%, at least 95%, at least 96%, at least 97%, at least 98%, at least 99% identical to a sequence set forth in SEQ ID NO: 10;
(iv) a VL comprising a sequence at least about 80%, at least 85%, at least 90%, at least 91% at least 92%, at least 93%, at least 94%, at least 95%, at least 96%, at least 97%, at least 98%, at least 99% identical to a sequence set forth in SEQ ID NO: 84;
(v) a VH comprising a CDR1 comprising a sequence set forth in SEQ ID NO: 19, a CDR2 comprising a sequence set forth between in SEQ ID NO: 20, and a CDR3 comprising a sequence set forth in SEQ ID NO: 21;
(vi) a VH comprising a sequence set forth in SEQ ID NO: 83;
(vii) a VL comprising a CDR1 comprising a sequence set SEQ ID NO: 22, a CDR2 comprising a sequence set forth in SEQ ID NO: 23 and a CDR3 comprising a sequence set forth in SEQ ID NO: 10;
(viii) a VL comprising a sequence set forth in SEQ ID NO: 84;
(ix) a VH comprising a CDR1 comprising a sequence set forth in SEQ ID NO: 19, a CDR2 comprising a sequence set forth between in SEQ ID NO: 20 and a CDR3 comprising a sequence set forth in SEQ ID NO: 21; and a VL comprising a CDR1 comprising a sequence set SEQ ID NO: 22, a CDR2 comprising a sequence set forth in SEQ ID NO: 23 and a CDR3 comprising a sequence set forth in SEQ ID NO: 10;
(x) a VH comprising a sequence set forth in SEQ ID NO: 83 and a VL comprising a sequence set forth in SEQ ID NO: 84.

In any aspect of the invention, the antigen binding domain further comprises at least one of:
(i) a VH comprising a framework region (FR) 1 comprising a sequence at least about 80%, at least 85%, at least 90%, at least 91% at least 92%, at least 93%, at least 94%, at least 95%, at least 96%, at least 97%, at least 98%, at least 99% identical to a sequence set forth in SEQ ID NO: 24, a FR2 comprising a sequence at least about 80%, at least 85%, at least 90%, at least 91% at least 92%, at least 93%, at least 94%, at least 95%, at least 96%, at least 97%, at least 98%, at least 99% identical to a sequence set in SEQ ID NO: 25, a FR3 comprising a sequence at least about 80%, at least 85%, at least 90%, at least 91% at least 92%, at least 93%, at least 94%, at least 95%, at least 96%, at least 97%, at least 98%, at least 99% identical to a sequence set forth in SEQ ID NO: 26, and a FR4 comprising a sequence at least about 80%, at least 85%, at least 90%, at least 91% at least 92%, at least 93%, at least 94%, at least 95%, at least 96%, at least 97%, at least 98%, at least 99% identical to a sequence set forth in SEQ ID NO: 14;
(ii) a VL comprising a FR1 comprising a sequence at least about 80%, at least 85%, at least 90%, at least 91% at least 92%, at least 93%, at least 94%, at least 95%, at least 96%, at least 97%, at least 98%, at least 99% identical to a sequence set forth in SEQ ID NO: 27, a FR2 comprising a sequence at least about 80%, at least 85%, at least 90%, at least 91% at least 92%, at least 93%, at least 94%, at least 95%, at least 96%, at least 97%, at least 98%, at least 99% identical to a sequence set forth in SEQ ID NO: 28, a FR3 comprising a sequence at least about 80%, at least 85%, at least 90%, at least 91% at least 92%, at least 93%, at least 94%, at least 95%, at least 96%, at least 97%, at least 98%, at least 99% identical to a sequence set forth in SEQ ID NO: 29, and a FR4 comprising a sequence at least about 80%, at least 85%, at least 90%, at least 91% at least 92%, at least 93%, at least 94%, at least 95%, at least 96%, at least 97%, at least 98%, at least 99% identical to a sequence set forth in SEQ ID NO: 18;
(iii) a VH comprising a FR1 comprising a sequence set forth in SEQ ID NO: 24, a FR2 comprising a sequence set forth between in SEQ ID NO: 25, a FR3 comprising a sequence set forth in SEQ ID NO: 26, and a FR4 comprising a sequence set forth in SEQ ID NO: 14;
(iv) a VL comprising a FR1 comprising a sequence set forth in SEQ ID NO: 27, a FR2 comprising a sequence set forth between in SEQ ID NO: 28, a FR3 comprising a sequence set forth in SEQ ID NO: 29, and a FR4 comprising a sequence set forth in SEQ ID NO: 18; or
(v) a VH comprising a FR1 comprising a sequence set forth in SEQ ID NO: 24, a FR2 comprising a sequence set forth between in SEQ ID NO: 25, a FR3 comprising a sequence set forth in SEQ ID NO: 26, and a FR4 comprising a sequence set forth in SEQ ID NO: 14; and a VL comprising a FR1 comprising a sequence set forth in SEQ ID NO: 27, a FR2 comprising a sequence set forth between in SEQ ID NO: 28, a FR3 comprising a sequence set forth in SEQ ID NO: 29, and a FR4 comprising a sequence set forth in SEQ ID NO: 18.

In any aspect of the invention, the antigen binding domain further comprises at least one of:
(i) a VH comprising a framework region (FR) 1 comprising a sequence at least about 80%, at least 85%, at least 90%, at least 91% at least 92%, at least 93%, at least 94%, at least 95%, at least 96%, at least 97%, at least 98%, at least 99% identical to a sequence set forth in SEQ ID NO: 68, a FR2 comprising a sequence at least about 80%, at least 85%, at least 90%, at least 91% at least 92%, at least 93%, at least 94%, at least 95%, at least 96%, at least 97%, at least 98%, at least 99% identical to a sequence set in SEQ ID NO: 69, a FR3 comprising a sequence at least about 80%, at least 85%, at least 90%, at least 91% at least 92%, at least 93%, at least 94%, at least 95%, at least 96%, at least 97%, at least 98%, at least 99% identical to a sequence set forth in SEQ ID NO: 70, and a FR4 comprising a sequence at least about 80%, at least 85%, at least 90%, at least 91% at least 92%, at least 93%, at least 94%, at least 95%, at least 96%, at least 97%, at least 98%, at least 99% identical to a sequence set forth in SEQ ID NO: 63;
(ii) a VL comprising a FR1 comprising a sequence at least about 80%, at least 85%, at least 90%, at least 91% at least 92%, at least 93%, at least 94%, at least 95%, at least 96%, at least 97%, at least 98%, at least 99% identical to a sequence set forth in SEQ ID NO: 71, a FR2 comprising a sequence at least about 80%, at least 85%, at least 90%, at least 91% at least 92%, at least 93%, at least 94%, at least 95%, at least 96%, at least 97%, at least 98%, at least 99% identical to a sequence set forth in SEQ ID NO: 72, a FR3 comprising a sequence at least about 80%, at least 85%, at least 90%, at least 91% at least 92%, at least 93%, at least 94%, at least 95%, at least 96%, at least 97%, at least 98%, at least 99% identical to a sequence set forth in SEQ ID NO: 73, and a FR4 comprising a sequence at least about 80%, at least 85%, at least 90%, at least 91% at least 92%, at least 93%, at least 94%, at least 95%, at least 96%, at least 97%, at least 98%, at least 99% identical to a sequence set forth in SEQ ID NO: 67;
(iii) a VH comprising a FR1 comprising a sequence set forth in SEQ ID NO: 68, a FR2 comprising a sequence set forth between in SEQ ID NO: 69, a FR3 comprising a sequence set forth in SEQ ID NO: 70, and a FR4 comprising a sequence set forth in SEQ ID NO: 63;
(iv) a VL comprising a FR1 comprising a sequence set forth in SEQ ID NO: 71, a FR2 comprising a sequence set forth between in SEQ ID NO: 72, a FR3 comprising a sequence set forth in SEQ ID NO: 73, and a FR4 comprising a sequence set forth in SEQ ID NO: 67; or
(v) a VH comprising a FR1 comprising a sequence set forth in SEQ ID NO: 68, a FR2 comprising a sequence set forth between in SEQ ID NO: 69, a FR3 comprising a sequence set forth in SEQ ID NO: 70, and a FR4 comprising a sequence set forth in SEQ ID NO: 63; and a VL comprising a FR1 comprising a sequence set forth in SEQ ID NO: 71, a FR2 comprising a sequence set forth between in SEQ ID NO: 72, a FR3 comprising a sequence set forth in SEQ ID NO: 73, and a FR4 comprising a sequence set forth in SEQ ID NO: 67.

As described herein, the antigen binding protein may be in the form of:
(i) a single chain Fv fragment (scFv);
(ii) a dimeric scFv (di-scFv); or
(iii) one of (i) or (ii) linked to a constant region of an antibody, Fc or a heavy chain constant domain (CH) 2 and/or CH3.

Further, as described herein, the antigen binding protein may be in the form of:
(i) a diabody;
(ii) a triabody;
(iii) a tetrabody;
(iv) a Fab;
(v) a F(ab′)2;
(vi) a Fv;
(vii) a bispecific antibody or other form of multispecific antibody; or
(viii) one of (i) to (vii) linked to a constant region of an antibody, Fc or a heavy chain constant domain (CH) 2 and/or CH3.

The foregoing antigen binding proteins can also be referred to as antigen binding domains of antibodies.

In certain embodiments, the complementarity determining region sequences (CDRs) of an antigen binding protein of the invention may be defined according to the IMGT numbering system, Kabat or Chothia systems.

In any aspect or embodiment, an antigen binding protein may comprise human constant regions.

Reference herein to a protein or antibody that “binds to” CCR8 provides literal support for a protein or antibody that “binds specifically to” or “specifically binds to” CCR8.

Preferably, an antigen binding protein as described herein is an antibody or antigen binding fragment thereof. Typically, the antigen binding protein is an antibody, for example, a monoclonal antibody. The antigen binding protein may be in the form of a recombinant or modified antibody (e.g., chimeric antibody, humanised antibody, human antibody, CDR-grafted antibody, primatised antibody, de-immunised antibody, synhumanised antibody, half-antibody, bispecific antibody, trispecific antibody or multispecific antibody). The antibody may further comprise a chemical modification, such as conjugation to an active agent or radiolabel, or an agent for improving solubility or other modification described herein.

In any aspect of the invention, the antigen binding protein further comprises one or more human constant regions. In one embodiment, the antigen binding protein comprises the amino acid sequences shown in SEQ ID NO: 58 and/or 59.

In another aspect, the present invention also provides an anti-CCR8 antibody or antigen binding fragment thereof, comprising a light chain variable region and a heavy chain variable region,
wherein said heavy chain variable region comprises:
   a CDR H1 as set forth in SEQ ID NO: 5, a CDR H2 as set forth in SEQ ID NO: 6, and a CDR H3 as set forth in SEQ ID NO: 7; and
wherein said light chain variable region comprises:
   a CDR L1 as set forth in SEQ ID NO: 8, a CDR L2 as set forth in SEQ ID NO: 9 and a CDR L3 as set forth in SEQ ID NO: 10.

In any embodiment, the anti-CCR8 antibody or antigen binding fragment thereof comprises a light chain variable region that comprises a FR L1 as set forth in SEQ ID NO: 15, an FR L2 as set forth in SEQ ID NO: 16, a FR L3 as set forth in SEQ ID NO: 17 and a FR L4 as set forth in SEQ ID NO: 18.

In any embodiment of the invention, the anti-CCR8 antibody or antigen binding fragment thereof comprises a heavy chain variable region that comprises a FR H1 as set forth in SEQ ID NO: 11, FR H2 as set forth in SEQ ID NO: 12, a FR H3 as set forth in SEQ ID NO: 13 and a FR H4 as set forth in SEQ ID NO: 14.

In any embodiment, the anti-CCR8 antibody or antigen binding fragment thereof comprises a light chain variable region that comprises a FR L1 as set forth in SEQ ID NO: 64, an FR L2 as set forth in SEQ ID NO: 65, a FR L3 as set forth in SEQ ID NO: 66 and a FR L4 as set forth in SEQ ID NO: 67.

In any embodiment of the invention, the anti-CCR8 antibody or antigen binding fragment thereof comprises a heavy chain variable region that comprises a FR H1 as set forth in SEQ ID NO: 60, FR H2 as set forth in SEQ ID NO: 61, a FR H3 as set forth in SEQ ID NO: 62 and a FR H4 as set forth in SEQ ID NO: 63.

In another aspect, the present invention also provides an anti-CCR8 antibody or antigen binding fragment thereof, comprising a light chain variable region and a heavy chain variable region,
wherein said heavy chain variable region comprises:
   a CDR H1 as set forth in SEQ ID NO: 19, a CDR H2 as set forth in SEQ ID NO: 20, and a CDR H3 as set forth in SEQ ID NO: 21; and
wherein said light chain variable region comprises:
   a CDR L1 as set forth in SEQ ID NO: 22, a CDR L2 as set forth in SEQ ID NO: 23 and a CDR L3 as set forth in SEQ ID NO: 10.

In any embodiment, the anti-CCR8 antibody or antigen binding fragment thereof comprises a light chain variable region that comprises a FR L1 as set forth in SEQ ID NO: 27, an FR L2 as set forth in SEQ ID NO: 28, a FR L3 as set forth in SEQ ID NO: 29 and a FR L4 as set forth in SEQ ID NO: 18.

In any embodiment of the invention, the anti-CCR8 antibody or antigen binding fragment thereof comprises a heavy chain variable region that comprises a FR H1 as set forth in SEQ ID NO: 24, FR H2 as set forth in SEQ ID NO: 25, a FR H3 as set forth in SEQ ID NO: 26 and a FR H4 as set forth in SEQ ID NO: 14.

In any embodiment, the anti-CCR8 antibody or antigen binding fragment thereof comprises a light chain variable region that comprises a FR L1 as set forth in SEQ ID NO: 71, an FR L2 as set forth in SEQ ID NO: 72, a FR L3 as set forth in SEQ ID NO: 73 and a FR L4 as set forth in SEQ ID NO: 67.

In any embodiment of the invention, the anti-CCR8 antibody or antigen binding fragment thereof comprises a heavy chain variable region that comprises a FR H1 as set forth in SEQ ID NO: 68, FR H2 as set forth in SEQ ID NO: 69, a FR H3 as set forth in SEQ ID NO: 70 and a FR H4 as set forth in SEQ ID NO: 63.

In any embodiment, the anti-CCR8 receptor antibody or antigen binding fragment thereof, comprises a light chain variable region that comprises the sequence of SEQ ID NO: 2.

In any embodiment, the anti-CCR8 antibody or antigen binding fragment thereof, comprises a heavy chain variable region that comprises the sequence of SEQ ID NO: 1.

In any embodiment, the anti-CCR8 receptor antibody or antigen binding fragment thereof, comprises a light chain variable region that comprises the sequence of SEQ ID NO: 84.

In any embodiment, the anti-CCR8 antibody or antigen binding fragment thereof, comprises a heavy chain variable region that comprises the sequence of SEQ ID NO: 83.

In any embodiment, the anti-CCR8 receptor antibody or antigen binding fragment thereof, comprises a light chain variable region that comprises the sequence of SEQ ID NO: 2 and a heavy chain variable region that comprises the sequence of SEQ ID NO: 1.

In any embodiment, the anti-CCR8 receptor antibody or antigen binding fragment thereof, comprises a light chain variable region that comprises the sequence of SEQ ID NO: 84 and a heavy chain variable region that comprises the sequence of SEQ ID NO: 83.

In any aspect or embodiment, the antigen binding protein or anti-CCR8 receptor antibody or antigen binding fragment thereof may further comprises at most 15, preferably, 0, 1, 2, 3, 4, 5, 6, 7, 8, 9, or 10, and preferably having 0, 1, 2, 3, 4 or 5 amino acid insertions, deletions, substitutions or a combination thereof with respect to the amino acid sequence of the indicated SEQ ID NO. Preferably, the amino acid insertions, deletions, substitutions or a combination thereof are not in the CDRs.

In any aspect of the invention and in any antigen binding protein described herein, there further includes an Fc region that is engineered to have reduced or increased capacity to induce antibody-dependent cell-mediated cytotoxicity (ADCC). Preferably, the reduced or increased capacity to induce ADCC is conferred by mutation, deletion or modification of amino acids in the Fc region which interact with an Fc receptor.

In another aspect, the invention provides an anti-CCR8 antigen binding protein, immunoglobulin variable domain, antibody or antigen binding fragment thereof, scFv, Fab, Fab′, F(ab′)2, Fv fragment, diabody, triabody, linear antibody, single-chain antibody molecule, or multispecific antibody comprising an antigen binding protein having a sequence as described herein, or including a CDR and/or FR sequence as described herein.

In any aspect, the antigen binding protein is an antibody of IgG2b isotype.

An antigen binding protein as described herein may comprise a human constant region, e.g., an IgG constant region, such as an IgG₁, IgG₂, IgG₃ or IgG₄ constant region or mixtures thereof. In the case of an antibody or protein comprising a V_{H} and a V_{L}, the V_{H} can be linked to a heavy chain constant region and the V_{L} can be linked to a light chain constant region.

In one example, an antigen binding protein as described herein comprises a constant region of an IgG₄ antibody or a stabilised constant region of an IgG₄ antibody. In one example, the protein or antibody comprises an IgG₄ constant region with a proline at position 241 (according to the numbering system of Kabat (Kabat et al., Sequences of Proteins of Immunological Interest Washington DC United States Department of Health and Human Services, 1987 and/or 1991)).

In one example, an antigen binding protein comprises a V_{H} disclosed herein linked or fused to an IgG₄ constant region or stabilised IgG₄ constant region (e.g., as discussed above) and the V_{L} is linked to or fused to a kappa light chain constant region.

In any aspect of the present invention, the antibody is a naked antibody. Specifically, the antibody is in a non-conjugated form and is not adapted to form a conjugate.

In any aspect of the present invention, the antibody exhibits antibody-dependent cell-mediated cytotoxicity (ADCC). Preferably the antibody exhibits ADCC to the same or similar level as 19D7 (anti-CCR8 Shionogi antibody). ADCC may be measured by any method known in the art or as described herein, including Example 11.

In any aspect of the present invention, the antigen binding protein depletes or reduces tumour infiltrating T regulatory (Tregs) cells in a tumour; optionally wherein the antigen binding protein does not deplete or reduce Tregs in the spleen.

In any aspect of the present invention, the antigen binding protein increases tumour-specific CD8+ T cells and/or, reduces CD4+ T cells or tumour-specific CD4+ T cells.

In another aspect, the invention also provides a conjugate in the form of an antigen binding protein, immunoglobulin variable domain, antibody or antigen binding fragment thereof, scFv, Fab, Fab′, F(ab′)2, Fv fragment, diabody, triabody, linear antibody, single-chain antibody molecule, or multispecific antibody or fusion protein as described herein conjugated to a label or a cytotoxic agent.

In aspects of the invention directed to multiple polypeptide chains that form an antigen binding protein, an expression construct comprises a nucleic acid encoding a polypeptide comprising, e.g., a VH operably linked to a promoter and a nucleic acid encoding a polypeptide comprising, e.g., a VL operably linked to a promoter.

In another example, the expression construct is a bicistronic expression construct, e.g., comprising the following operably linked components in 5′ to 3′ order:
(i) a promoter
(ii) a nucleic acid encoding a first polypeptide;
(iii) an internal ribosome entry site; and
(iv) a nucleic acid encoding a second polypeptide,
wherein the first polypeptide comprises a VH and the second polypeptide comprises a VL, or vice versa.

In another aspect, the present invention also contemplates separate expression constructs one of which encodes a first polypeptide comprising a VH and another of which encodes a second polypeptide comprising a VL. For example, the present invention also provides a composition comprising:
(i) a first expression construct comprising a nucleic acid encoding a polypeptide comprising a VH operably linked to a promoter; and
(ii) a second expression construct comprising a nucleic acid encoding a polypeptide comprising a VL operably linked to a promoter.

In another aspect, the invention provides a cell comprising a vector or nucleic acid described herein. Preferably, the cell is isolated, substantially purified or recombinant. In one example, the cell comprises the expression construct of the invention or:
(i) a first expression construct comprising a nucleic acid encoding a polypeptide comprising a VH operably linked to a promoter; and
(ii) a second expression construct comprising a nucleic acid encoding a polypeptide comprising a VL operably linked to a promoter,
wherein the first and second polypeptides associate to form an antigen binding protein of the present invention.

Examples of cells of the present invention include bacterial cells, yeast cells, insect cells or mammalian cells.

In another aspect, the invention provides a nucleic acid encoding an antigen binding protein, immunoglobulin variable domain, antibody or antigen binding fragment thereof, scFv, Fab, Fab′, F(ab′)2, Fv fragment, diabody, triabody, linear antibody, single-chain antibody molecule, or multispecific antibody, fusion protein or conjugate as described herein.

In another aspect, the invention provides a vector comprising a nucleic acid described herein.

In another aspect, the invention provides a cell comprising a vector or nucleic acid described herein.

In any embodiment, the nucleic acid may comprise a nucleotide sequence as set forth in any of SEQ ID NOs: 3, 4 or 30 to 54, preferably SEQ ID NO: 3 and/or 4, or sequences at least about 80%, at least 85%, at least 90%, at least 91% at least 92%, at least 93%, at least 94%, at least 95%, at least 96%, at least 97%, at least 98%, at least 99% identical thereto.

In another aspect, the invention provides a pharmaceutical composition comprising an antigen binding protein, or including a CDR and/or FR sequence as described herein, or an immunoglobulin variable domain, antibody or antigen binding fragment thereof, scFv, Fab, Fab′, F(ab′)2, Fv fragment, diabody, triabody, linear antibody, single-chain antibody molecule, or multispecific antibody, fusion protein, or conjugate as described herein and a pharmaceutically acceptable carrier, diluent or excipient.

In another aspect, the invention provides a kit or article of manufacture comprising an antigen binding protein, or including a CDR and/or FR sequence as described herein or an immunoglobulin variable domain, antibody or antigen binding fragment thereof, scFv, Fab, Fab′, F(ab′)2, Fv fragment, diabody, triabody, linear antibody, single-chain antibody molecule, or multispecific antibody, fusion protein or conjugate as described herein.

In another aspect, the invention provides use of a sequence according to one or more of CDR1, CDR2, CDR3, FR1, FR2, FR3 and FR4 as described herein to produce an antigen binding protein for binding to CCR8.

In another aspect, the invention provides a method for producing an antigen binding protein, antibody or antigen binding fragment for binding to CCR8 as described herein comprising expressing a nucleic acid as described herein in a cell or animal as described herein.

The functional characteristics of an antigen binding protein of the invention will be taken to apply mutatis mutandis to an antibody or antigen binding fragment of the invention.

An antigen binding protein as described herein may be purified, substantially purified, isolated and/or recombinant.

An antigen binding protein of the invention may be part of a supernatant taken from media in which a hybridoma expressing an antigen binding protein of the invention has been grown.

In another aspect, the invention provides a method for the prevention or treatment a condition or disease associated with expression of CCR8 in an individual comprising the step of providing an antigen binding protein, immunoglobulin variable domain, antibody or antigen binding fragment thereof, scFv, Fab, Fab′, F(ab′)2, Fv fragment, diabody, triabody, linear antibody, single-chain antibody molecule, or multispecific antibody, fusion protein, conjugate or pharmaceutical composition as described herein to an individual requiring treatment for said condition or disease. The disease or condition associated with expression of CCR8 is preferably a cancer.

In another aspect, the present invention also provides for a method of treating or preventing a cancer in a subject, the method comprising administering an antigen binding protein, antibody or antigen binding fragment or a pharmaceutical composition of the invention to the subject, thereby treating or preventing a cancer in the subject. As used herein, methods of treating cancer include methods of inhibiting, preventing or minimising spread or progression of a cancer, including inhibiting or preventing metastasis of cancer.

In another aspect, the present invention also provides for a method of reducing or depleting T regulatory cells in a tumour, the method comprising administering an antigen binding protein, antibody or antigen binding fragment or a pharmaceutical composition of the invention to the subject, thereby reducing or depleting T regulatory cells in a tumour.

In another aspect, the present invention also provides for a method of increasing tumor-specific CD8+ T-cells and/or reducing CD4+ T-cells in a tumour, the method comprising administering an antigen binding protein, antibody or antigen binding fragment or a pharmaceutical composition of the invention to the subject, thereby increasing tumor-specific CD8+ T-cells and/or reducing CD4+ T-cells in a tumour.

In another aspect, the present invention also provides for the use of an antigen binding protein, antibody or antigen binding fragment or a pharmaceutical composition of the invention, in the manufacture of a medicament for the treatment or prevention of cancer in a subject.

In another aspect, the present invention also provides the use of an antigen binding protein, antibody or antigen binding fragment or a pharmaceutical composition of the invention, in the manufacture of a medicament for:
reducing or depleting T regulatory cells in a tumour; or

$$increasing tumor - specific CD 8 + T - cells and / or reducing CD 4 + T - cells in a tumour .$$

In another aspect, the invention provides for an antigen binding protein, antibody or antigen binding fragment or a pharmaceutical composition of the invention, for use in the treatment or prevention of cancer in a subject.

In another aspect, the invention provides for an antigen binding protein, antibody or antigen binding fragment or a pharmaceutical composition of the invention, for use in:
reducing or depleting T regulatory cells in a tumour; or

$$increasing tumor - specific CD 8 + T - cells and / or reducing CD 4 + T - cells in a tumour .$$

As used herein, except where the context requires otherwise, the term “comprise” and variations of the term, such as “comprising”, “comprises” and “comprised”, are not intended to exclude further additives, components, integers or steps.

Further aspects of the present invention and further embodiments of the aspects described in the preceding paragraphs will become apparent from the following description, given by way of example and with reference to the accompanying drawings.

### BRIEF DESCRIPTION OF THE DRAWINGS

FIG. 1. Reactivity of 2H8 antibody toward both human and mouse in transfected CHO-Flpn cell lines. 2H8 reactivity with mouse CCR8 (blue) or human CCR8 (orange) CHO-Flpn cells and untransfected CHO Flpn (red) compared to (A) anti-hCCR8 #1 antibody or (B) anti-hCCR8 (Clone #L263G8 Biolegend).

FIG. 2. Reactivity of 2H8 antibody and other anti-hCCR8 antibodies towards Cyno CCR8. HEK293T cells were transfected with plasmid coding for cynomolgus monkey CCR8 and stained with various anti-hCCR8 antibodies (blue) or isotype control (red).

FIG. 3. Reactivity of 2H8 antibody towards other CCR chemokine receptors in transfected L1.2 cells. 2H8-B4 doesn't cross react with untransfected cells (purple); human CCR3 (pink); human CCR6 (dark green); human CCR7 (lime) or human CCR10 (blue)—in order of top to bottom for non-binding histograms. 2H8-B4 binds to both human CCR8 (orange) and mouse CCR8 (red) transfected L1.2 cells—in order of top to bottom for binding histograms.

FIG. 4. 2H8 binds to human tumour infiltrating Tregs (TiTregs). Fresh colorectal patient biopsy was used to isolated single cell suspension. Cells were stained with anti-human CD3; anti-human CD4; anti-human FoxP3, and either 2H8-B4 or isotype control. Histograms show that 2H8-B4 (red) binds to tumor infiltrating Tregs (CD3+; CD4+; FoxP3+ gate) while isotype control doesn't (grey).

FIG. 5. Binding of hCCR8 antibodies to hCCR8-L1.2 transfected cells. Chimeric 2H8-B4 antibody was compared to chimeric 19D7 (Shionogi antibody) and chimeric 4A19 (BMS antibody) by flow cytometry on hCCR8-L1.2 transfected cells using increasing concentrations of purified mAbs.

FIG. 6. Binding of humanized 2H8 to hCCR8-L1.2 transfected cells. Humanized 2H8-B4 was compared to chimeric 2H8 B4 (parental) and 19D7 (Shionogi antibody) by flow cytometry on hCCR8-L1.2 transfected cells using increasing concentrations of purified mAbs.

FIG. 7. Fresh colorectal patient biopsy was used to isolated single cell suspension. Cells were stained with anti-human CD3; anti-human CD4; anti-human FoxP3, and either 2H8-B4 (right) or isotype control (left). 2H8-B4 (right) binds to tumor infiltrating Tregs (CD3+; CD4+; FoxP3+ gate) while isotype control doesn't (left).

FIG. 8. Chimeric 2H8 doesn't inhibit CCL1 induce chemotaxis. 200,000 hCCR8 L1.2 cells/well were pre-incubated with different concentration of antibodies for 15 min at 37° C. Cells were then transferred onto chemotaxis plate and incubated overnight (4 hour) for chemotaxis towards EC90 (hCCL1—5.84 nM) with the same concentration gradient of antibodies. 100 ul were then collected from the bottom well, spun down and resuspend in 50 ul FACS buffer. 15 ul were then counted on the LSRIIb machine.

FIG. 9. Chimeric 2H8 exhibits ADCC Potential with Efficacy and Potency comparable to 19D7. A dose-response curve of an ADCC assay conducted to analyze the potential to induce cellular cytotoxicity of both chimeric 2H8 and chimeric 19D7 by using L1.2-hCCR8 cells incubated with various antibody concentrations. The highest antibody concentration was 13.33 nM and was diluted four-fold to create a dose-response curve. Antibody concentration was converted into a Log scale and placed on the x-axis. Statistical analysis was done using a sigmoidal standard line of best fit to calculate the IC50 and R2 of each antibody.

FIG. 10. 2H8 inhibits the tumor growth in an MC38 model of colorectal cancer. Plots of individual volume of MC38 tumors implanted sub-cutaneously at 5×10⁵ cells/injection in C57BL/6 mice. Animals were treated with 2H8 mlgG2a (n=9), anti-mPD1 (n=10), and isotype control (n=6). Tumor volume (mm³) was measured daily with a digital calliper after the first injection of antibodies and compared between groups. Treatment was conducted 2 times/week with a dose of 5 mg/kg for a maximum duration of 3 weeks.

FIG. 11. 2H8 caused Tregs depletion in the tumor but not in the spleen. At the endpoint, tumor and spleen tissues were processed, stained, and analyzed on flow cytometry. The percentage of CD4⁺FOXP3⁺CD25⁺ cells in TCR-β⁺ cells (parent population) was collected and compared between the three groups. All graphs present mean and individual values with histogram, scatter plot, and error bars representing SEM. (A) Representative flow cytometry scatter plot of the three groups with mean±SEM of percentage of CD4⁺FOXP3⁺CD25⁺ cells displayed. (B) T-regs percentage in the tumor of C57BL/6 wild type mice. (C) T-regs percentage in the spleen of C57BL/6 wild type mice. 2H8 group; n=9, Anti-PD-1 group; n=10, control group; n=6. Normality test using the Shapiro-Wilk method (b) Kruskal-Wallis with Dunn's multiple comparisons (c) Ordinary one-way ANOVA with Tukey's multiple comparisons. *p<0.05, **p<0.005.

FIG. 12. 2H8 treatment increases tumor-specific CD8+ T-cells and reduces CD4+ T-cells. At the endpoint, tumor and spleen tissues of MC38 tumors in C57BL/6 mice were processed, stained, and analyzed with flow cytometry. The percentage of CD4+ and CD8+ cells in TCR-β+ cells (parent population) was collected and compared between the three groups. All graphs present mean and individual values with histogram, scatter plot, and error bars representing SEM. (A) Representative flow cytometry scatter plot of the tumors of the three different groups with mean±SEM of CD8+ T-cells displayed. (B) Percentage of CD8+ T-cells in the tumor. (C) CD8/T-regs ratio normalized to tumor weight. (D) Percentage of CD4+ T-cells in the tumor. (E) Representative flow cytometry scatter plot of CD62L and CD44 marker on the tumors of the three different groups. (F) Percentage of CD62LloCD44hi 008+ T-cells in the tumor. (G) Percentage of CD62LhiCD44hi CD8+ T-cells in the tumor. (H) Percentage of CD62LloCD44lo 008+ T-cells in the tumor. (I) Percentage of CD62LhiCD44lo CD8+ T-cells in the tumor. 2H8 group; n=9, Anti-PD-1 group; n=10, control group; n=6. Normality test using the Shapiro-Wilk method (b, d, e, g, h) Ordinary one-way ANOVA with Tukey's multiple comparisons. (c) Kruskal-Wallis with Dunn's multiple comparisons *p<0.05, **p<0.005, ***p=0.0001, ****p<0.0001

### DESCRIPTION OF SEQUENCES

TABLE 1
Sequences of the invention

| SEQ ID | | |
|---|---|---|
| NO | Description | Sequence |
| 1 | 2H8_anti-m/hCCR8_VH | EVQLQQSGPELVKPGASVKMSCKASGYTFTDYNMHWVKQSHGKS |
| | | LEWIGYVNPNNGGTSYNQKFKGKATLTVNKSSSTAYMELRSLTS |
| | | EDSAVYYCARWVLRRNFAYWGQGTLVTVSA |
| 2 | 2H8_anti-m/hCCR8_VK | DIVMTQSHKFMSTSVGDRVSITCKASQDVNTAVAWYQQKPGQSPK |
| | | LLIYWASTRHTGVPDRFTGSGSGTDYTLTISSVQAEDLALYYCQQ |
| | | HYRNPYTFGGGTKLEIK |
| 3 | 2H8-B4_VH_DNA | GAGGTCCAGCTGCAACAGTCTGGACCTGAGCTGGTGAAGCCTG |
| | | GGGCTTCAGTGAAGATGTCCTGCAAGGCTTCTGGATACACATTC |
| | | ACTGACTACAACATGCACTGGGTGAAGCAAAGCCATGGAAAGAG |
| | | CCTTGAGTGGATTGGATATGTTAACCCTAACAATGGTGGTACTAG |
| | | CTACAACCAGAAGTTCAAGGGCAAGGCCACATTGACTGTAAACA |
| | | AGTCCTCCAGCACAGCCTACATGGAGCTCCGCAGCCTGACATCG |
| | | GAGGATTCTGCAGTCTATTACTGTGCAAGATGGGTATTACGACG |
| | | GAACTTTGCTTACTGGGGCCAAGGGACTCTGGTCACTGTCTCTG |
| | | CA |
| 4 | 2H8-B4_VK_DNA | GACATTGTGATGACCCAGTCTCACAAATTCATGTCCACATCAGTA |
| | | GGAGACAGGGTCAGCATCACCTGCAAGGCCAGTCAGGATGTGA |
| | | ATACTGCTGTAGCCTGGTATCAACAAAAACCAGGGCAATCTCCTA |
| | | AACTACTGATTTACTGGGCATCCACCCGGCACACTGGAGTCCCT |
| | | GATCGCTTCACAGGCAGTGGATCTGGGACAGATTATACTCTCAC |
| | | CATCAGCAGTGTGCAGGCTGAAGACCTGGCACTTTATTACTGTCA |
| | | GCAACATTATAGAAATCCGTACACGTTCGGAGGGGGGACCAAGC |
| | | TGGAAATAAAA |
| 5 | 2H8-B4 CDRH1 (IMGT) | GYTFTDYN |
| 6 | 2H8-B4 CDRH2 (IMGT) | VNPNNGGT |
| 7 | 2H8-B4 CDRH3 (IMGT) | ARWVLRRNFAY |
| 8 | 2H8-B4 CDRL1 (IMGT) | QDVNTA |
| 9 | 2H8-B4 CDRL2 (IMGT) | WAS |
| 10 | 2H8-B4 CDRL3 (IMGT | QQHYRNPYT |
| | and Kabat) | |
| 11 | 2H8-B4 FRH1 (IMGT) | EVQLQQSGPELVKPGASVKMSCKAS |
| 12 | 2H8-B4 FRH2 (IMGT) | MHWVKQSHGKSLEWIGY |
| 13 | 2H8-B4 FRH3 (IMGT) | SYNQKFKGKATLTVNKSSSTAYMELRSLTSEDSAVYYC |
| 14 | 2H8-B4 FRH4 (IMGT | WGQGTLVTVSA |
| | and Kabat) | |
| 15 | 2H8-B4 FRL1 (IMGT) | DIVMTQSHKFMSTSVGDRVSITCKAS |
| 16 | 2H8-B4 FRL2 (IMGT) | VAWYQQKPGQSPKLLIY |
| 17 | 2H8-B4 FRL3 (IMGT) | TRHTGVPDRFTGSGSGTDYTLTISSVQAEDLALYYC |
| 18 | 2H8-B4 FRL4 (IMGT | FGGGTKLEIK |
| | and Kabat) | |
| 19 | 2H8-B4 CDRH1 (Kabat) | DYNMH |
| 20 | 2H8-B4 CDRH2 (Kabat) | YVNPNNGGTSYNQKFKG |
| 21 | 2H8-B4 CDRH3 (Kabat) | WLRRNFAY |
| 22 | 2H8-B4 CDRL1 (Kabat) | KASQDVNTAVA |
| 23 | 2H8-B4 CDRL2 (Kabat) | WASTRHT |
| 24 | 2H8-B4 FRH1 (Kabat) | EVQLQQSGPELVKPGASVKMSCKASGYTFT |
| 25 | 2H8-B4 FRH2 (Kabat) | WVKQSHGKSLEWIG |
| 26 | 2H8-B4 FRH3 (Kabat) | KATLTVNKSSSTAYMELRSLTSEDSAVYYCAR |
| 27 | 2H8-B4 FRL1 (Kabat) | DIVMTQSHKFMSTSVGDRVSITC |
| 28 | 2H8-B4 FRL2 (Kabat) | WYQQKPGQSPKLLIY |
| 29 | 2H8-B4 FRL3 (Kabat) | GVPDRFTGSGSGTDYTLTISSVQAEDLALYYC |
| 30 | 2H8-B4 CDRH1_DNA | GGATACACATTCACTGACTACAAC |
| | (IMGT) | |
| 31 | 2H8-B4 CDRH2_DNA | GTTAACCCTAACAATGGTGGTACT |
| | (IMGT) | |
| 32 | 2H8-B4 CDRH3_DNA | GCAAGATGGGTATTACGACGGAACTTTGCTTAC |
| | (IMGT) | |
| 33 | 2H8-B4 CDRL1_DNA | CAGGATGTGAATACTGCT |
| | (IMGT) | |
| 34 | 2H8-B4 CDRL2_DNA | TGGGCATCC |
| | (IMGT) | |
| 35 | 2H8-B4 CDRL3_DNA | CAGCAACATTATAGAAATCCGTACACG |
| | (IMGT and Kabat) | |
| 36 | 2H8-B4 FRH1_DNA | GAGGTCCAGCTGCAACAGTCTGGACCTGAGCTGGTGAAGCCTG |
| | (IMGT) | GGGCTTCAGTGAAGATGTCCTGCAAGGCTTCT |
| 37 | 2H8-B4 FRH2_DNA | ATGCACTGGGTGAAGCAAAGCCATGGAAAGAGCCTTGAGTGGAT |
| | (IMGT) | TGGATAT |
| 38 | 2H8-B4 FRH3_DNA | AGCTACAACCAGAAGTTCAAGGGCAAGGCCACATTGACTGTAAA |
| | (IMGT) | CAAGTCCTCCAGCACAGCCTACATGGAGCTCCGCAGCCTGACAT |
| | | CGGAGGATTCTGCAGTCTATTACTGT |
| 39 | 2H8-B4 FRH4_DNA | TGGGGCCAAGGGACTCTGGTCACTGTCTCTGCA |
| | (IMGT and Kabat) | |
| 40 | 2H8-B4 FRL1_DNA | GACATTGTGATGACCCAGTCTCACAAATTCATGTCCACATCAGTA |
| | (IMGT) | GGAGACAGGGTCAGCATCACCTGCAAGGCCAGT |
| 41 | 2H8-B4 FRL2_DNA | GTAGCCTGGTATCAACAAAAACCAGGGCAATCTCCTAAACTACTG |
| | (IMGT) | ATTTAC |
| 42 | 2H8-B4 FRL3_DNA | ACCCGGCACACTGGAGTCCCTGATCGCTTCACAGGCAGTGGATC |
| | (IMGT) | TGGGACAGATTATACTCTCACCATCAGCAGTGTGCAGGCTGAAG |
| | | ACCTGGCACTTTATTACTGT |
| 43 | 2H8-B4 FRL4_DNA | TTCGGAGGGGGGACCAAGCTGGAAATAAAA |
| | (IMGT and Kabat) | |
| 44 | 2H8-B4 CDRH1_DNA | GACTACAACATGCAC |
| | (Kabat) | |
| 45 | 2H8-B4 CDRH2_DNA | TATGTTAACCCTAACAATGGTGGTACTAGCTACAACCAGAAGTTC |
| | (Kabat) | AAGGGC |
| 46 | 2H8-B4 CDRH3_DNA | TGGGTATTACGACGGAACTTTGCTTAC |
| | (Kabat) | |
| 47 | 2H8-B4 CDRL1_DNA | AAGGCCAGTCAGGATGTGAATACTGCTGTAGCC |
| | (Kabat) | |
| 48 | 2H8-B4 CDRL2_DNA | TGGGCATCCACCCGGCACACT |
| | (Kabat) | |
| 49 | 2H8-B4 FRH1_DNA | GAGGTCCAGCTGCAACAGTCTGGACCTGAGCTGGTGAAGCCTG |
| | (Kabat) | GGGCTTCAGTGAAGATGTCCTGCAAGGCTTCTGGATACACATTC |
| | | ACT |
| 50 | 2H8-B4 FRH2_DNA | TGGGTGAAGCAAAGCCATGGAAAGAGCCTTGAGTGGATTGGA |
| | (Kabat) | |
| 51 | 2H8-B4 FRH3_DNA | AAGGCCACATTGACTGTAAACAAGTCCTCCAGCACAGCCTACAT |
| | (Kabat) | GGAGCTCCGCAGCCTGACATCGGAGGATTCTGCAGTCTATTACT |
| | | GTGCAAGA |
| 52 | 2H8-B4 FRL1_DNA | GACATTGTGATGACCCAGTCTCACAAATTCATGTCCACATCAGTA |
| | (Kabat) | GGAGACAGGGTCAGCATCACCTGC |
| 53 | 2H8-B4 FRL2_DNA | TGGTATCAACAAAAACCAGGGCAATCTCCTAAACTACTGATTTAC |
| | (Kabat) | |
| 54 | 2H8-B4 FRL3_DNA | GGAGTCCCTGATCGCTTCACAGGCAGTGGATCTGGGACAGATTA |
| | (Kabat) | TACTCTCACCATCAGCAGTGTGCAGGCTGAAGACCTGGCACTTT |
| | | ATTACTGT |
| 55 | Human CCR8 | MDYTLDLSVTTVTDYYYPDIFSSPCDAELIQTNGKLLLAVFYCLLFVF |
| | | SLLGNSLVILVLVVCKKLRSITDVYLLNLALSDLLFVFSFPFQTYYLL |
| | | DQWVFGTVMCKVVSGFYYIGFYSSMFFITLMSVDRYLAVVHAVYALK |
| | | VRTIRMGTTLCLAVWLTAIMATIPLLVFYQVASEDGVLQCYSFYNQQT |
| | | LKWKIFTNFKMNILGLLIPFTIFMFCYIKILHQLKRCQNHNKTKAIRL |
| | | VLIVVIASLLFWVPFNVVLFLTSLHSMHILDGCSISQQLTYATHVTEI |
| | | ISFTHCCVNPVIYAFVGEKFKKHLSEIFQKSCSQIFNYLGRQMPRESC |
| | | EKSSSCQQHSSRSSSVDYIL |
| 56 | Mouse CCR8 | MDYTMEPNVTMTDYYPDFFTAPCDAEFLLRGSMLYLAILYCVLFVLGL |
| | | LGNSLVILVLVGCKKLRSITDIYLLNLAASDLLFVLSIPFQTHNLLDQ |
| | | WVFGTAMCKVVSGLYYIGFFSSMFFITLMSVDRYLAIVHAVYAIKVRT |
| | | ASVGTALSLTVWLAAVTATIPLMVFYQVASEDGMLQCFQFYEEQSLR |
| | | WKLFTHFEINALGLLLPFAILLFCYVRILQQLRGCLNHNRTRAIKLVL |
| | | TWVIVSLLFWVPFNVALFLTSLHDLHILDGCATRQRLALAIHVTEVISF |
| | | THCCVNPVIYAFIGEKFKKHLMDVFQKSCSHIFLYLGRQMPVGALER |
| | | QLSSNQRSSHSSTLDDIL |
| 57 | Cynomolgus CCR8 | MDYTLDPSMTTMTDYYYPDSLSSPCDGELIQRNDKLLLAVFYCLLFVF |
| | | SLLGNSLVILVLVVCKKLRNITDIYLLNLALSDLLFVFSFPFQTYYQL |
| | | DQWVFGTVMCKVVSGFYYIGFYSSMFFITLMSVDRYLAVVHAVYAIKV |
| | | RTIRMGTTLSLVVWLTAIMATIPLLVFYQVASEDGVLQCYSFYNQQTL |
| | | KWKIFTNFEMNILGLLIPFTIFMFCYIKILHQLKRCQNHNKTKAIRLV |
| | | LIVVIASLLFWVPFNVVLFLTSLHSMHILDGCSISQQLNYATHVTEII |
| | | SFTHCCVNPVIYAFVGEKFKKHLSEIFQKSCSHIFIYLGRQMPRESCE |
| | | KSSSCQQHSFRSSSIDYIL |
| 58 | Human heavy chain | ASTKGPSVFPLAPSSKSTSGGTAALGCLVKDYFPEPVTVSWNSGAL |
| | constant region (IgG1) | TSGVHTFPAVLQSSGLYSLSSVVTVPSSSLGTQTYICNVNHKPSNTK |
| | | VDKKVEPKSCDKTHTCPPCPAPELLGGPSVFLFPPKPKDTLMISRTP |
| | | EVTCVVVDVSHEDPEVKFNWYVDGVEVHNAKTKPREEQYNSTYRV |
| | | VSVLTVLHQDWLNGKEYKCKVSNKALPAPIEKTISKAKGQPREPQVY |
| | | TLPPSRDELTKNQVSLTCLVKGFYPSDIAVEWESNGQPENNYKTTP |
| | | PVLDSDGSFFLYSKLTVDKSRWQQGNVFSCSVMHEALHNHYTQKS |
| | | LSLSPGK |
| 59 | Human light chain | RTVAAPSVFIFPPSDEQLKSGTASVVCLLNNFYPREAKVQWKVDNA |
| | constant region (Kappa) | LQSGNSQESVTEQDSKDSTYSLSSTLTLSKADYEKHKVYACEVTHQ |
| | | GLSSPVTKSFNRGEC |
| 60 | h2H8-B4 FRH1 (IMGT) | QVQLVQSGAEVKKPGASVKVSCKAS |
| 61 | h2H8-B4 FRH2 (IMGT) | MHWVRQAPGQGLEWIGY |
| 62 | h2H8-B4 FRH3 (IMGT) | SYNQKFKGKATLTVDKSISTAYMELSRLRSDDTAVYYC |
| 63 | h2H8-B4 FRH4 (IMGT | WGQGTLVTVSS |
| | and Kabat) | |
| 64 | h2H8-B4 FRL1 (IMGT) | DIQMTQSPSSLSASVGDRVTITCKAS |
| 65 | h2H8-B4 FRL2 (IMGT) | VAWYQQKPGKAPKLLIY |
| 66 | h2H8-B4 FRL3 (IMGT) | TRHTGVPSRFSGSGSGTDFTLTISSLQPEDFATYYC |
| 67 | h2H8-B4 FRL4 (IMGT | FGQGTKLEIK |
| | and Kabat) | |
| 68 | h2H8-B4 FRH1 (Kabat) | QVQLVQSGAEVKKPGASVKVSCKASGYTFT |
| 69 | h2H8-B4 FRH2 (Kabat) | WVRQAPGQGLEWIG |
| 70 | h2H8-B4 FRH3 (Kabat) | KATLTVDKSISTAYMELSRLRSDDTAVYYCAR |
| 71 | h2H8-B4 FRL1 (Kabat) | DIQMTQSPSSLSASVGDRVTITC |
| 72 | h2H8-B4 FRL2 (Kabat) | WYQQKPGKAPKLLIY |
| 73 | h2H8-B4 FRL3 (Kabat) | GVPSRFSGSGSGTDFTLTISSLQPEDFATYYC |
| 74 | Extracellular loop 2 of | YQVASEDGVLQCYSFYNQQTLKWKIFTNFKM |
| | CCR8 (residues 172- | |
| | 202) | |
| 75 | CCR8_5888 | MDYQVSSPIYDINYYTSEPCQKINVKQIAARLLLAVFYCLLFVFSLLGN |
| | | SLVILVLVVCKKLRSITDVYLLNLALSDLLFVFSFPFQTYYLLDQWVFG |
| | | TVMCKVVSGFYYIGFYSSMFFITLMSVDRYLAVVHAVYALKVRTIRMG |
| | | TTLCLAVWLTAIMATIPLLVFYQVASEDGVLQCYSFYNQQTLKWKIFTN |
| | | FKMNILGLLIPFTIFMFCYIKILHQLKRCQNHNKTKAIRLVLIVVIAS |
| | | LLFWVPFNVVLFLTSLHSMHILDGCSISQQLTYATHVTEIISFTHCCVN |
| | | PVIYAFVGEKFKKHLSEIFQKSCSQIFNYLGRQMPRESCEKSSSCQQ |
| | | HSSRSSSVDYIL |
| 76 | CCR8_8588 | MDYTLDLSVTTVTDYYYPDIFSSPCDAELIQTNGKLLLAVFYCLLFVF |
| | | SLLGNSLVILVLVVCKKLRSITDVYLLNLALSDLLFVFSFPFQTYYAAA |
| | | QWDFGNTMCQVVSGFYYIGFYSSMFFITLMSVDRYLAVVHAVYALK |
| | | VRTIRMGTTLCLAVWLTAIMATIPLLVFYQVASEDGVLQCYSFYNQQ |
| | | TLKWKIFTNFKMNILGLLIPFTIFMFCYIKILHQLKRCQNHNKTKAIRLV |
| | | LIVVIASLLFWVPFNVVLFLTSLHSMHILDGCSISQQLTYATHVTEIISF |
| | | THCCVNPVIYAFVGEKFKKHLSEIFQKSCSQIFNYLGRQMPRESCEK |
| | | SSSCQQHSSRSSSVDYIL |
| 77 | CCR8_8858 | MDYTLDLSVTTVTDYYYPDIFSSPCDAELIQTNGKLLLAVFYCLLFVF |
| | | SLLGNSLVILVLVVCKKLRSITDVYLLNLALSDLLFVFSFPFQTYYLLD |
| | | QWVFGTVMCKVVSGFYYIGFYSSMFFITLMSVDRYLAVVHAVYALK |
| | | VRTIRMGTTLCLAVWLTAIMATIPLLVFTRSQKEGLHYTCSSHFPYSQ |
| | | YQFWKNFQTLKINILGLLIPFTIFMFCYIKILHQLKRCQNHNKTKAIRLV |
| | | LIVVIASLLFWVPFNVVLFLTSLHSMHILDGCSISQQLTYATHVTEIISF |
| | | THCCVNPVIYAFVGEKFKKHLSEIFQKSCSQIFNYLGRQMPRESCEK |
| | | SSSCQQHSSRSSSVDYIL |
| 78 | CCR8_8885 | MDYTLDLSVTTVTDYYYPDIFSSPCDAELIQTNGKLLLAVFYCLLFVF |
| | | SLLGNSLVILVLVVCKKLRSITDVYLLNLALSDLLFVFSFPFQTYYLLD |
| | | QWVFGTVMCKVVSGFYYIGFYSSMFFITLMSVDRYLAVVHAVYALK |
| | | VRTIRMGTTLCLAVWLTAIMATIPLLVFYQVASEDGVLQCYSFYNQQ |
| | | TLKWKIFTNFKMNILGLLIPFTIFMFCYIKILHQLKRCQNHNKTKAIRLV |
| | | LIVVIASLLFWVPFNVVLFLTSLQEFFGLNNCSSSNRLDQATHVTEIIS |
| | | FTHCCVNPVIYAFVGEKFKKHLSEIFQKSCSQIFNYLGRQMPRESCE |
| | | KSSSCQQHSSRSSSVDYIL |
| 79 | hCCR8_172-173 YQ- | MDYTLDLSVTTVTDYYYPDIFSSPCDAELIQTNGKLLLAVFYCLLFVF |
| | AA | SLLGNSLVILVLVVCKKLRSITDVYLLNLALSDLLFVFSFPFQTYYLLD |
| | | QWVFGTVMCKVVSGFYYIGFYSSMFFITLMSVDRYLAVVHAVYALK |
| | | VRTIRMGTTLCLAVWLTAIMATIPLLVFAAVASEDGVLQCYSFYNQQ |
| | | TLKWKIFTNFKMNILGLLIPFTIFMFCYIKILHQLKRCQNHNKTKAIRLV |
| | | LIVVIASLLFWVPFNVVLFLTSLHSMHILDGCSISQQLTYATHVTEIISF |
| | | THCCVNPVIYAFVGEKFKKHLSEIFQKSCSQIFNYLGRQMPRESCEK |
| | | SSSCQQHSSRSSSVDYIL |
| 80 | hCCR8_174-175 VA- | MDYTLDLSVTTVTDYYYPDIFSSPCDAELIQTNGKLLLAVFYCLLFVF |
| | AG | SLLGNSLVILVLVVCKKLRSITDVYLLNLALSDLLFVFSFPFQTYYLLD |
| | | QWVFGTVMCKVVSGFYYIGFYSSMFFITLMSVDRYLAVVHAVYALK |
| | | VRTIRMGTTLCLAVWLTAIMATIPLLVFYQAGSEDGVLQCYSFYNQQ |
| | | TLKWKIFTNFKMNILGLLIPFTIFMFCYIKILHQLKRCQNHNKTKAIRLV |
| | | LIVVIASLLFWVPFNVVLFLTSLHSMHILDGCSISQQLTYATHVTEIISF |
| | | THCCVNPVIYAFVGEKFKKHLSEIFQKSCSQIFNYLGRQMPRESCEK |
| | | SSSCQQHSSRSSSVDYIL |
| 81 | hCCR8_176-177 SE- | MDYTLDLSVTTVTDYYYPDIFSSPCDAELIQTNGKLLLAVFYCLLFVF |
| | AA | SLLGNSLVILVLVVCKKLRSITDVYLLNLALSDLLFVFSFPFQTYYLLD |
| | | QWVFGTVMCKVVSGFYYIGFYSSMFFITLMSVDRYLAVVHAVYALK |
| | | VRTIRMGTTLCLAVWLTAIMATIPLLVFYQVAAADGVLQCYSFYNQQ |
| | | TLKWKIFTNFKMNILGLLIPFTIFMFCYIKILHQLKRCQNHNKTKAIRLV |
| | | LIVVIASLLFWVPFNVVLFLTSLHSMHILDGCSISQQLTYATHVTEIISF |
| | | THCCVNPVIYAFVGEKFKKHLSEIFQKSCSQIFNYLGRQMPRESCEK |
| | | SSSCQQHSSRSSSVDYIL |
| 82 | hCCR8_178-179 DG- | MDYTLDLSVTTVTDYYYPDIFSSPCDAELIQTNGKLLLAVFYCLLFVF |
| | AA | SLLGNSLVILVLVVCKKLRSITDVYLLNLALSDLLFVFSFPFQTYYLLD |
| | | QWVFGTVMCKVVSGFYYIGFYSSMFFITLMSVDRYLAVVHAVYALK |
| | | VRTIRMGTTLCLAVWLTAIMATIPLLVFYQVASEAAVLQCYSFYNQQT |
| | | LKWKIFTNFKMNILGLLIPFTIFMFCYIKILHQLKRCQNHNKTKAIRLVLI |
| | | VVIASLLFWVPFNVVLFLTSLHSMHILDGCSISQQLTYATHVTEIISFT |
| | | HCCVNPVIYAFVGEKFKKHLSEIFQKSCSQIFNYLGRQMPRESCEKS |
| | | SSCQQHSSRSSSVDYIL |
| 83 | h2H8_VH | QVQLVQSGAEVKKPGASVKVSCKASGYTFTDYNMHWVRQAPGQG |
| | | LEWIGYVNPNNGGTSYNQKFKGKATLTVDKSISTAYMELSRLRSDD |
| | | TAVYYCARWVLRRNFAYWGQGTLVTVSS |
| 84 | H2H8_VK | DIQMTQSPSSLSASVGDRVTITCKASQDVNTAVAWYQQKPGKAPKL |
| | | LIYWASTRHTGVPSRFSGSGSGTDFTLTISSLQPEDFATYYCQQHY |
| | | RNPYTFGQGTKLEIK |

### DETAILED DESCRIPTION OF THE EMBODIMENTS

Further aspects of the present invention and further embodiments of the aspects described in the preceding paragraphs will become apparent from the following description, given by way of example and with reference to the accompanying drawings.

Reference will now be made in detail to certain embodiments of the invention. While the invention will be described in conjunction with the embodiments, it will be understood that the intention is not to limit the invention to those embodiments. On the contrary, the invention is intended to cover all alternatives, modifications, and equivalents, which may be included within the scope of the present invention as defined by the claims.

Various antigen binding proteins that bind to CCR8 are known but lack one or more properties that make them suitable for preclinical or clinical development. The CCR8 binding antigen binding proteins described herein display the advantageous property of binding not only to human CCR8 but also to mouse CCR8 and cynomolgus (cyno) CCR8. This provides the opportunity to explore the properties of the antigen binding proteins in both mouse and cynomolgus preclinical models. Further, the antigen binding proteins also display specificity for CCR8 as they do not bind to a number of closely related receptors (as shown herein). Surprisingly, the CCR8 antigen binding protein as described in the Examples that binds to human, mouse and cynomolgus CCR8 was generated by immunisation of a mouse with mouse CCR8.

Further, a CCR8 antibody generated was then humanised and surprisingly the binding affinity to human CCR8 was retained as was the ability to bind to live cells.

### General and Definitions

Throughout this specification, unless specifically stated otherwise or the context requires otherwise, reference to a single step, composition of matter, group of steps or group of compositions of matter shall be taken to encompass one and a plurality (i.e. one or more) of those steps, compositions of matter, groups of steps or groups of compositions of matter. Thus, as used herein, the singular forms “a”, “an” and “the” include plural aspects, and vice versa, unless the context clearly dictates otherwise. For example, reference to “a” includes a single as well as two or more; reference to “an” includes a single as well as two or more; reference to “the” includes a single as well as two or more and so forth.

Those skilled in the art will appreciate that the present invention is susceptible to variations and modifications other than those specifically described. It is to be understood that the invention includes all such variations and modifications. The invention also includes all the steps, features, compositions and compounds referred to or indicated in this specification, individually or collectively, and any and all combinations or any two or more of said steps or features.

One skilled in the art will recognise many methods and materials similar or equivalent to those described herein, which could be used in the practice of the present invention. The present invention is in no way limited to the methods and materials described.

All of the patents and publications referred to herein are incorporated by reference in their entirety.

The present invention is not to be limited in scope by the specific examples described herein, which are intended for the purpose of exemplification only. Functionally-equivalent products, compositions and methods are clearly within the scope of the present invention.

For purposes of interpreting this specification, the following definitions will apply and whenever appropriate, terms used in the singular will also include the plural and vice versa. In the event that any definition set forth conflicts with any document incorporated herein by reference, the definition set forth below shall prevail.

Unless specifically defined otherwise, all technical and scientific terms used herein shall be taken to have the same meaning as commonly understood by one of ordinary skill in the art (for example, in cell culture, molecular genetics, immunology, immunohistochemistry, protein chemistry, and biochemistry).

Unless otherwise indicated, the recombinant protein, cell culture, and immunological techniques utilised in the present disclosure are standard procedures, well known to those skilled in the art. Such techniques are described and explained throughout the literature in sources such as, J. Perbal, A Practical Guide to Molecular Cloning, John Wiley and Sons (1984), J. Sambrook et al. Molecular Cloning: A Laboratory Manual, Cold Spring Harbour Laboratory Press (1989), T. A. Brown (editor), Essential Molecular Biology: A Practical Approach, Volumes 1 and 2, IRL Press (1991), D. M. Glover and B. D. Hames (editors), DNA Cloning: A Practical Approach, Volumes 1-4, IRL Press (1995 and 1996), and F. M. Ausubel et al. (editors), Current Protocols in Molecular Biology, Greene Pub. Associates and Wiley-Interscience (1988, including all updates until present), Ed Harlow and David Lane (editors) Antibodies: A Laboratory Manual, Cold Spring Harbour Laboratory, (1988), and J. E. Coligan et al. (editors) Current Protocols in Immunology, John Wiley & Sons (including all updates until present).

The description and definitions of variable regions and parts thereof, immunoglobulins, antibodies and fragments thereof herein may be further clarified by the discussion in Kabat Sequences of Proteins of Immunological Interest, National Institutes of Health, Bethesda, Md., 1987 and 1991, Bork et al., J Mol. Biol. 242, 309-320, 1994, Chothia and Lesk J. Mol Biol. 196:901-917, 1987, Chothia et al. Nature 342, 877-883, 1989, Martin (“enhanced Chothia”; Mol Immunol. (2008) 45:3832-9; and/or or Al-Lazikani et al., J Mol Biol 273, 927-948, 1997.

The term “and/or”, e.g., “X and/or Y” shall be understood to mean either “X and Y” or “X or Y” and shall be taken to provide explicit support for both meanings or for either meaning.

As used herein the term “derived from” shall be taken to indicate that a specified integer may be obtained from a particular source albeit not necessarily directly from that source.

As used herein, the term “CCR8” or “C-C chemokine receptor type 8” refers to a G-protein coupled receptor. CCR8 is known to have at least four ligands: CCL1, CCL8, CCL16, and CCL18. CCL1 is thought to potentiate human Treg cells by inducing CCR8, FOXp3, CD39, Granzyme B, and IL-10 expression, in a STAT3-dependent manner. See, e.g., Barsheshet et al., PNAS 114(23):6086-91 (Jun. 6, 2017). CCR8 is expressed primarily on Treg cells and to a lesser extent on small fractions of TH2 cells, monocytic cells, NK cells, and CD8+ cells. CCR8 is a transmembrane receptor having seven transmembrane domains, an extracellular N-terminal domain, and an intracellular C-terminal domain, which interacts with G-protein. Exemplary amino acid sequences for human CCR8 (SEQ ID NO: 55), mouse CCR8 (SEQ ID NO: 56) and cynomolgus CCR8 (SEQ ID NO: 57) are shown in Table 1.

The term “isolated protein” or “isolated polypeptide” is a protein or polypeptide that by virtue of its origin or source of derivation is not associated with naturally-associated components that accompany it in its native state; is substantially free of other proteins from the same source. A protein may be rendered substantially free of naturally associated components or substantially purified by isolation, using protein purification techniques known in the art. By “substantially purified” is meant the protein is substantially free of contaminating agents, e.g., at least about 70% or 75% or 80% or 85% or 90% or 95% or 96% or 97% or 98% or 99% free of contaminating agents.

The term “recombinant” shall be understood to mean the product of artificial genetic recombination. Accordingly, in the context of a recombinant protein comprising an antibody antigen binding domain, this term does not encompass an antibody naturally-occurring within a subject's body that is the product of natural recombination that occurs during B cell maturation. However, if such an antibody is isolated, it is to be considered an isolated protein comprising an antibody antigen binding domain. Similarly, if nucleic acid encoding the protein is isolated and expressed using recombinant means, the resulting protein is a recombinant protein comprising an antibody antigen binding domain. A recombinant protein also encompasses a protein expressed by artificial recombinant means when it is within a cell, tissue or subject, e.g., in which it is expressed.

The term “protein” shall be taken to include a single polypeptide chain, i.e., a series of contiguous amino acids linked by peptide bonds or a series of polypeptide chains covalently or non-covalently linked to one another (i.e., a polypeptide complex). For example, the series of polypeptide chains can be covalently linked using a suitable chemical or a disulphide bond. Examples of non-covalent bonds include hydrogen bonds, ionic bonds, Van der Waals forces, and hydrophobic interactions. The protein may include one or more non-natural amino acids.

The term “polypeptide” or “polypeptide chain” will be understood from the foregoing paragraph to mean a series of contiguous amino acids linked by peptide bonds.

As used herein, the term “antigen binding domain” and shall be taken to mean a region of an antibody that is capable of specifically binding to an antigen, i.e., a V_{H} or a V_{L} or an Fv comprising both a VH and a VL. The antigen binding domain need not be in the context of an entire antibody, e.g., it can be in another form, e.g., as described herein, such as a scFv.

For the purposes for the present disclosure, the term “antibody” includes a protein capable of specifically binding to one or a few closely related antigens by virtue of an antigen binding domain contained within a Fv. This term includes four chain antibodies (e.g., two light chains and two heavy chains), recombinant or modified antibodies (e.g., chimeric antibodies, humanised antibodies, human antibodies, CDR-grafted antibodies, primatised antibodies, de-immunised antibodies, synhumanised antibodies, half-antibodies, bispecific antibodies).

An antibody generally comprises constant domains, which can be arranged into a constant region or constant fragment or fragment crystallisable (Fc). Exemplary forms of antibodies comprise a four-chain structure as their basic unit. Full-length antibodies comprise two heavy chains (˜50 to 70 kD) covalently linked and two light chains (˜23 kDa each). A light chain generally comprises a variable region (if present) and a constant domain and in mammals is either a κ light chain or a λ light chain. A heavy chain generally comprises a variable region and one or two constant domain(s) linked by a hinge region to additional constant domain(s). Heavy chains of mammals are of one of the following types α, δ, ε, γ, or μ. Each light chain is also covalently linked to one of the heavy chains. For example, the two heavy chains and the heavy and light chains are held together by inter-chain disulfide bonds and by non-covalent interactions. The number of inter-chain disulfide bonds can vary among different types of antibodies. Each chain has an N-terminal variable region (V_{H} or V_{L} wherein each are ˜110 amino acids in length) and one or more constant domains at the C-terminus. The constant domain of the light chain (C_{L} which is ˜110 amino acids in length) is aligned with and disulfide bonded to the first constant domain of the heavy chain (C_{H1} which is 330 to 440 amino acids in length). The light chain variable region is aligned with the variable region of the heavy chain. The antibody heavy chain can comprise 2 or more additional C_{H} domains (such as, C_{H2}, C_{H3} and the like) and can comprise a hinge region between the C_{H1} and C_{H2} constant domains. Antibodies can be of any type (e.g., IgG, IgE, IgM, IgD, IgA, and IgY), class (e.g., IgG₁, IgG₂, IgG₃, IgG₄, IgA₁ and IgA₂) or subclass. In one example, the antibody is a murine (mouse or rat) antibody or a primate (such as, human) antibody. In one example the antibody heavy chain is missing a C-terminal lysine residue. In one example, the antibody is humanised, synhumanised, chimeric, CDR-grafted or deimmunised.

The terms “full-length antibody”, “intact antibody” or “whole antibody” are used interchangeably to refer to an antibody in its substantially intact form, as opposed to an antigen binding fragment of an antibody. Specifically, whole antibodies include those with heavy and light chains including an Fc region. The constant domains may be wild-type sequence constant domains (e.g., human wild-type sequence constant domains) or amino acid sequence variants thereof.

As used herein, “variable region” refers to the portions of the light and/or heavy chains of an antibody as defined herein that is capable of specifically binding to an antigen and, includes amino acid sequences of complementarity determining regions (CDRs); i.e., CDR₁, CDR₂, and CDR₃, and framework regions (FRs). For example, the variable region comprises three or four FRs (e.g., FR₁, FR₂, FR₃ and optionally FR4) together with three CDRs. V_{H} refers to the variable region of the heavy chain. V_{L} refers to the variable region of the light chain.

As used herein, the term “complementarity determining regions” (syn. CDRs; i.e., CDR₁, CDR₂, and CDR₃) refers to the amino acid residues of an antibody variable region the presence of which are major contributors to specific antigen binding. Each variable region domain (V_{H} or V_{L}) typically has three CDRs identified as CDR₁, CDR₂ and CDR₃. The CDRs of V_{H} are also referred to herein as CDR H₁, CDR H₂ and CDR H₃, respectively, wherein CDR H₁ corresponds to CDR 1 of V_{H}, CDR H₂ corresponds to CDR 2 of V_{H} and CDR H₃ corresponds to CDR 3 of V_{H}. Likewise, the CDRs of V_{L} are referred to herein as CDR L₁, CDR L₂ and CDR L₃, respectively, wherein CDR L₁ corresponds to CDR 1 of V_{L}, CDR L₂ corresponds to CDR 2 of V_{L} and CDR L₃ corresponds to CDR 3 of V_{L}. In one example, the amino acid positions assigned to CDRs and FRs are defined according to Kabat Sequences of Proteins of Immunological Interest, National Institutes of Health, Bethesda, Md., 1987 and 1991 (also referred to herein as “the Kabat numbering system”). In another example, the amino acid positions assigned to CDRs and FRs are defined according to the Enhanced Chothia Numbering Scheme (http://www.bioinfo.org.uk/mdex.html). The present invention is not limited to FRs and CDRs as defined by the Kabat numbering system, but includes all numbering systems, including the canonical numbering system or of Chothia and Lesk J. Mol. Biol. 196: 901-917, 1987; Chothia et al., Nature 342: 877-883, 1989; and/or AI-Lazikani et al., J. Mol. Biol. 273: 927-948, 1997; the numbering system of Honnegher and Plükthun J. Mol. Biol. 309: 657-670, 2001; or the IMGT system discussed in Giudicelli et al., Nucleic Acids Res. 25: 206-211 1997.

“Framework regions” (FRs) are those variable region residues other than the CDR residues. The FRs of VH are also referred to herein as FR H₁, FR H₂, FR H₃ and FR H₄, respectively, wherein FR H₁ corresponds to FR 1 of V_{H}, FR H₂ corresponds to FR 2 of V_{H}, FR H₃ corresponds to FR 3 of V_{H} and FR H₄ corresponds to FR 4 of V_{H}. Likewise, the FRs of V_{L} are referred to herein as FR L₁, FR L₂, FR L₃ and FR L₄, respectively, wherein FR L₁ corresponds to FR 1 of V_{L}, FR L₂ corresponds to FR 2 of V_{L}, FR L₃ corresponds to FR 3 of V_{L} and FR L₄ corresponds to FR 4 of V_{L}.

As used herein, the term “Fv” shall be taken to mean any protein, whether comprised of multiple polypeptides or a single polypeptide, in which a V_{L} and a V_{H} associate and form a complex having an antigen binding domain, i.e., capable of specifically binding to an antigen. The V_{H} and the V_{L} that form the antigen binding domain can be in a single polypeptide chain or in different polypeptide chains. Furthermore, an Fv of the invention (as well as any protein of the invention) may have multiple antigen binding domains that may or may not bind the same antigen. This term shall be understood to encompass fragments directly derived from an antibody as well as proteins corresponding to such a fragment produced using recombinant means. In some examples, the V_{H} is not linked to a heavy chain constant domain (C_{H}) 1 and/or the V_{L} is not linked to a light chain constant domain (C_{L}). Exemplary Fv containing polypeptides or proteins include a Fab fragment, a Fab′ fragment, a F(ab′) fragment, a scFv, a diabody, a triabody, a tetrabody or higher order complex, or any of the foregoing linked to a constant region or domain thereof, e.g., CH₂ or CH₃ domain, e.g., a minibody.

A “Fab fragment” consists of a monovalent antigen-binding fragment of an immunoglobulin and can be produced by digestion of a whole antibody with the enzyme papain, to yield a fragment consisting of an intact light chain and a portion of a heavy chain or can be produced using recombinant means. A “Fab′ fragment” of an antibody can be obtained by treating a whole antibody with pepsin, followed by reduction, to yield a molecule consisting of an intact light chain and a portion of a heavy chain comprising a V_{H} and a single constant domain. Two Fab′ fragments are obtained per antibody treated in this manner. A Fab′ fragment can also be produced by recombinant means. A “F(ab′)₂ fragment” of an antibody consists of a dimer of two Fab′ fragments held together by two disulfide bonds, and is obtained by treating a whole antibody molecule with the enzyme pepsin, without subsequent reduction. A “Fab2” fragment is a recombinant fragment comprising two Fab fragments linked using, for example a leucine zipper or a CH₃ domain. A “single chain Fv” or “scFv” is a recombinant molecule containing the variable region fragment (Fv) of an antibody in which the variable region of the light chain and the variable region of the heavy chain are covalently linked by a suitable, flexible polypeptide linker.

As used herein, the term “binds” in reference to the interaction of an antigen binding protein or an antigen binding domain thereof with an antigen means that the interaction is dependent upon the presence of a particular structure (e.g., an antigenic determinant or epitope) on the antigen. For example, an antibody recognizes and binds to a specific protein structure rather than to proteins generally. If an antibody binds to epitope “A”, the presence of a molecule containing epitope “A” (or free, unlabelled “A”), in a reaction containing labelled “A” and the protein, will reduce the amount of labelled “A” bound to the antibody.

As used herein, the term “specifically binds” or “binds specifically” shall be taken to mean that an antigen binding protein of the invention reacts or associates more frequently, more rapidly, with greater duration and/or with greater affinity with a particular antigen or cell expressing same than it does with alternative antigens or cells. For example, an antigen binding protein binds to CCR8 with materially greater affinity (e.g., 1.5 fold or 2 fold or 5 fold or 10 fold or 20 fold or 40 fold or 60 fold or 80 fold to 100 fold or 150 fold or 200 fold) than it does to other related molecules, such as other receptors described herein including CCR3 (preferably human CCR3), CCR6 (preferably human CCR6), and/or CCR7 (preferably human CCR7). In example of the present invention, an antigen binding protein that “specifically binds” to CCR8 with an affinity at least 1.5 fold or 2 fold or greater (e.g., 5 fold or 10 fold or 20 fold or 50 fold or 100 fold or 200 fold) than it does to the receptors described herein including CCR3 (preferably human CCR3), CCR6 (preferably human CCR6), and/or CCR7 (preferably human CCR7). Generally, but not necessarily, reference to binding means specific binding, and each term shall be understood to provide explicit support for the other term.

As used herein, the term “does not detectably bind” shall be understood to mean that an antigen binding protein, e.g. an antibody, binds to a candidate antigen at a level less than 10%, or 8% or 6% or 5% above background. The background can be the level of binding signal detected in the absence of the protein and/or in the presence of a negative control protein (e.g., an isotype control antibody) and/or the level of binding detected in the presence of a negative control antigen. The level of binding is detected using biosensor analysis (e.g. Biacore) in which the antigen binding protein is immobilised and contacted with an antigen.

As used herein, the term “does not significantly bind” shall be understood to mean that the level of binding of an antigen binding protein of the invention to a polypeptide is not statistically significantly higher than background, e.g., the level of binding signal detected in the absence of the antigen binding protein and/or in the presence of a negative control protein (e.g., an isotype control antibody) and/or the level of binding detected in the presence of a negative control polypeptide. The level of binding is detected using biosensor analysis (e.g. Biacore or Blitz) in which the antigen binding protein is immobilised and contacted with an antigen.

As used herein, the term “epitope” (syn. “antigenic determinant”) shall be understood to mean a region of CCR8 to which an antigen binding protein comprising an antigen binding domain of an antibody binds. Unless otherwise defined, this term is not necessarily limited to the specific residues or structure to which the antigen binding protein makes contact. For example, this term includes the region spanning amino acids contacted by the antigen binding protein and 5-10 (or more) or 2-5 or 1-3 amino acids outside of this region. In some examples, the epitope comprises a series of discontinuous amino acids that are positioned close to one another when antigen binding protein is folded, i.e., a “conformational epitope”. The skilled artisan will also be aware that the term “epitope” is not limited to peptides or polypeptides. For example, the term “epitope” includes chemically active surface groupings of molecules such as sugar side chains, phosphoryl side chains, or sulfonyl side chains, and, in certain examples, may have specific three-dimensional structural characteristics, and/or specific charge characteristics.

As used herein, the terms “preventing”, “prevent” or “prevention” include administering an antigen binding protein of the invention to thereby stop or hinder the development of at least one symptom of a condition. This term also encompasses treatment of a subject in remission to prevent or hinder relapse.

As used herein, the terms “treating”, “treat” or “treatment” include administering an antigen binding protein described herein to thereby reduce or eliminate at least one symptom of a specified disease or condition.

As used herein, the term “subject” shall be taken to mean any animal including humans, for example a mammal. Exemplary subjects include but are not limited to humans and non-human primates. For example, the subject is a human.

The terms “engineered cell” and “genetically modified cell” as used herein can be used interchangeably. The terms mean containing and/or expressing a foreign gene or nucleic acid sequence that in turn modifies the genotype or phenotype of the cell or its progeny.

### Antibodies

In one example, an antigen binding protein as described herein according to any example is an antibody.

Methods for generating antibodies are known in the art and/or described in Harlow and Lane (editors) Antibodies: A Laboratory Manual, Cold Spring Harbor Laboratory, (1988). Generally, in such methods CCR8 or a region thereof (e.g., an extracellular region) or immunogenic fragment or epitope thereof or a cell expressing and displaying same (i.e., an immunogen), optionally formulated with any suitable or desired carrier, adjuvant, or pharmaceutically acceptable excipient, is administered to a non-human animal, for example, a mouse, chicken, rat, rabbit, guinea pig, dog, horse, cow, goat or pig. The immunogen may be administered intranasally, intramuscularly, subcutaneously, intravenously, intradermally, intraperitoneally, or by other known route.

The production of polyclonal antibodies may be monitored by sampling blood of the immunised animal at various points following immunisation. One or more further immunisations may be given, if required to achieve a desired antibody titre. The process of boosting and titreing is repeated until a suitable titre is achieved. When a desired level of immunogenicity is obtained, the immunised animal is bled and the serum isolated and stored, and/or the animal is used to generate monoclonal antibodies (mAbs).

Monoclonal antibodies are one exemplary form of antibody contemplated by the present invention. The term “monoclonal antibody” or “mAb” refers to a homogeneous antibody population capable of binding to the same antigen(s), for example, to the same epitope within the antigen. This term is not intended to be limited with regard to the source of the antibody or the manner in which it is made.

For the production of mAbs any one of a number of known techniques may be used, such as, for example, the procedure exemplified in U.S. Pat. No. 4,196,265 or Harlow and Lane (1988), supra.

For example, a suitable animal is immunised with an immunogen under conditions sufficient to stimulate antibody producing cells. Rodents such as rabbits, mice and rats are exemplary animals. Mice genetically-engineered to express human antibodies, for example, which do not express murine antibodies, can also be used to generate an antibody of the present invention (e.g., as described in WO2002/066630).

Following immunisation, somatic cells with the potential for producing antibodies, specifically B lymphocytes (B cells), are selected for use in the mAb generating protocol. These cells may be obtained from biopsies of spleens, tonsils or lymph nodes, or from a peripheral blood sample. The B cells from the immunised animal are then fused with cells of an immortal myeloma cell, generally derived from the same species as the animal that was immunised with the immunogen.

Hybrids are amplified by culture in a selective medium comprising an agent that blocks the de novo synthesis of nucleotides in the tissue culture media. Exemplary agents are aminopterin, methotrexate and azaserine.

The amplified hybridomas are subjected to a functional selection for antibody specificity and/or titre, such as, for example, by flow cytometry and/or immunohistochemistry and/or immunoassay (e.g. radioimmunoassay, enzyme immunoassay, cytotoxicity assay, plaque assay, dot immunoassay, and the like).

Alternatively, ABL-MYC technology (NeoClone, Madison WI 53713, USA) is used to produce cell lines secreting mAbs (e.g., as described in Largaespada et al, J. Immunol. Methods. 197: 85-95, 1996).

Antibodies can also be produced or isolated by screening a display library, e.g., a phage display library, e.g., as described in U.S. Pat. No. 6,300,064 and/or U.S. Pat. No. 5,885,793.

The antibody of the present invention may be a synthetic antibody. For example, the antibody is a chimeric antibody, a humanised antibody, a synhumanised antibody, primatised antibody or a de-immunised antibody.

Several in vitro methods exist for determining the efficacy of antibodies n eliciting ADCC. Among these are chromium-51 [Cr51] release assays, europium [Eu] release assays, and sulfur-35 [S35] release assays. Usually, a labeled target cell line expressing a certain surface-exposed antigen is incubated with antibody specific for that antigen. After washing, effector cells expressing Fc receptor CD16 are typically co-incubated with the antibody-labeled target cells. Target cell lysis is subsequently typically measured by release of intracellular label, for instance by a scintillation counter or spectrophotometry. A preferred test is detailed in the Examples.

### Antibody Binding Domain-Containing Proteins

### Diabodies, Triabodies, Tetrabodies

In some examples, a protein of the invention is or comprises a diabody, triabody, tetrabody or higher order protein complex such as those described in WO98/044001 and/or WO94/007921.

For example, a diabody is a protein comprising two associated polypeptide chains, each polypeptide chain comprising the structure V_{L}-X-V_{H} or V_{H}-X-V_{L}, wherein V_{L} is an antibody light chain variable region, V_{H} is an antibody heavy chain variable region, X is a linker comprising insufficient residues to permit the V_{H} and V_{L} in a single polypeptide chain to associate (or form an Fv) or is absent, and wherein the VH of one polypeptide chain binds to a V_{L} of the other polypeptide chain to form an antigen binding domain, i.e., to form a Fv molecule capable of specifically binding to one or more antigens. The V_{L} and V_{H} can be the same in each polypeptide chain or the V_{L} and V_{H} can be different in each polypeptide chain so as to form a bispecific diabody (i.e., comprising two Fvs having different specificity).

Single Chain Fc (scFv)

The skilled artisan will be aware that scFvs comprise V_{H} and V_{L} regions in a single polypeptide chain and a polypeptide linker between the V_{H} and V_{L} which enables the scFv to form the desired structure for antigen binding (i.e., for the V_{H} and V_{L} of the single polypeptide chain to associate with one another to form a Fv). For example, the linker comprises in excess of 12 amino acid residues with (Gly4Ser)3 being one of the more favoured linkers for a scFv.

The present invention also contemplates a disulfide stabilised Fv (or diFv or dsFv), in which a single cysteine residue is introduced into a FR of V_{H} and a FR of V_{L} and the cysteine residues linked by a disulfide bond to yield a stable Fv.

Alternatively, or in addition, the present invention encompasses a dimeric scFv, i.e., a protein comprising two scFv molecules linked by a non-covalent or covalent linkage, e.g., by a leucine zipper domain (e.g., derived from Fos or Jun). Alternatively, two scFvs are linked by a peptide linker of sufficient length to permit both scFvs to form and to bind to an antigen, e.g., as described in US20060263367

### Other Antibodies and Proteins Comprising Antigen Binding Domains Thereof

The present invention also contemplates other antibodies and proteins comprising antigen-binding domains thereof, such as:
(i) “key and hole” bispecific proteins as described in U.S. Pat. No. 5,731,168;
(ii) heteroconjugate proteins, e.g., as described in U.S. Pat. No. 4,676,980;
(iii) heteroconjugate proteins produced using a chemical cross-linker, e.g., as described in U.S. Pat. No. 4,676,980;
(iv) Fab₃ (e.g., as described in EP19930302894).

In any of the aforementioned antibody architectures, the binding domains of the proteins may be joined via a linker. For example, in the context of an scFv, the linker between the VH and VL may be a combination of one or more amino acid residues so as to provide a flexible linker. The skilled person will be familiar with suitable linker sequences to utilise. In typical examples, the linker is comprised of one or more glycine residues and serine residues. In one example, the linker may comprise the sequence G₄S (i.e., GGGGS) and the like. The linker may also comprise repeats of glycine and serine residues, such as (G₄S)₃, although it will be appreciated that any variations thereon may also be suitable, such as (G₄S)₃T.

### Mutations to Proteins

The present invention also provides an antigen binding protein or a nucleic acid encoding same having at least 80% identity to a sequence disclosed herein. In one example, an antigen binding protein or nucleic acid of the invention comprises sequence at least about 85% or 90% or 95% or 97% or 98% or 99% identical to a sequence disclosed herein.

Alternatively, or additionally, the antigen binding protein comprises a CDR (e.g., three CDRs) at least about 80% or 85% or 90% or 95% or 97% or 98% or 99% identical to CDR(s) of a V_{H} or V_{L} as described herein according to any example.

In another example, a nucleic acid of the invention comprises a sequence at least about 80% or 85% or 90% or 95% or 97% or 98% or 99% identical to a sequence encoding an antigen binding protein having a function as described herein according to any example. The present invention also encompasses nucleic acids encoding an antigen binding protein of the invention, which differs from a sequence exemplified herein as a result of degeneracy of the genetic code.

The % identity of a nucleic acid or polypeptide is determined by GAP (Needleman and Wunsch. Mol. Biol. 48, 443-453, 1970) analysis (GCG program) with a gap creation penalty=5, and a gap extension penalty=0.3. The query sequence is at least 50 residues in length, and the GAP analysis aligns the two sequences over a region of at least 50 residues. For example, the query sequence is at least 100 residues in length and the GAP analysis aligns the two sequences over a region of at least 100 residues. For example, the two sequences are aligned over their entire length.

The present invention also contemplates a nucleic acid that hybridises under stringent hybridisation conditions to a nucleic acid encoding an antigen binding site described herein. A “moderate stringency” is defined herein as being a hybridisation and/or washing carried out in 2×SSC buffer, 0.1% (w/v) SDS at a temperature in the range 45° C. to 65° C., or equivalent conditions. A “high stringency” is defined herein as being a hybridisation and/or wash carried out in 0.1×SSC buffer, 0.1% (w/v) SDS, or lower salt concentration, and at a temperature of at least 65° C., or equivalent conditions. Reference herein to a particular level of stringency encompasses equivalent conditions using wash/hybridisation solutions other than SSC known to those skilled in the art. For example, methods for calculating the temperature at which the strands of a double stranded nucleic acid will dissociate (also known as melting temperature, or Tm) are known in the art. A temperature that is similar to (e.g., within 5° C. or within 10° C.) or equal to the Tm of a nucleic acid is considered to be high stringency. Medium stringency is to be considered to be within 10° C. to 20° C. or 10° C. to 15° C. of the calculated Tm of the nucleic acid.

The present invention also contemplates mutant forms of an antigen binding protein of the invention comprising one or more conservative amino acid substitutions compared to a sequence set forth herein. In some examples, the antigen binding protein comprises 10 or fewer, e.g., 9 or 8 or 7 or 6 or 5 or 4 or 3 or 2 or 1 conservative amino acid substitutions. A “conservative amino acid substitution” is one in which the amino acid residue is replaced with an amino acid residue having a similar side chain and/or hydropathicity and/or hydrophilicity.

Families of amino acid residues having similar side chains have been defined in the art, including basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), β-branched side chains (e.g., threonine, valine, isoleucine) and aromatic side chains (e.g., tyrosine, phenylalanine, tryptophan, histidine). Hydropathic indices are described, for example in Kyte and Doolittle J. Mol. Biol., 157: 105-132, 1982 and hydrophylic indices are described in, e.g., U.S. Pat. No. 4,554,101.

The present invention also contemplates non-conservative amino acid changes. For example, of particular interest are substitutions of charged amino acids with another charged amino acid and with neutral or positively charged amino acids. In some examples, the antigen binding protein comprises 10 or fewer, e.g., 9 or 8 or 7 or 6 or 5 or 4 or 3 or 2 or 1 non-conservative amino acid substitutions.

In one example, the mutation(s) occur within a FR of an antigen binding domain of an antigen binding protein of the invention. In another example, the mutation(s) occur within a CDR of an antigen binding protein of the invention.

Exemplary methods for producing mutant forms of an antigen binding protein include:
mutagenesis of DNA (Thie et al., Methods Mol. Biol. 525: 309-322, 2009) or RNA (Kopsidas et al., Immunol. Lett. 107:163-168, 2006; Kopsidas et al. BMC Biotechnology, 7: 18, 2007; and WO1999/058661);
introducing a nucleic acid encoding the polypeptide into a mutator cell, e.g., XL-1Red, XL-mutS and XL-mutS-Kanr bacterial cells (Stratagene);
DNA shuffling, e.g., as disclosed in Stemmer, Nature 370: 389-91, 1994; and
site directed mutagenesis, e.g., as described in Dieffenbach (ed) and Dveksler (ed) (In: PCR Primer: A Laboratory Manual, Cold Spring Harbor Laboratories, NY, 1995).

Exemplary methods for determining biological activity of the mutant antigen binding proteins of the invention will be apparent to the skilled artisan and/or described herein, e.g., antigen binding. For example, methods for determining antigen binding, competitive inhibition of binding, affinity, association, dissociation and therapeutic efficacy are described herein.

### Constant Regions

The present invention encompasses antigen binding proteins and/or antibodies described herein comprising a constant region of an antibody. This includes antigen binding fragments of an antibody fused to an Fc.

Sequences of constant regions useful for producing the proteins of the present invention may be obtained from a number of different sources. In some examples, the constant region or portion thereof of the protein is derived from a human antibody. The constant region or portion thereof may be derived from any antibody class, including IgM, IgG, IgD, IgA and IgE, and any antibody isotype, including IgG₁, IgG₂, IgG₃ and IgG₄.

In one example, the Fc region of the constant region has an increased ability to induce effector function, e.g., compared to a native or wild-type human IgG₁ or IgG₃ Fc region. In one example, the effector function is antibody-dependent cell-mediated cytotoxicity (ADCC) and/or antibody-dependent cell-mediated phagocytosis (ADCP) and/or complement-dependent cytotoxicity (CDC). Methods for assessing the level of effector function of an Fc region containing protein are known in the art and/or described herein.

### Additional Modifications

The present invention also contemplates additional modifications to an antibody or antigen binding protein comprising an Fc region or constant region.

For example, the antibody comprises one or more amino acid substitutions that increase the half-life of the protein. For example, the antibody comprises a Fc region comprising one or more amino acid substitutions that increase the affinity of the Fc region for the neonatal Fc region (FcRn). For example, the Fc region has increased affinity for FcRn at lower pH, e.g., about pH 6.0, to facilitate Fc/FcRn binding in an endosome. In one example, the Fc region has increased affinity for FcRn at about pH 6 compared to its affinity at about pH 7.4, which facilitates the re-release of Fc into blood following cellular recycling. These amino acid substitutions are useful for extending the half-life of a protein, by reducing clearance from the blood.

Exemplary amino acid substitutions include T250Q and/or M428L or T252A, T254S and T266F or M252Y, S254T and T256E or H433K and N434F according to the EU numbering system. Additional or alternative amino acid substitutions are described, for example, in US20070135620 or U.S. Pat. No. 7,083,784.

### Protein Production

In one example, an antigen binding protein described herein according to any example is produced by culturing a hybridoma under conditions sufficient to produce the protein, e.g., as described herein and/or as is known in the art.

### Recombinant Expression

In another example an antigen binding protein described herein according to any example is recombinant.

In the case of a recombinant protein, nucleic acid encoding same can be cloned into expression constructs or vectors, which are then transfected into host cells, such as E. coli cells, yeast cells, insect cells, or mammalian cells, such as simian COS cells, Chinese Hamster Ovary (CHO) cells, human embryonic kidney (HEK) cells, or myeloma cells that do not otherwise produce the protein. Exemplary cells used for expressing a protein are CHO cells, myeloma cells or HEK cells. Molecular cloning techniques to achieve these ends are known in the art and described, for example in Ausubel et al., (editors), Current Protocols in Molecular Biology, Greene Pub. Associates and Wiley-Interscience (1988, including all updates until present) or Sambrook et al., Molecular Cloning: A Laboratory Manual, Cold Spring Harbor Laboratory Press (1989). A wide variety of cloning and in vitro amplification methods are suitable for the construction of recombinant nucleic acids. Methods of producing recombinant antibodies are also known in the art, see, e.g., U.S. Pat. No. 4,816,567 or U.S. Pat. No. 5,530,101.

Following isolation, the nucleic acid is inserted operably linked to a promoter in an expression construct or expression vector for further cloning (amplification of the DNA) or for expression in a cell-free system or in cells.

As used herein, the term “promoter” is to be taken in its broadest context and includes the transcriptional regulatory sequences of a genomic gene, including the TATA box or initiator element, which is required for accurate transcription initiation, with or without additional regulatory elements (e.g., upstream activating sequences, transcription factor binding sites, enhancers and silencers) that alter expression of a nucleic acid, e.g., in response to a developmental and/or external stimulus, or in a tissue specific manner. In the present context, the term “promoter” is also used to describe a recombinant, synthetic or fusion nucleic acid, or derivative which confers, activates or enhances the expression of a nucleic acid to which it is operably linked. Exemplary promoters can contain additional copies of one or more specific regulatory elements to further enhance expression and/or alter the spatial expression and/or temporal expression of said nucleic acid.

As used herein, the term “operably linked to” means positioning a promoter relative to a nucleic acid such that expression of the nucleic acid is controlled by the promoter.

Many vectors for expression in cells are available. The vector components generally include, but are not limited to, one or more of the following: a signal sequence, a sequence encoding a protein (e.g., derived from the information provided herein), an enhancer element, a promoter, and a transcription termination sequence. The skilled artisan will be aware of suitable sequences for expression of a protein. Exemplary signal sequences include prokaryotic secretion signals (e.g., pelB, alkaline phosphatase, penicillinase, Ipp, or heat-stable enterotoxin II), yeast secretion signals (e.g., invertase leader, a factor leader, or acid phosphatase leader) or mammalian secretion signals (e.g., herpes simplex gD signal).

Exemplary promoters active in mammalian cells include cytomegalovirus immediate early promoter (CMV-IE), human elongation factor 1-α promoter (EF1), small nuclear RNA promoters (U1a and U1b), α-myosin heavy chain promoter, Simian virus 40 promoter (SV40), Rous sarcoma virus promoter (RSV), Adenovirus major late promoter, β-actin promoter; hybrid regulatory element comprising a CMV enhancer/β-actin promoter or an immunoglobulin promoter or active fragment thereof. Examples of useful mammalian host cell lines are monkey kidney CV1 line transformed by SV40 (COS-7, ATCC CRL 1651); human embryonic kidney line (293 or 293 cells subcloned for growth in suspension culture; baby hamster kidney cells (BHK, ATCC CCL 10); or Chinese hamster ovary cells (CHO).

Typical promoters suitable for expression in yeast cells such as for example a yeast cell selected from the group comprising Pichia pastoris, Saccharomyces cerevisiae and S. pombe, include, but are not limited to, the ADH1 promoter, the GAL1 promoter, the GAL4 promoter, the CUP1 promoter, the PHO5 promoter, the nmt promoter, the RPR1 promoter, or the TEF1 promoter.

Means for introducing the isolated nucleic acid or expression construct comprising same into a cell for expression are known to those skilled in the art. The technique used for a given cell depends on the known successful techniques. Means for introducing recombinant DNA into cells include microinjection, transfection mediated by DEAE-dextran, transfection mediated by liposomes such as by using lipofectamine (Gibco, MD, USA) and/or cellfectin (Gibco, MD, USA), PEG-mediated DNA uptake, electroporation and microparticle bombardment such as by using DNA-coated tungsten or gold particles (Agracetus Inc., WI, USA) amongst others.

The host cells used to produce the protein may be cultured in a variety of media, depending on the cell type used. Commercially available media such as Ham's FI0 (Sigma), Minimal Essential Medium ((MEM), (Sigma), RPMI-1640 (Sigma), and Dulbecco's Modified Eagle's Medium ((DMEM), Sigma) are suitable for culturing mammalian cells. Media for culturing other cell types discussed herein are known in the art.

### Isolation of Proteins

Methods for isolating a protein are known in the art and/or described herein.

Where an antigen binding protein is secreted into culture medium, supernatants from such expression systems can be first concentrated using a commercially available protein concentration filter, for example, an Amicon or Millipore Pellicon ultrafiltration unit. A protease inhibitor such as PMSF may be included in any of the foregoing steps to inhibit proteolysis and antibiotics may be included to prevent the growth of adventitious contaminants. Alternatively, or additionally, supernatants can be filtered and/or separated from cells expressing the protein, e.g., using continuous centrifugation.

The antigen binding protein prepared from the cells can be purified using, for example, ion exchange, hydroxyapatite chromatography, hydrophobic interaction chromatography, gel electrophoresis, dialysis, affinity chromatography (e.g., protein A affinity chromatography or protein G chromatography), or any combination of the foregoing. These methods are known in the art and described, for example in WO99/57134 or Ed Harlow and David Lane (editors) Antibodies: A Laboratory Manual, Cold Spring Harbor Laboratory, (1988).

The skilled artisan will also be aware that a protein can be modified to include a tag to facilitate purification or detection, e.g., a poly-histidine tag, e.g., a hexa-histidine tag, or an influenza virus hemagglutinin (HA) tag, or a Simian Virus 5 (V5) tag, or a FLAG tag, or a glutathione S-transferase (GST) tag. The resulting protein is then purified using methods known in the art, such as, affinity purification. For example, a protein comprising a hexa-his tag is purified by contacting a sample comprising the protein with nickel-nitrilotriacetic acid (Ni-NTA) that specifically binds a hexa-his tag immobilised on a solid or semi-solid support, washing the sample to remove unbound protein, and subsequently eluting the bound protein. Alternatively, or in addition a ligand or antibody that binds to a tag is used in an affinity purification method.

### Assaying Binding of Antigen Binding Proteins

It will be apparent to the skilled artisan that antigen binding protein of the present invention bind to CCR8. Methods for assessing binding to a protein are known in the art, e.g., as described in Scopes (In: Protein purification: principles and practice, Third Edition, Springer Verlag, 1994). Such a method generally involves immobilising the antigen binding protein and contacting it with labelled antigen (CCR8). Following washing to remove non-specific bound protein, the amount of label and, as a consequence, bound antigen is detected. Of course, the antigen binding protein can be labelled and the antigen immobilised. Panning-type assays can also be used. Alternatively, or additionally, surface plasmon resonance assays can be used.

Optionally, the dissociation constant (Kd), association constant (Ka) and/or affinity constant (KD) of an immobilised antigen binding protein for CCR8 or an epitope thereof is determined. The “Kd” or “Ka” or “KD” for a CCR8-binding protein is in one example measured by a radiolabelled or fluorescently-labelled CCR8 ligand binding assay. In the case of a “Kd”, this assay equilibrates the antigen binding protein with a minimal concentration of labelled CCR8 or epitope thereof in the presence of a titration series of unlabelled CCR8. Following washing to remove unbound CCR8 or epitope thereof, the amount of label is determined, which is indicative of the Kd of the protein.

According to another example the K_{d}, Kₐ or K_{D} is measured by using surface plasmon resonance assays, e.g., using BIAcore surface plasmon resonance (BIAcore, Inc., Piscataway, NJ) with immobilised dysfunctional P2X₇ receptor or a region thereof or immobilised antigen binding protein.

### Conditions to be Treated

The antigen binding proteins of the invention have particularly utility in the manufacture of medicaments (e.g., antibodies, antibody-drug conjugates), for the treatment of cancer.

Examples of cancers which may be treated according to the methods of the present invention include, pre-neoplastic and neoplastic diseases. Broad examples include breast tumours, colorectal tumours, adenocarcinomas, mesothelioma, bladder tumours, prostate tumours, germ cell tumour, hepatoma/cholongio, carcinoma, neuroendocrine tumours, pituitary neoplasm, small round cell tumour, squamous cell cancer, melanoma, atypical fibroxanthoma, seminomas, nonseminomas, stromal leydig cell tumours, Sertoli cell tumours, skin tumours, kidney tumours, testicular tumours, brain tumours, ovarian tumours, stomach tumours, oral tumours, bladder tumours, bone tumours, cervical tumours, esophageal tumours, laryngeal tumours, liver tumours, lung tumours, vaginal tumours and Wilm's tumour.

Examples of particular cancers include but are not limited to adenocarcinoma, adenoma, adenofibroma, adenolymphoma, adontoma, AIDS related cancers, acoustic neuroma, acute lymphocytic leukaemia, acute myeloid leukaemia, adenocystic carcinoma, adrenocortical cancer, agnogenic myeloid metaplasia, alopecia, alveolar soft-part sarcoma, ameloblastoma, angiokeratoma, angiolymphoid hyperplasia with eosinophilia, angioma sclerosing, angiomatosis, apudoma, anal cancer, angiosarcoma, aplastic anaemia, astrocytoma, ataxia-telangiectasia, basal cell carcinoma (skin), bladder cancer, bone cancers, bowel cancer, brain stem glioma, brain and CNS tumours, breast cancer, branchioma, CNS tumours, carcinoid tumours, cervical cancer, childhood brain tumours, childhood cancer, childhood leukaemia, childhood soft tissue sarcoma, chondrosarcoma, choriocarcinoma, chronic lymphocytic leukaemia, chronic myeloid leukaemia, colorectal cancers, cutaneous T-cell lymphoma, carcinoma (e.g. Walker, basal cell, basosquamous, Brown-Pearce, ductal, Ehrlich tumour, Krebs 2, Merkel cell, mucinous, non-small cell lung, oat cell, papillary, scirrhous, bronchiolar, bronchogenic, squamous cell, and transitional cell), carcinosarcoma, cervical dysplasia, cystosarcoma phyllodies, cementoma, chordoma, choristoma, chondrosarcoma, chondroblastoma, craniopharyngioma, cholangioma, cholesteatoma, cylindroma, cystadenocarcinoma, cystadenoma, dermatofibrosarcoma-protuberans, desmoplastic-small-round-cell-tumour, ductal carcinoma, dysgerminoam, endocrine cancers, endometrial cancer, ependymoma, esophageal cancer, Ewing's sarcoma, extra-hepatic bile duct cancer, eye cancer, eye: melanoma, retinoblastoma, fallopian tube cancer, fanconi anaemia, fibroma, fibrosarcoma, gall bladder cancer, gastric cancer, gastrointestinal cancers, gastrointestinal-carcinoid-tumour, genitourinary cancers, germ cell tumours, gestationaltrophoblastic-disease, glioma, gynaecological cancers, giant cell tumours, ganglioneuroma, glioma, glomangioma, granulosa cell tumour, gynandroblastoma, haematological malignancies, hairy cell leukaemia, head and neck cancer, hepatocellular cancer, hereditary breast cancer, histiocytosis, Hodgkin's disease, human papillomavirus, hydatidiform mole, hypercalcemia, hypopharynx cancer, hamartoma, hemangioendothelioma, hemangioma, hemangiopericytoma, hemangiosarcoma, hemangiosarcoma, histiocytic disorders, histiocytosis malignant, histiocytoma, hepatoma, hidradenoma, chondrosarcoma, immunoproliferative small, opoma, intraocular melanoma, islet cell cancer, Kaposi's sarcoma, kidney cancer, langerhan's cell-histiocytosis, laryngeal cancer, leiomyosarcoma, leukaemia, Li-fraumeni syndrome, lip cancer, liposarcoma, liver cancer, lung cancer, lymphedema, lymphoma, Hodgkin's lymphoma, non-Hodgkin's lymphoma, leigomyosarcoma, leukaemia (e.g. b-cell, mixed cell, null-cell, t-cell, t-cell chronic, htlv-ii-associated, lymphangiosarcoma, lymphocytic acute, lymphocytic chronic, mast-cell and myeloid), leukosarcoma, leydig cell tumour, liposarcoma, leiomyoma, leiomyosarcoma, lymphangioma, lymphangiocytoma, lymphagioma, lymphagiomyoma, lymphangiosarcoma, male breast cancer, malignant-rhabdoid-tumour-of-kidney, medulloblastoma, melanoma, Merkel cell cancer, mesothelioma, metastatic cancer, mouth cancer, multiple endocrine neoplasia, mycosis fungoides, myelodysplastic syndromes, myeloma, myeloproliferative disorders, malignant carcinoid syndrome carcinoid heart disease, medulloblastoma, meningioma, melanoma, mesenchymoma, mesonephroma, mesothelioma, myoblastoma, myoma, myosarcoma, myxoma, myxosarcoma, nasal cancer, nasopharyngeal cancer, nephroblastoma, neuroblastoma, neurofibromatosis, Nijmegen breakage syndrome, non-melanoma skin cancer, non-small-cell-lung-cancer-(nsclc), neurilemmoma, neuroblastoma, neuroepithelioma, neurofibromatosis, neurofibroma, neuroma, neoplasms (e.g. bone, breast, digestive system, colorectal, liver), ocular cancers, oesophageal cancer, oral cavity cancer, oropharynx cancer, osteosarcoma, ostomy ovarian cancer, pancreas cancer, paranasal cancer, parathyroid cancer, parotid gland cancer, penile cancer, peripheral-neuroectodermal-tumours, pituitary cancer, polycythemia vera, prostate cancer, osteoma, osteosarcoma, ovarian carcinoma, papilloma, paraganglioma, paraganglioma nonchromaffin, pinealoma, plasmacytoma, protooncogene, rare-cancers-and-associated-disorders, renal cell carcinoma, retinoblastoma, rhabdomyosarcoma, Rothmund-Thomson syndrome, reticuloendotheliosis, rhabdomyoma, salivary gland cancer, sarcoma, schwannoma, Sezary syndrome, skin cancer, small cell lung cancer (sclc), small intestine cancer, soft tissue sarcoma, spinal cord tumours, squamous-cell-carcinoma-(skin), stomach cancer, synovial sarcoma, sarcoma (e.g. Ewing's experimental, Kaposi's and mast-cell sarcomas), Sertoli cell tumour, synovioma, testicular cancer, thymus cancer, thyroid cancer, transitional-cell-cancer-(bladder), transitional-cell-cancer-(renal-pelvis-/-ureter), trophoblastic cancer, teratoma, theca cell tumour, thymoma, trophoblastic tumour, urethral cancer, urinary system cancer, uroplakins, uterine sarcoma, uterus cancer, vaginal cancer, vulva cancer, Waldenstrom's-macroglobulinemia and Wilms' tumour.

Other diseases and conditions include various inflammatory conditions. Examples may include a proliferative component. Particular examples include acne, angina, arthritis, aspiration pneumonia, disease, empyema, gastroenteritis, inflammation, intestinal flu, nee, necrotising enterocolitis, pelvic inflammatory disease, pharyngitis, pid, pleurisy, raw throat, redness, rubor, sore throat, stomach flu and urinary tract infections, chronic inflammatory demyelinating polyneuropathy, chronic inflammatory demyelinating polyradiculoneuropathy, chronic inflammatory demyelinating polyneuropathy or chronic inflammatory demyelinating polyradiculoneuropathy.

### Compositions

In some examples, an antigen binding protein as described herein can be administered orally, parenterally, by inhalation spray, adsorption, absorption, topically, rectally, nasally, bucally, vaginally, intraventricularly, via an implanted reservoir in dosage formulations containing conventional non-toxic pharmaceutically acceptable carriers, or by any other convenient dosage form. The term “parenteral” as used herein includes subcutaneous, intravenous, intramuscular, intraperitoneal, intrathecal, intraventricular, intrasternal, and intracranial injection or infusion techniques.

Methods for preparing an antigen binding protein into a suitable form for administration to a subject (e.g. a pharmaceutical composition) are known in the art and include, for example, methods as described in Remington's Pharmaceutical Sciences (18th ed., Mack Publishing Co., Easton, Pa., 1990) and U.S. Pharmacopeia: National Formulary (Mack Publishing Company, Easton, Pa., 1984).

The pharmaceutical compositions of this invention are particularly useful for parenteral administration, such as intravenous administration or administration into a body cavity or lumen of an organ or joint. The compositions for administration will commonly comprise a solution of an antigen binding protein dissolved in a pharmaceutically acceptable carrier, for example an aqueous carrier. A variety of aqueous carriers can be used, e.g., buffered saline and the like. The compositions may contain pharmaceutically acceptable auxiliary substances as required to approximate physiological conditions such as pH adjusting and buffering agents, toxicity adjusting agents and the like, for example, sodium acetate, sodium chloride, potassium chloride, calcium chloride, sodium lactate and the like. The concentration of an antigen binding protein of the present invention in these formulations can vary widely, and will be selected primarily based on fluid volumes, viscosities, body weight and the like in accordance with the particular mode of administration selected and the patient's needs. Exemplary carriers include water, saline, Ringer's solution, dextrose solution, and 5% human serum albumin. Non-aqueous vehicles such as mixed oils and ethyl oleate may also be used. Liposomes may also be used as carriers. The vehicles may contain minor amounts of additives that enhance isotonicity and chemical stability, e.g., buffers and preservatives.

The antigen binding proteins of the present invention may be formulated for local or topical administration, such as for topical application to the skin or tissue requiring treatment. Formulations for topical administration typically comprise a topical vehicle combined with active agent(s), with or without additional optional components. The pharmaceutical compositions of the invention may be in the form of a spray, cream, gel, lotion or the like for topical administration.

Suitable topical vehicles and additional components are well known in the art, and it will be apparent that the choice of a vehicle will depend on the particular physical form and mode of delivery. Topical vehicles include organic solvents such as alcohols (for example, ethanol, iso-propyl alcohol or glycerine), glycols such as butylene, isoprene or propylene glycol, aliphatic alcohols such as lanolin, mixtures of water and organic solvents and mixtures of organic solvents such as alcohol and glycerine, lipid-based materials such as fatty acids, acylglycerols including oils such as mineral oil, and fats of natural or synthetic origin, phosphoglycerides, sphingolipids and waxes, protein-based materials such as collagen and gelatine, silicone-based materials (both nonvolatile and volatile), and hydrocarbon-based materials such as microsponges and polymer matrices.

A composition may further include one or more components adapted to improve the stability or effectiveness of the applied formulation, such as stabilising agents, suspending agents, emulsifying agents, viscosity adjusters, gelling agents, preservatives, antioxidants, skin penetration enhancers, moisturisers and sustained release materials. Examples of such components are described in Martindale—The Extra Pharmacopoeia (Pharmaceutical Press, London 1993) and Martin (ed.), Remington's Pharmaceutical Sciences. Formulations may comprise microcapsules, such as hydroxymethylcellulose or gelatine-microcapsules, liposomes, albumin microspheres, microemulsions, nanoparticles or nanocapsules.

A topical formulation may be prepared in a variety of physical forms including, for example, solids, pastes, creams, foams, lotions, gels, powders, aqueous liquids, emulsions, sprays and skin patches. The physical appearance and viscosity of such forms can be governed by the presence and amount of emulsifier(s) and viscosity adjuster(s) present in the formulation. Solids are generally firm and non-pourable and commonly are formulated as bars or sticks, or in particulate form. Solids can be opaque or transparent, and optionally can contain solvents, emulsifiers, moisturisers, emollients, fragrances, dyes/colorants, preservatives and other active ingredients that increase or enhance the efficacy of the final product. Creams and lotions are often similar to one another, differing mainly in their viscosity. Both lotions and creams may be opaque, translucent or clear and often contain emulsifiers, solvents, and viscosity adjusting agents, as well as moisturisers, emollients, fragrances, dyes/colorants, preservatives and other active ingredients that increase or enhance the efficacy of the final product.

Gels can be prepared with a range of viscosities, from thick or high viscosity to thin or low viscosity. These formulations, like those of lotions and creams, may also contain solvents, emulsifiers, moisturisers, emollients, fragrances, dyes/colorants, preservatives and other active ingredients that increase or enhance the efficacy of the final product. Liquids are thinner than creams, lotions, or gels, and often do not contain emulsifiers. Liquid topical products often contain solvents, emulsifiers, moisturisers, emollients, fragrances, dyes/colorants, preservatives and other active ingredients that increase or enhance the efficacy of the final product.

Emulsifiers for use in topical formulations include, but are not limited to, ionic emulsifiers, cetearyl alcohol, non-ionic emulsifiers like polyoxyethylene oleyl ether, PEG-40 stearate, ceteareth-12, ceteareth-20, ceteareth-30, ceteareth alcohol, PEG-100 stearate and glyceryl stearate. Suitable viscosity adjusting agents include, but are not limited to, protective colloids or nonionic gums such as hydroxyethylcellulose, xanthan gum, magnesium aluminium silicate, silica, microcrystalline wax, beeswax, paraffin, and cetyl palmitate. A gel composition may be formed by the addition of a gelling agent such as chitosan, methyl cellulose, ethyl cellulose, polyvinyl alcohol, polyquaterniums, hydroxyethylcellulose, hydroxypropylcellulose, hydroxypropylmethylcellulose, carbomer or ammoniated glycyrrhizinate. Suitable surfactants include, but are not limited to, nonionic, amphoteric, ionic and anionic surfactants. For example, one or more of dimethicone copolyol, polysorbate 20, polysorbate 40, polysorbate 60, polysorbate 80, lauramide DEA, cocamide DEA, and cocamide MEA, oleyl betaine, cocamidopropyl phosphatidyl PG-dimonium chloride, and ammonium laureth sulfate may be used within topical formulations.

Preservatives include, but are not limited to, antimicrobials such as methylparaben, propylparaben, sorbic acid, benzoic acid, and formaldehyde, as well as physical stabilisers and antioxidants such as vitamin E, sodium ascorbate/ascorbic acid and propyl gallate. Suitable moisturisers include, but are not limited to, lactic acid and other hydroxy acids and their salts, glycerine, propylene glycol, and butylene glycol. Suitable emollients include lanolin alcohol, lanolin, lanolin derivatives, cholesterol, petrolatum, isostearyl neopentanoate and mineral oils. Suitable fragrances and colours include, but are not limited to, FD&C Red No. 40 and FD&C Yellow No. 5. Other suitable additional ingredients that may be included in a topical formulation include, but are not limited to, abrasives, absorbents, anticaking agents, antifoaming agents, antistatic agents, astringents (such as witch hazel), alcohol and herbal extracts such as chamomile extract, binders/excipients, buffering agents, chelating agents, film forming agents, conditioning agents, propellants, opacifying agents, pH adjusters and protectants.

Typical modes of delivery for topical compositions include application using the fingers, application using a physical applicator such as a cloth, tissue, swab, stick or brush, spraying including mist, aerosol or foam spraying, dropper application, sprinkling, soaking, and rinsing. Controlled release vehicles can also be used, and compositions may be formulated for transdermal administration (for example, as a transdermal patch).

Upon formulation, an antigen binding protein of the present invention will be administered in a manner compatible with the dosage formulation and in such amount as is therapeutically/prophylactically effective. Formulations are easily administered in a variety of dosage forms, such as the type of injectable solutions described above, but other pharmaceutically acceptable forms are also contemplated, e.g., tablets, pills, capsules or other solids for oral administration, suppositories, pessaries, nasal solutions or sprays, aerosols, inhalants, liposomal forms and the like. Pharmaceutical “slow release” capsules or compositions may also be used. Slow release formulations are generally designed to give a constant drug level over an extended period and may be used to deliver an antigen binding protein of the present invention.

WO2002/080967 describes compositions and methods for administering aerosolized compositions comprising antibodies for the treatment of, e.g., asthma, which are also suitable for administration of an antigen binding protein of the present invention.

### Dosage and Timing of Administration

Suitable dosages of an antigen binding protein of the present invention will vary depending on the specific an antigen binding protein, the condition to be treated and/or the subject being treated. It is within the ability of a skilled physician to determine a suitable dosage, e.g., by commencing with a sub-optimal dosage and incrementally modifying the dosage to determine an optimal or useful dosage. Alternatively, to determine an appropriate dosage for treatment/prophylaxis, data from the cell culture assays or animal studies are used, wherein a suitable dose is within a range of circulating concentrations that include the ED₅₀ of the active compound with little or no toxicity. The dosage may vary within this range depending upon the dosage form employed and the route of administration utilized. A therapeutically/prophylactically effective dose can be estimated initially from cell culture assays. A dose may be formulated in animal models to achieve a circulating plasma concentration range that includes the IC₅₀ (i.e., the concentration or amount of the compound which achieves a half-maximal inhibition of symptoms) as determined in cell culture. Such information can be used to more accurately determine useful doses in humans. Levels in plasma maybe measured, for example, by high performance liquid chromatography.

In some examples, a method of the present invention comprises administering a prophylactically or therapeutically effective amount of a protein described herein.

The term “therapeutically effective amount” is the quantity which, when administered to a subject in need of treatment, improves the prognosis and/or state of the subject and/or that reduces or inhibits one or more symptoms of a clinical condition described herein to a level that is below that observed and accepted as clinically diagnostic or clinically characteristic of that condition. The amount to be administered to a subject will depend on the particular characteristics of the condition to be treated, the type and stage of condition being treated, the mode of administration, and the characteristics of the subject, such as general health, other diseases, age, sex, genotype, and body weight. A person skilled in the art will be able to determine appropriate dosages depending on these and other factors. Accordingly, this term is not to be construed to limit the present invention to a specific quantity, e.g., weight or amount of protein(s), rather the present invention encompasses any amount of the antigen binding protein(s) sufficient to achieve the stated result in a subject.

As used herein, the term “prophylactically effective amount” shall be taken to mean a sufficient quantity of a protein to prevent or inhibit or delay the onset of one or more detectable symptoms of a clinical condition. The skilled artisan will be aware that such an amount will vary depending on, for example, the specific antigen binding protein(s) administered and/or the particular subject and/or the type or severity or level of condition and/or predisposition (genetic or otherwise) to the condition. Accordingly, this term is not to be construed to limit the present invention to a specific quantity, e.g., weight or amount of antigen binding protein(s), rather the present invention encompasses any amount of the antigen binding protein(s) sufficient to achieve the stated result in a subject.

### Kits

The present invention additionally comprises a kit comprising one or more of the following:
(i) an antigen binding protein of the invention or nucleic acid(s) or expression construct(s) encoding same;
(ii) a cell of the invention; or
(iii) a pharmaceutical composition of the invention.

In the case of a kit for detecting CCR8, the kit can additionally comprise a detection means, e.g., linked to an antigen binding protein of the invention.

In the case of a kit for therapeutic/prophylactic use, the kit can additionally comprise a pharmaceutically acceptable carrier.

Optionally a kit of the invention is packaged with instructions for use in a method described herein according to any example.

### EXAMPLES

### Example 1—Generation of Anti-Human CCR8 Antibodies by Immunising Mice with Human CCR8

mAbs reactive with human CCR8 were generated by immunizing C57BL/6 mice with 10⁷ L1.2 human CCR8 transfected cells, intraperitoneally (i.p), five to six times at 2-week intervals. The final immunization was injected intravenously (i.v). 4 days later, the spleen was removed and cells were fused with the SP2/0 cell line.

From 6 different fusions the inventors managed to isolated 6 clones specific for hCCR8. No clones demonstrated cross-reactivity for mouse or cyno CCR8.

### Example 2—Generation of Anti-Human CCR8 Antibodies by Immunising Mice with Mouse CCR8

mAbs reactive with mouse CCR8 were generated by immunizing C57BL/6 mice with 10⁷ L1.2 mouse CCR8 transfected cells, intraperitoneally (i.p), five to six times at 2-week intervals. The final immunization was injected intravenously (i.v). 4 days later, the spleen was removed and cells were fused with the SP2/0 cell line.

### Example 3—Isolation and Cross-Reactivity of Anti-CCR8 2H8 Antibody with Human, Mouse and Cyno CCR8

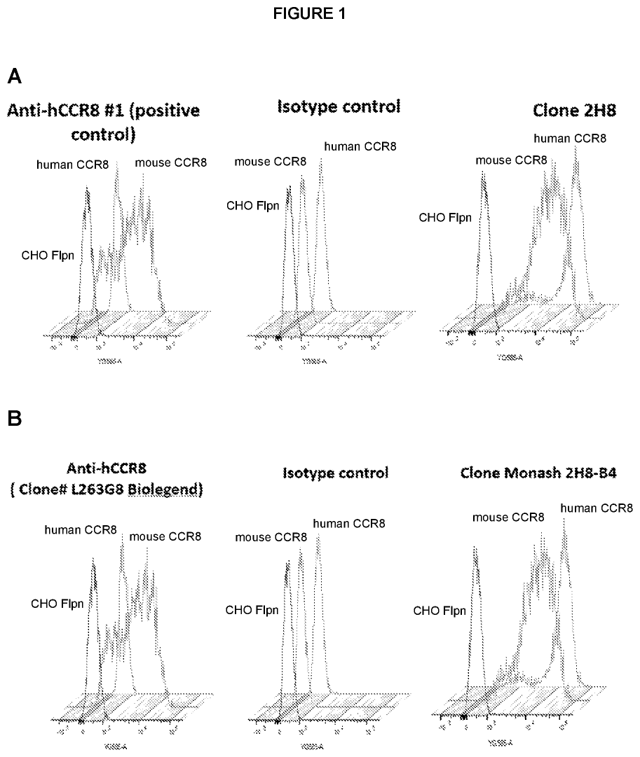

After a few immunisations the inventor(s) identified that the serum of the immunised mice contains antibodies able to bind both mouse CCR8 and human CCR8 (FIG. 1). The inventors performed a fusion on one of the mice and screened hybridomas by flow cytometry.

The inventor(s) isolated surrogate antibodies specific for mouse CCR8 but unexpectedly also found 1 clone (2H8; isotype IgG2b) showing reactivity for both human and mouse receptors (FIG. 1). This is in contrast to the antibodies obtained from Example 1 above, which only displayed reactivity to human CCR8 (FIG. 1A; anti-hCCR8 #1 presented as a representative example). Furthermore, the inventor(s) compared an existing anti-hCCR8 antibody (Clone #L263G8) from Biolegend which similarly only displayed reactivity to human CCR8 (FIG. 1B).

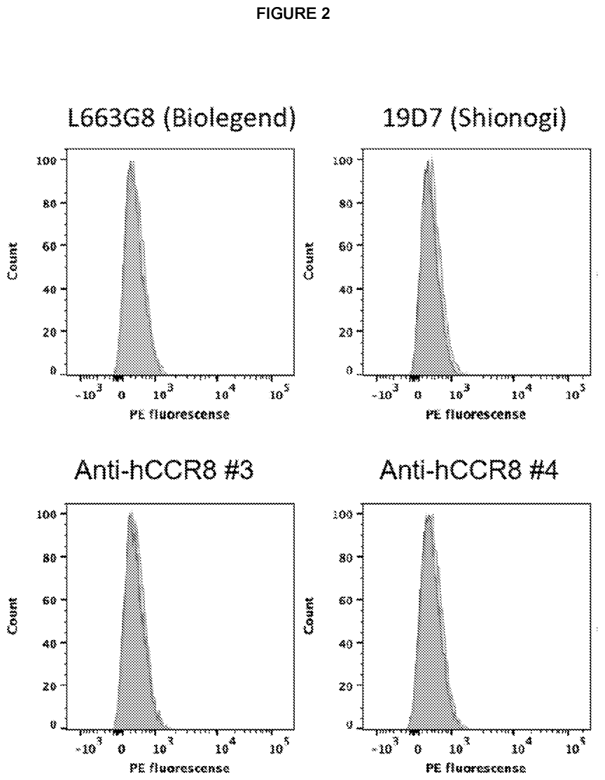

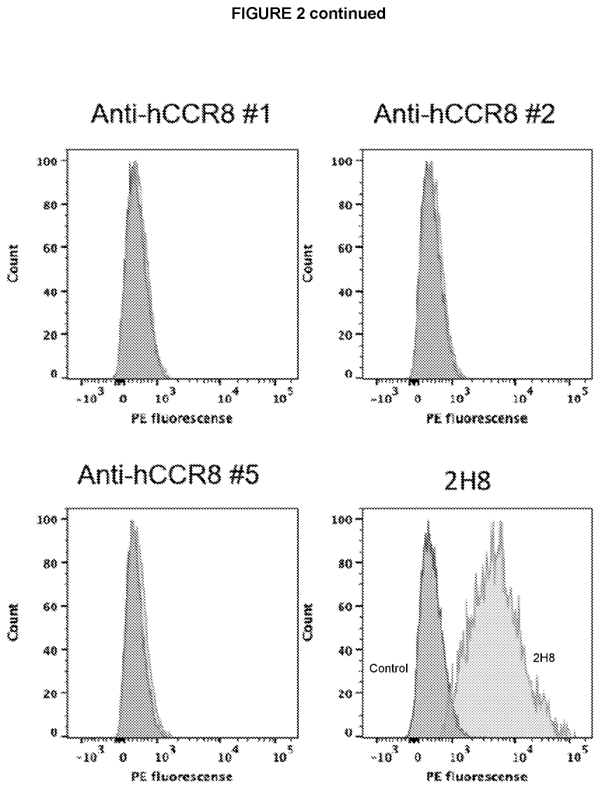

Next the inventor(s) tested the reactivity of the 2H8 antibody to cynomolgus monkey (cyno) CCR8. Surprisingly, 2H8 also displayed high reactivity to cyno CCR8 compared to isotype control (FIG. 2) while prior art antibodies and some generated using the method of Example 1 did not.

### Example 4—2H8 does not Cross-React with Other CCR Chemokine Receptors

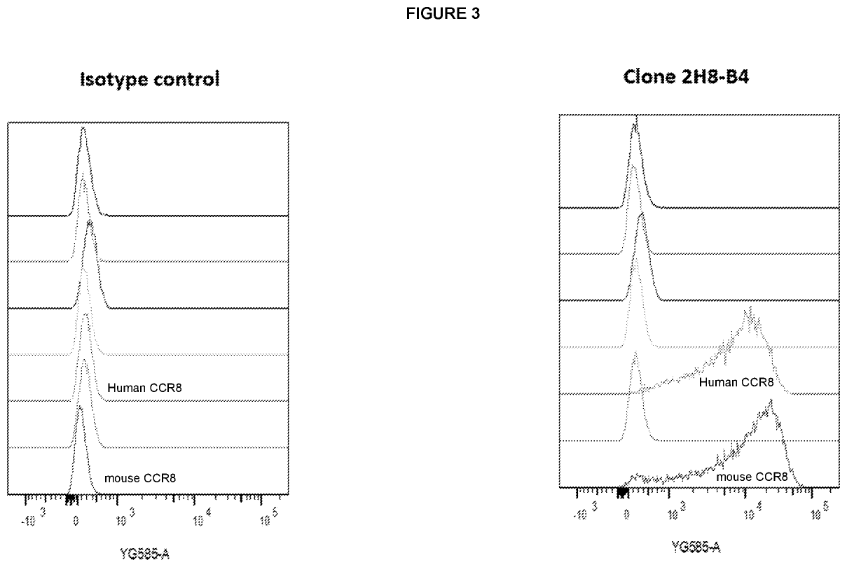

To confirm the specificity of 2H8 for CCR8, the inventor(s) tested the reactivity of 2H8 to other human CCR chemokine receptors and did not find any cross-reactivity to CCR chemokine receptors tested except for human and mouse CCR8 (FIG. 3).

### Example 5—2H8 Binds to Human Tumour Infiltrating Tregs (TiTregs)

To determine if 2H8 could bind to TiTregs the inventor(s) isolated a single cells suspension from a fresh colorectal tumour patient biopsy and stained the cells with anti-human CD3, anti-human CD4 and anti-human FoxP3 (TiTregs) and 2H8 or isotype control.

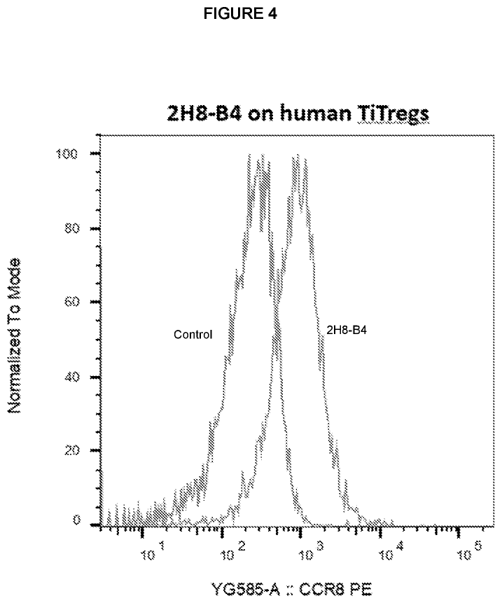

2H8 was found to bind to tumour infiltrating Tregs (CD3+; CD4+; FoxP3+ gate) while isotype control did not (FIG. 4).

### Example 6—Chimeric 2H8 Shows Similar Binding Affinity Compared to Existing Anti-hCCR8 Antibodies

The inventors generated a chimeric version of the 2H8 antibody comprising the heavy and light chain constant regions of human IgG1 as shown in SEQ ID NOs: 58 and 59.

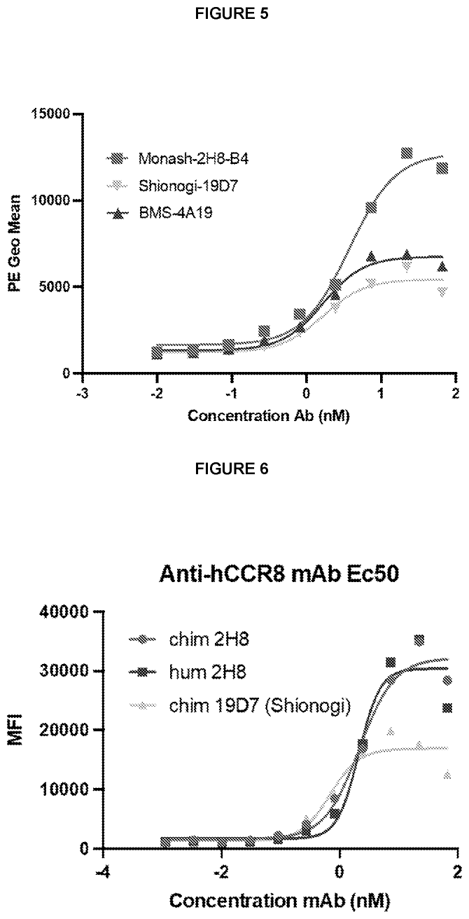

The inventor(s) used flow cytometry to compare the binding of the chimeric 2H8, Shionogi's 19D7, and BMS' 4A19 anti-hCCR8 antibodies to hCCR8-L1.2 transfected cells. All anti-hCCR8 antibodies displayed similar binding to hCCR8 (FIG. 5).

### Example 7—Humanised 2H8 Anti-CCR8 Antibodies

The inventor(s) next generated a humanised 2H8 antibody. Antibody humanization was performed by the CDR-grafting method.

Briefly, IMGT/V-QUEST and IMGT/Junctions analysis tools were used to identify human germline genes in which sequences from the variable regions of both the heavy and light chains were closely aligned with those of murine antibody 2H8. Framework sequences of these selected human germline genes were used as acceptor sequences for the 2H8 CDRs. However, murine residues were retained in the critical “Vernier” zone. The humanized VH and VL genes were synthesized by Genscript.

The binding activity of the humanised 2H8 antibody was compared to chimeric 2H8 and Shionogi's 19D7 anti-CCR8 antibodies using flow cytometry. The results as shown in FIG. 6 and Table 2 below.

TABLE 2
EC50 values for anti-CCR8 antibodies

| Clone | EC50 (nM) | Max MFI |
|---|---|---|
| Chimeric 2H8 | 2.1 | 34409 |
| Humanised 2H8 | 2.1 | 35243 |
| Chimeric 19D7 (Shionogi) | 0.9 | 19906 |

### Example 8—2H8 Binds to Human Tumour Infiltrating Tregs (TiTregs)

To determine if 2H8 could bind to TiTregs the inventor(s) isolated a single cells suspension from a fresh colorectal tumour patient biopsy and stained the cells with anti-human CD3, anti-human CD4 and anti-human FoxP3 (TiTregs) and 2H8 or isotype control.

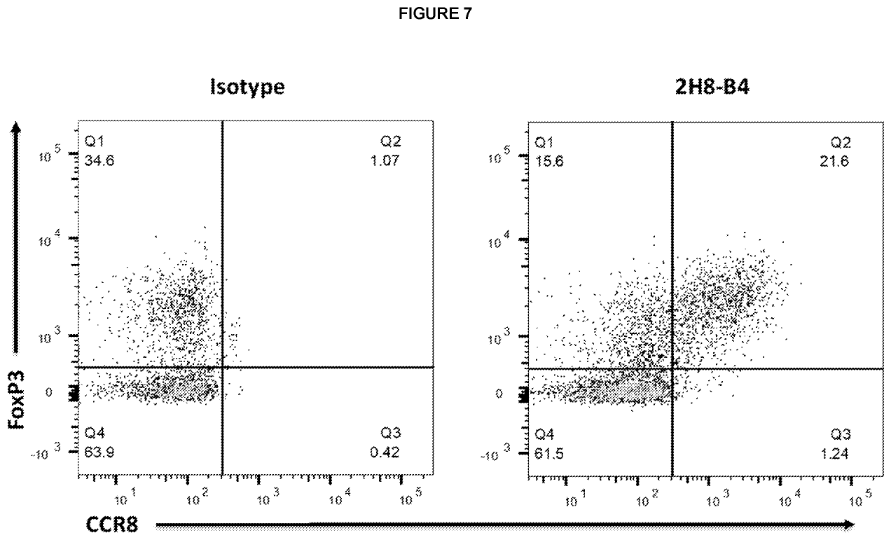

2H8 was found to bind to tumour infiltrating Tregs (CD3+; CD4+; FoxP3+ gate) while isotype control did not (FIG. 7).

### Example 9—Chimeric 2H8 does not Inhibit CCL1 Induced Chemotaxis

Next the inventor(s) performed a chemotaxis assay to determine if chimeric 2H8 would block CCL1 induced chemotaxis. It is known that CCL1 binds to the N-terminus of CCR8 to induce chemotaxis.

Briefly, L1.2 cells stably transfected with human CCR8 were maintained in suspension in RPMI 1640 containing 10% heat-inactivated FCS, 2% 1-glutamine and 100 U ml-1 penicillin. For the assay, the cells were washed once in PBS and resuspended at 106 cells ml-1 in assay buffer (RPMI 1640, 1% endotoxin-free BSA, 100 U ml-1 penicillin, and 100 μg ml-1 streptomycin). Antibodies were preincubated with 100 μl of cells (1×105 cells) for 30 min and placed into the upper chamber of a transwell plate with 5-μm pores (Corning Life Sciences). In the lower chambers, 5.8 nM of human CCL1 (30 min, 37° C.) was placed and the assay was incubated at 37° C. for 4 h (5% CO2). Live cells migrating to the lower chamber were counted using a LSRII flow cytometer.

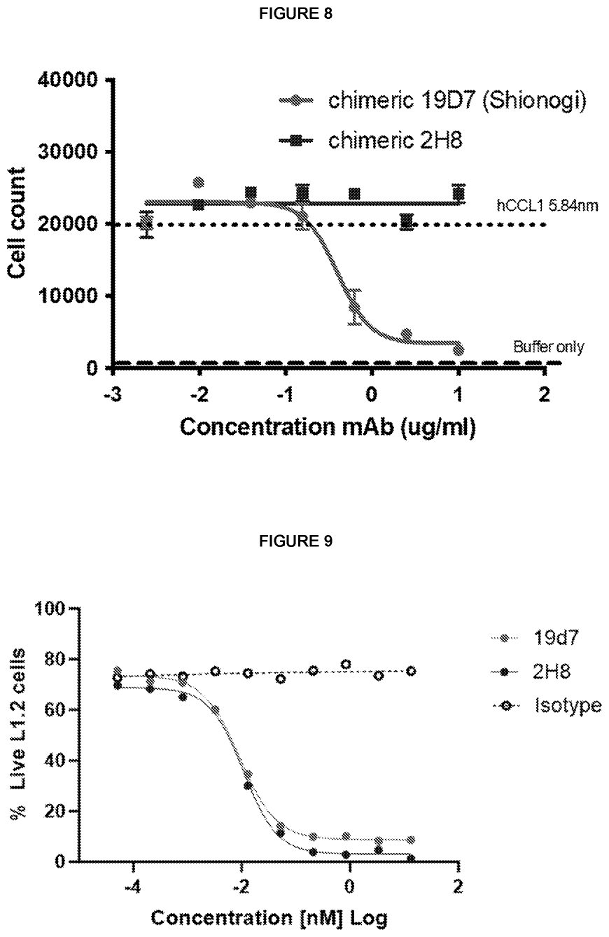

Chimeric 2H8 did not inhibit CCL1 induced chemotaxis, while chimeric 19D7 had a significant and dose-dependent inhibitory effect on CCL1 induced chemotaxis (FIG. 8). Advantageously, the inventors believe that in an environment with high CCL1 concentration, such as a tumour microenvironment, binding of chimeric 2H8 to CCR8 would not be inhibited by competition with CCL1 as they do not share the same binding site, unlike chimeric 19D7.

### Example 10—Epitope Mapping of 2H8

To further explore the epitope binding of 2H8, the inventor(s) conducted epitope mapping. Specifically, the inventor(s) replaced the N-terminus, extracellular loop 1, extracellular loop 2 and extracellular loop 3 in human CCR8 with the N-terminus, extracellular loop 1, extracellular loop 2 and extracellular loop 3 of human CCR5 respectively. These modified hCCR8 are shown in 5888, 8588, 8858 and 8885 in Table 3 respectfully with the amino acid sequence from CCR5 underlined. The results of the epitope mapping are shown in Table 3 below.

TABLE 3
Epitope mapping of 2H8 on chimeric transfected cells and hCCR8 2 ECL mutants

| | | | | | Mut | Mut | Mut | Mut |
|---|---|---|---|---|---|---|---|---|
| | 5888 | 8588 | 8858 | 8885 | 172-173 | 174-175 | 176-177 | 178-179 |
| | (SEQ ID | (SEQ ID | (SEQ ID | (SEQ ID | (SEQ ID | (SEQ ID | (SEQ ID | (SEQ ID |
| Antibody | NO: 75) | NO: 76) | NO: 77) | NO: 78) | NO: 79) | NO: 80) | NO: 81) | NO: 82) |
| Isotype | − | − | − | − | − | − | − | − |
| 2H8 | + | + | − | + | + | + | − | − |
| 19D7 | − | + | + | + | + | + | + | + |
| (Shionogi) | | | | | | | | |

Interestingly, mutation to the N-terminus of human CCR8 (5888 in Table 3) prevented binding of 19D7 to CCR8, but 2H8 was still able to bind to CCR8. Whereas, mutation to the extracellular loop 2 (8858 in Table 3) prevented binding of 2H8 to CCR8, while 19D7 was still able to bind to CCR8.

These results suggest that 2H8 and 19D7 bind to different epitopes on CCR8.

### Example 11—Chimeric 2H8 Exhibits ADCC Potential with Efficacy and Potency Comparable to 19D7

To further explore the functional properties of chimeric 2H8, the inventor(s) performed a dose-dependent antibody-dependent cellular cytotoxicity (ADCC) assay.

The ability of antibodies to induce ADCC was evaluated by flow cytometry. Briefly, hCCR8 L1.2 expressing cells were labeled with membrane dye, PKH26, to allow discrimination when incubated with effector cells and antibodies. Labeled target cells were washed 3 times with culture medium and resuspended in culture medium at a concentration of 1×10⁶/ml. Labeled target cells were dispensed in round-bottomed 96-well plates (1×10⁵ in 100 μl/well) and preincubated with 5 μg; 1 μg; 0.5 μg or 0.1 μg/ml of antibodies at 37° C. for 30 minutes. PBMCs were prepared from heparinized blood (obtained from healthy individuals) by centrifugation on a Ficoll gradient. Human natural killer cells were next isolated from the PBMCs using MACS CD56 microbeads and MACS positive selection columns (Miltenyi Biotec) in accordance with the manufacturer's protocol. Thereafter, 2×10⁵ purified human NK cells (effector cells), were incubated with the target cells/antibody mixture (E:T ratios=2:1) at 37° C. for 3 hours in the presence of 10% heat-inactivated serum. Treated cells were washed 3 times and resuspended in 200 μl of PBS. Just before analysis on a LSRII flow cytometer, TO-PRO 3 iodide was added to detect cell death. As a counting standard, 20 μl/well CountBright absolute counting beads (Invitrogen) were added to determine cell concentration of cell subsets. Samples were acquired on an LSRII flow cytometer.

Chimeric 2H8 and Shionogi's chimeric 19D7 anti-CCR8 antibodies induced similar levels of cellular cytotoxicity as shown in FIG. 9 and Table 4 below.

TABLE 4
2H8 induced cellular cytotoxicity in an ADCC assay

| | 19D7 | 2H8 | Isotype |
|---|---|---|---|
| IC50 | 0.009163 | 0.01067 | 5.87E−27 |
| R² | 0.9987 | .9976 | |

### Example 12—In Vivo Activity of 2H8 in a Colorectal Cancer Mouse Model

To test the activity of 2H8 in vivo, the inventor(s) subcutaneously implanted 5×10⁵ MC38 colorectal cells in C57BL/6 mice. Animals were treated with 2H8 mlgG2a (n=9), anti-mPD1 (n=10), and isotype control (n=6). Tumor volume (mm3) was measured daily with a digital calliper after the first injection of antibodies and compared between groups. Treatment was conducted 2×/week with a dose of 5 mg/kg for a maximum duration of 3 weeks.

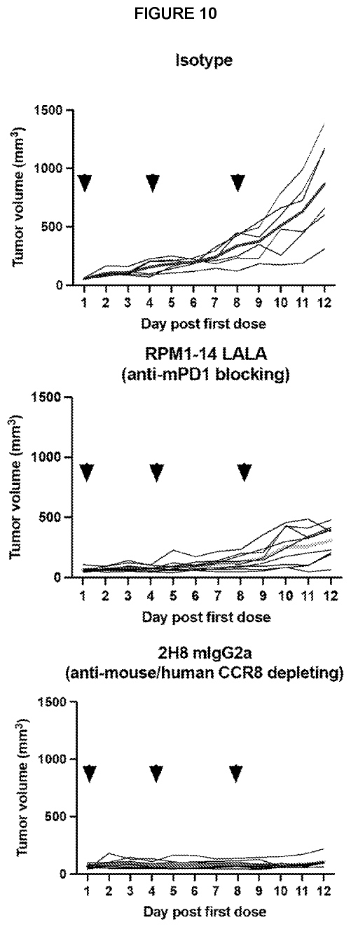

Treatment with 2H8 substantially inhibited tumour growth compared to both isotype control and anti-mPD1 treatment groups (FIG. 10).

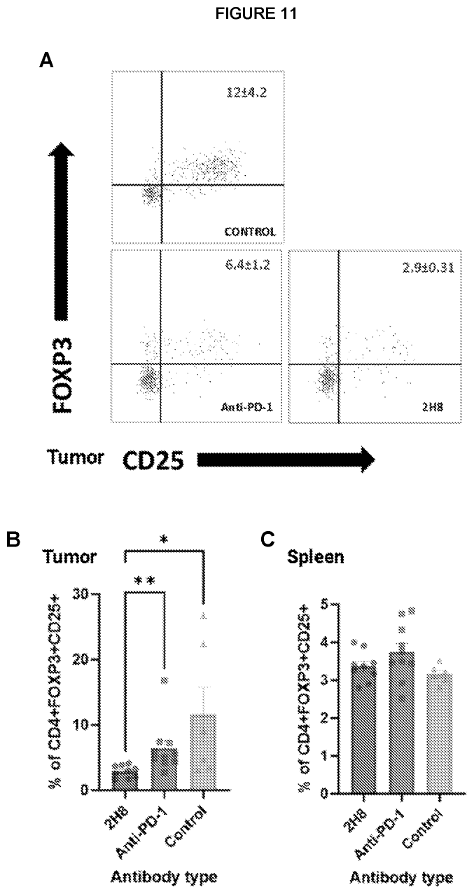

At the endpoint, tumor and spleen tissues were processed, stained, and analyzed on flow cytometry. The percentage of CD4⁺FOXP3⁺CD25⁺ cells in TCR-β⁺ cells (parent population) was collected and compared between the three groups. 2H8 treatment caused Tregs depletion in the tumor but not in the spleen (FIG. 11). Furthermore, Treg depletion was significantly greater in the 2H8 treatment group compared to anti-PD1 or isotype control.

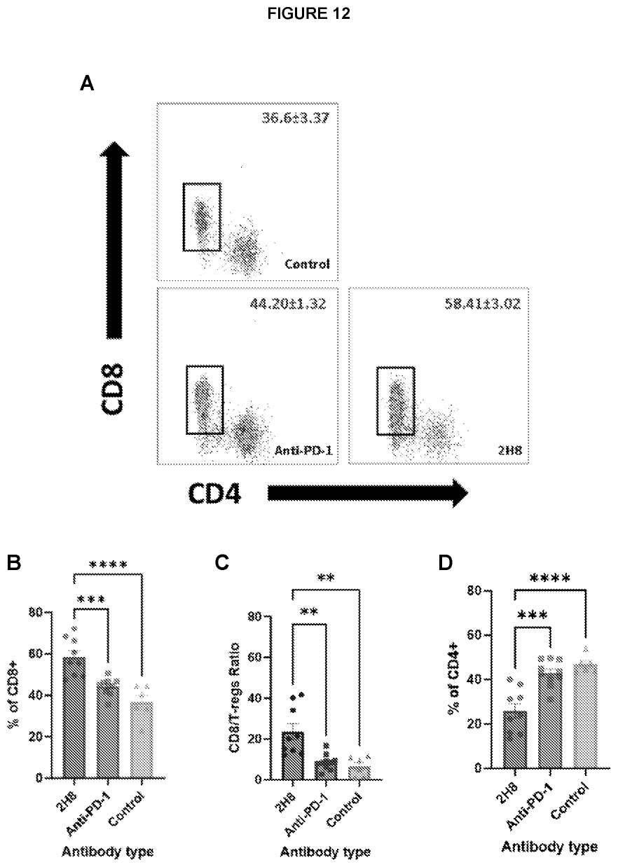

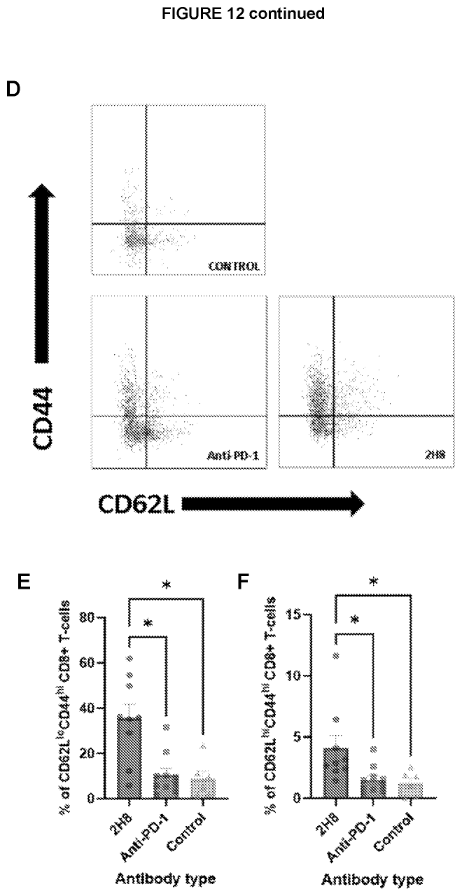

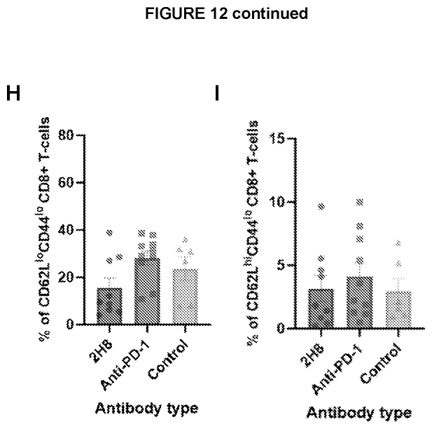

The inventor(s) next analysed CD8+ T cell populations and other CD4+ T cell populations in the tumour samples. The inventor(s) found that 2H8 treatment increased tumor-specific CD8+ T-cells and reduced CD4+ T-cells, which as statistically significant compared to anti-PD1 or isotype control (FIG. 12).

It will be understood that the invention disclosed and defined in this specification extends to all alternative combinations of two or more of the individual features mentioned or evident from the text or drawings. All of these different combinations constitute various alternative aspects of the invention.

1. An antigen binding protein that binds to or specifically binds to the extracellular loop 2 of CCR8.

2. The antigen binding protein of claim 1, wherein the antigen binding protein binds to residues 172 to 202 of CCR8, preferably residues 172 to 190 of CCR8, wherein the amino acid sequence of CCR8 is as set forth in SEQ ID NO: 55.

3. The antigen binding protein of claim 1 or 2, wherein the antigen binding protein binds to residues 176 to 179 of CCR8, wherein the amino acid sequence of CCR8 is as set forth in SEQ ID NO: 55.

4. The antigen binding protein of any one of claims 1 to 3, wherein the antigen binding protein does not block binding of CCR8 to CCL1.

5. An antigen binding protein comprising a CDRH1, a CDRH2 and/or a CDRH3 of an antibody having a variable heavy chain as defined in SEQ ID NO: 1 and a CDRL1, a CDRL2 and/or a CDRL3 of an antibody having a variable light chain as defined in SEQ ID NO: 2.

6. An antigen binding protein that binds to or specifically binds to CCR8 and wherein the antigen binding protein competitively inhibits the binding of an antibody comprising: a VH comprising a sequence as set forth in SEQ ID NO: 1 and a VL comprising a sequence as set forth in SEQ ID NO: 2; or a VH comprising a sequence as set forth in SEQ ID NO: 83 and a VL comprising a sequence as set forth in SEQ ID NO: 84.

7. The antigen binding protein of any one of claims 1 to 6, wherein the antigen binding protein comprises an antigen binding domain of an antibody, wherein the antigen binding domain binds to or specifically binds to CCR8, wherein the antigen binding domain comprises at least one of:
(i) a VH comprising a complementarity determining region (CDR) 1 comprising a sequence at least about 80%, at least 85%, at least 90%, at least 91% at least 92%, at least 93%, at least 94%, at least 95%, at least 96%, at least 97%, at least 98%, at least 99% identical to a sequence set forth in SEQ ID NO: 5, a CDR2 comprising a sequence at least about 80%, at least 85%, at least 90%, at least 92%, at least 95%, at least 97%, at least 99% identical to a sequence set in SEQ ID NO: 6, and a CDR3 comprising a sequence at least about 80%, at least 85%, at least 90%, at least 92%, at least 95%, at least 97%, at least 99% identical to a sequence set forth in SEQ ID NO: 7;
(ii) a VH comprising a sequence at least about 80%, at least 85%, at least 90%, at least 91% at least 92%, at least 93%, at least 94%, at least 95%, at least 96%, at least 97%, at least 98%, at least 99% identical to a sequence set forth in SEQ ID NO: 1 or 83;
(iii) a VL comprising a CDR1 comprising a sequence at least about 80%, at least 85%, at least 90%, at least 91% at least 92%, at least 93%, at least 94%, at least 95%, at least 96%, at least 97%, at least 98%, at least 99% identical to a sequence set forth in SEQ ID NO: 8, a CDR2 comprising a sequence at least about 80%, at least 85%, at least 90%, at least 91% at least 92%, at least 93%, at least 94%, at least 95%, at least 96%, at least 97%, at least 98%, at least 99% identical to a sequence set forth in SEQ ID NO: 9, and a CDR3 comprising a sequence at least about 80%, at least 85%, at least 90%, at least 91% at least 92%, at least 93%, at least 94%, at least 95%, at least 96%, at least 97%, at least 98%, at least 99% identical to a sequence set forth in SEQ ID NO: 10;
(iv) a VL comprising a sequence at least about 80%, at least 85%, at least 90%, at least 91% at least 92%, at least 93%, at least 94%, at least 95%, at least 96%, at least 97%, at least 98%, at least 99% identical to a sequence set forth in SEQ ID NO: 2 or 84;
(v) a VH comprising a CDR1 comprising a sequence set forth in SEQ ID NO: 5, a CDR2 comprising a sequence set forth between in SEQ ID NO: 6, and a CDR3 comprising a sequence set forth in SEQ ID NO: 7;
(vi) a VH comprising a sequence set forth in SEQ ID NO: 1 or 83;
(vii) a VL comprising a CDR1 comprising a sequence set SEQ ID NO: 8, a CDR2 comprising a sequence set forth in SEQ ID NO: 9 and a CDR3 comprising a sequence set forth in SEQ ID NO: 10;
(viii) a VL comprising a sequence set forth in SEQ ID NO: 2 or 84;
(ix) a VH comprising a CDR1 comprising a sequence set forth in SEQ ID NO: 5, a CDR2 comprising a sequence set forth between in SEQ ID NO: 6 and a CDR3 comprising a sequence set forth in SEQ ID NO: 7; and a VL comprising a CDR1 comprising a sequence set SEQ ID NO: 8, a CDR2 comprising a sequence set forth in SEQ ID NO: 9 and a CDR3 comprising a sequence set forth in SEQ ID NO: 10;
(x) a VH comprising a sequence set forth in SEQ ID NO: 1 and a VL comprising a sequence set forth in SEQ ID NO: 2,
(xi) a VH comprising a sequence set forth in SEQ ID NO: 83 and a VL comprising a sequence set forth in SEQ ID NO: 84.

8. The antigen binding domain of claim 7, further comprising at least one of:
(i) a VH comprising a framework region (FR) 1 comprising a sequence at least about 80%, at least 85%, at least 90%, at least 91% at least 92%, at least 93%, at least 94%, at least 95%, at least 96%, at least 97%, at least 98%, at least 99% identical to a sequence set forth in SEQ ID NO: 11, a FR2 comprising a sequence at least about 80%, at least 85%, at least 90%, at least 91% at least 92%, at least 93%, at least 94%, at least 95%, at least 96%, at least 97%, at least 98%, at least 99% identical to a sequence set in SEQ ID NO: 12, a FR3 comprising a sequence at least about 80%, at least 85%, at least 90%, at least 91% at least 92%, at least 93%, at least 94%, at least 95%, at least 96%, at least 97%, at least 98%, at least 99% identical to a sequence set forth in SEQ ID NO: 13, and a FR4 comprising a sequence at least about 80%, at least 85%, at least 90%, at least 91% at least 92%, at least 93%, at least 94%, at least 95%, at least 96%, at least 97%, at least 98%, at least 99% identical to a sequence set forth in SEQ ID NO: 14;
(ii) a VL comprising a FR1 comprising a sequence at least about 80%, at least 85%, at least 90%, at least 91% at least 92%, at least 93%, at least 94%, at least 95%, at least 96%, at least 97%, at least 98%, at least 99% identical to a sequence set forth in SEQ ID NO: 15, a FR2 comprising a sequence at least about 80%, at least 85%, at least 90%, at least 91% at least 92%, at least 93%, at least 94%, at least 95%, at least 96%, at least 97%, at least 98%, at least 99% identical to a sequence set forth in SEQ ID NO: 16, a FR3 comprising a sequence at least about 80%, at least 85%, at least 90%, at least 91% at least 92%, at least 93%, at least 94%, at least 95%, at least 96%, at least 97%, at least 98%, at least 99% identical to a sequence set forth in SEQ ID NO: 17, and a FR4 comprising a sequence at least about 80%, at least 85%, at least 90%, at least 91% at least 92%, at least 93%, at least 94%, at least 95%, at least 96%, at least 97%, at least 98%, at least 99% identical to a sequence set forth in SEQ ID NO: 18;
(iii) a VH comprising a FR1 comprising a sequence set forth in SEQ ID NO: 11, a FR2 comprising a sequence set forth between in SEQ ID NO: 12, a FR3 comprising a sequence set forth in SEQ ID NO: 13, and a FR4 comprising a sequence set forth in SEQ ID NO: 14;
(iv) a VL comprising a FR1 comprising a sequence set forth in SEQ ID NO: 15, a FR2 comprising a sequence set forth between in SEQ ID NO: 16, a FR3 comprising a sequence set forth in SEQ ID NO: 17, and a FR4 comprising a sequence set forth in SEQ ID NO: 18; or
(v) a VH comprising a FR1 comprising a sequence set forth in SEQ ID NO: 11, a FR2 comprising a sequence set forth between in SEQ ID NO: 12, a FR3 comprising a sequence set forth in SEQ ID NO: 13, and a FR4 comprising a sequence set forth in SEQ ID NO: 14; and a VL comprising a FR1 comprising a sequence set forth in SEQ ID NO: 15, a FR2 comprising a sequence set forth between in SEQ ID NO: 16, a FR3 comprising a sequence set forth in SEQ ID NO: 17, and a FR4 comprising a sequence set forth in SEQ ID NO: 18.

9. The antigen binding domain of claim 7, further comprising at least one of:
(i) a VH comprising a framework region (FR) 1 comprising a sequence at least about 80%, at least 85%, at least 90%, at least 91% at least 92%, at least 93%, at least 94%, at least 95%, at least 96%, at least 97%, at least 98%, at least 99% identical to a sequence set forth in SEQ ID NO: 60, a FR2 comprising a sequence at least about 80%, at least 85%, at least 90%, at least 91% at least 92%, at least 93%, at least 94%, at least 95%, at least 96%, at least 97%, at least 98%, at least 99% identical to a sequence set in SEQ ID NO: 61, a FR3 comprising a sequence at least about 80%, at least 85%, at least 90%, at least 91% at least 92%, at least 93%, at least 94%, at least 95%, at least 96%, at least 97%, at least 98%, at least 99% identical to a sequence set forth in SEQ ID NO: 62, and a FR4 comprising a sequence at least about 80%, at least 85%, at least 90%, at least 91% at least 92%, at least 93%, at least 94%, at least 95%, at least 96%, at least 97%, at least 98%, at least 99% identical to a sequence set forth in SEQ ID NO: 63;
(ii) a VL comprising a FR1 comprising a sequence at least about 80%, at least 85%, at least 90%, at least 91% at least 92%, at least 93%, at least 94%, at least 95%, at least 96%, at least 97%, at least 98%, at least 99% identical to a sequence set forth in SEQ ID NO: 64, a FR2 comprising a sequence at least about 80%, at least 85%, at least 90%, at least 91% at least 92%, at least 93%, at least 94%, at least 95%, at least 96%, at least 97%, at least 98%, at least 99% identical to a sequence set forth in SEQ ID NO: 65, a FR3 comprising a sequence at least about 80%, at least 85%, at least 90%, at least 91% at least 92%, at least 93%, at least 94%, at least 95%, at least 96%, at least 97%, at least 98%, at least 99% identical to a sequence set forth in SEQ ID NO: 66, and a FR4 comprising a sequence at least about 80%, at least 85%, at least 90%, at least 91% at least 92%, at least 93%, at least 94%, at least 95%, at least 96%, at least 97%, at least 98%, at least 99% identical to a sequence set forth in SEQ ID NO: 67;
(iii) a VH comprising a FR1 comprising a sequence set forth in SEQ ID NO: 60, a FR2 comprising a sequence set forth between in SEQ ID NO: 61, a FR3 comprising a sequence set forth in SEQ ID NO: 62, and a FR4 comprising a sequence set forth in SEQ ID NO: 63;
(iv) a VL comprising a FR1 comprising a sequence set forth in SEQ ID NO: 64, a FR2 comprising a sequence set forth between in SEQ ID NO: 65, a FR3 comprising a sequence set forth in SEQ ID NO: 66, and a FR4 comprising a sequence set forth in SEQ ID NO: 67; or
(v) a VH comprising a FR1 comprising a sequence set forth in SEQ ID NO: 60, a FR2 comprising a sequence set forth between in SEQ ID NO: 61, a FR3 comprising a sequence set forth in SEQ ID NO: 62, and a FR4 comprising a sequence set forth in SEQ ID NO: 63; and a VL comprising a FR1 comprising a sequence set forth in SEQ ID NO: 64, a FR2 comprising a sequence set forth between in SEQ ID NO: 65, a FR3 comprising a sequence set forth in SEQ ID NO: 66, and a FR4 comprising a sequence set forth in SEQ ID NO: 67.

10. The antigen binding protein of claim 5 or 6, wherein the antigen binding protein comprises an antigen binding domain of an antibody, wherein the antigen binding domain binds to or specifically binds to CCR8, wherein the antigen binding domain comprises at least one of:
(i) a VH comprising a complementarity determining region (CDR) 1 comprising a sequence at least about 80%, at least 85%, at least 90%, at least 91% at least 92%, at least 93%, at least 94%, at least 95%, at least 96%, at least 97%, at least 98%, at least 99% identical to a sequence set forth in SEQ ID NO: 19, a CDR2 comprising a sequence at least about 80%, at least 85%, at least 90%, at least 92%, at least 95%, at least 97%, at least 99% identical to a sequence set in SEQ ID NO: 20, and a CDR3 comprising a sequence at least about 80%, at least 85%, at least 90%, at least 92%, at least 95%, at least 97%, at least 99% identical to a sequence set forth in SEQ ID NO: 21;
(ii) a VH comprising a sequence at least about 80%, at least 85%, at least 90%, at least 91% at least 92%, at least 93%, at least 94%, at least 95%, at least 96%, at least 97%, at least 98%, at least 99% identical to a sequence set forth in SEQ ID NO: 1 or 83;
(iii) a VL comprising a CDR1 comprising a sequence at least about 80%, at least 85%, at least 90%, at least 91% at least 92%, at least 93%, at least 94%, at least 95%, at least 96%, at least 97%, at least 98%, at least 99% identical to a sequence set forth in SEQ ID NO: 22, a CDR2 comprising a sequence at least about 80%, at least 85%, at least 90%, at least 91% at least 92%, at least 93%, at least 94%, at least 95%, at least 96%, at least 97%, at least 98%, at least 99% identical to a sequence set forth in SEQ ID NO: 23, and a CDR3 comprising a sequence at least about 80%, at least 85%, at least 90%, at least 91% at least 92%, at least 93%, at least 94%, at least 95%, at least 96%, at least 97%, at least 98%, at least 99% identical to a sequence set forth in SEQ ID NO: 10;
(iv) a VL comprising a sequence at least about 80%, at least 85%, at least 90%, at least 91% at least 92%, at least 93%, at least 94%, at least 95%, at least 96%, at least 97%, at least 98%, at least 99% identical to a sequence set forth in SEQ ID NO: 2 or 84;
(v) a VH comprising a CDR1 comprising a sequence set forth in SEQ ID NO: 19, a CDR2 comprising a sequence set forth between in SEQ ID NO: 20, and a CDR3 comprising a sequence set forth in SEQ ID NO: 21;
(vi) a VH comprising a sequence set forth in SEQ ID NO: 1 or 83;
(vii) a VL comprising a CDR1 comprising a sequence set SEQ ID NO: 22, a CDR2 comprising a sequence set forth in SEQ ID NO: 23 and a CDR3 comprising a sequence set forth in SEQ ID NO: 10;
(viii) a VL comprising a sequence set forth in SEQ ID NO: 2 or 84;
(ix) a VH comprising a CDR1 comprising a sequence set forth in SEQ ID NO: 19, a CDR2 comprising a sequence set forth between in SEQ ID NO: 20 and a CDR3 comprising a sequence set forth in SEQ ID NO: 21; and a VL comprising a CDR1 comprising a sequence set SEQ ID NO: 22, a CDR2 comprising a sequence set forth in SEQ ID NO: 23 and a CDR3 comprising a sequence set forth in SEQ ID NO: 10;
(x) a VH comprising a sequence set forth in SEQ ID NO: 1 and a VL comprising a sequence set forth in SEQ ID NO: 2; or
(xi) a VH comprising a sequence set forth in SEQ ID NO: 83 and a VL comprising a sequence set forth in SEQ ID NO: 84.

11. The antigen binding domain of claim 10, further comprising at least one of:
(i) a VH comprising a framework region (FR) 1 comprising a sequence at least about 80%, at least 85%, at least 90%, at least 91% at least 92%, at least 93%, at least 94%, at least 95%, at least 96%, at least 97%, at least 98%, at least 99% identical to a sequence set forth in SEQ ID NO: 24, a FR2 comprising a sequence at least about 80%, at least 85%, at least 90%, at least 91% at least 92%, at least 93%, at least 94%, at least 95%, at least 96%, at least 97%, at least 98%, at least 99% identical to a sequence set in SEQ ID NO: 25, a FR3 comprising a sequence at least about 80%, at least 85%, at least 90%, at least 91% at least 92%, at least 93%, at least 94%, at least 95%, at least 96%, at least 97%, at least 98%, at least 99% identical to a sequence set forth in SEQ ID NO: 26, and a FR4 comprising a sequence at least about 80%, at least 85%, at least 90%, at least 91% at least 92%, at least 93%, at least 94%, at least 95%, at least 96%, at least 97%, at least 98%, at least 99% identical to a sequence set forth in SEQ ID NO: 14;
(ii) a VL comprising a FR1 comprising a sequence at least about 80%, at least 85%, at least 90%, at least 91% at least 92%, at least 93%, at least 94%, at least 95%, at least 96%, at least 97%, at least 98%, at least 99% identical to a sequence set forth in SEQ ID NO: 27, a FR2 comprising a sequence at least about 80%, at least 85%, at least 90%, at least 91% at least 92%, at least 93%, at least 94%, at least 95%, at least 96%, at least 97%, at least 98%, at least 99% identical to a sequence set forth in SEQ ID NO: 28, a FR3 comprising a sequence at least about 80%, at least 85%, at least 90%, at least 91% at least 92%, at least 93%, at least 94%, at least 95%, at least 96%, at least 97%, at least 98%, at least 99% identical to a sequence set forth in SEQ ID NO: 29, and a FR4 comprising a sequence at least about 80%, at least 85%, at least 90%, at least 91% at least 92%, at least 93%, at least 94%, at least 95%, at least 96%, at least 97%, at least 98%, at least 99% identical to a sequence set forth in SEQ ID NO: 18;
(iii) a VH comprising a FR1 comprising a sequence set forth in SEQ ID NO: 24, a FR2 comprising a sequence set forth between in SEQ ID NO: 25, a FR3 comprising a sequence set forth in SEQ ID NO: 26, and a FR4 comprising a sequence set forth in SEQ ID NO: 14;
(iv) a VL comprising a FR1 comprising a sequence set forth in SEQ ID NO: 27, a FR2 comprising a sequence set forth between in SEQ ID NO: 28, a FR3 comprising a sequence set forth in SEQ ID NO: 29, and a FR4 comprising a sequence set forth in SEQ ID NO: 18; or
(v) a VH comprising a FR1 comprising a sequence set forth in SEQ ID NO: 24, a FR2 comprising a sequence set forth between in SEQ ID NO: 25, a FR3 comprising a sequence set forth in SEQ ID NO: 26, and a FR4 comprising a sequence set forth in SEQ ID NO: 14; and a VL comprising a FR1 comprising a sequence set forth in SEQ ID NO: 27, a FR2 comprising a sequence set forth between in SEQ ID NO: 28, a FR3 comprising a sequence set forth in SEQ ID NO: 29, and a FR4 comprising a sequence set forth in SEQ ID NO: 18.

12. The antigen binding domain of claim 10, further comprising at least one of:
(i) a VH comprising a framework region (FR) 1 comprising a sequence at least about 80%, at least 85%, at least 90%, at least 91% at least 92%, at least 93%, at least 94%, at least 95%, at least 96%, at least 97%, at least 98%, at least 99% identical to a sequence set forth in SEQ ID NO: 68, a FR2 comprising a sequence at least about 80%, at least 85%, at least 90%, at least 91% at least 92%, at least 93%, at least 94%, at least 95%, at least 96%, at least 97%, at least 98%, at least 99% identical to a sequence set in SEQ ID NO: 69, a FR3 comprising a sequence at least about 80%, at least 85%, at least 90%, at least 91% at least 92%, at least 93%, at least 94%, at least 95%, at least 96%, at least 97%, at least 98%, at least 99% identical to a sequence set forth in SEQ ID NO: 70, and a FR4 comprising a sequence at least about 80%, at least 85%, at least 90%, at least 91% at least 92%, at least 93%, at least 94%, at least 95%, at least 96%, at least 97%, at least 98%, at least 99% identical to a sequence set forth in SEQ ID NO: 63;
(ii) a VL comprising a FR1 comprising a sequence at least about 80%, at least 85%, at least 90%, at least 91% at least 92%, at least 93%, at least 94%, at least 95%, at least 96%, at least 97%, at least 98%, at least 99% identical to a sequence set forth in SEQ ID NO: 71, a FR2 comprising a sequence at least about 80%, at least 85%, at least 90%, at least 91% at least 92%, at least 93%, at least 94%, at least 95%, at least 96%, at least 97%, at least 98%, at least 99% identical to a sequence set forth in SEQ ID NO: 72, a FR3 comprising a sequence at least about 80%, at least 85%, at least 90%, at least 91% at least 92%, at least 93%, at least 94%, at least 95%, at least 96%, at least 97%, at least 98%, at least 99% identical to a sequence set forth in SEQ ID NO: 73, and a FR4 comprising a sequence at least about 80%, at least 85%, at least 90%, at least 91% at least 92%, at least 93%, at least 94%, at least 95%, at least 96%, at least 97%, at least 98%, at least 99% identical to a sequence set forth in SEQ ID NO: 67;
(iii) a VH comprising a FR1 comprising a sequence set forth in SEQ ID NO: 68, a FR2 comprising a sequence set forth between in SEQ ID NO: 69, a FR3 comprising a sequence set forth in SEQ ID NO: 70, and a FR4 comprising a sequence set forth in SEQ ID NO: 63;
(iv) a VL comprising a FR1 comprising a sequence set forth in SEQ ID NO: 71, a FR2 comprising a sequence set forth between in SEQ ID NO: 72, a FR3 comprising a sequence set forth in SEQ ID NO: 73, and a FR4 comprising a sequence set forth in SEQ ID NO: 67; or
(v) a VH comprising a FR1 comprising a sequence set forth in SEQ ID NO: 68, a FR2 comprising a sequence set forth between in SEQ ID NO: 69, a FR3 comprising a sequence set forth in SEQ ID NO: 70, and a FR4 comprising a sequence set forth in SEQ ID NO: 63; and a VL comprising a FR1 comprising a sequence set forth in SEQ ID NO: 71, a FR2 comprising a sequence set forth between in SEQ ID NO: 72, a FR3 comprising a sequence set forth in SEQ ID NO: 73, and a FR4 comprising a sequence set forth in SEQ ID NO: 67

13. The antigen binding protein of any one of claims 1 to 12, wherein the antigen binding protein is in the form of:
(i) a single chain Fv fragment (scFv);
(ii) a dimeric scFv (di-scFv); or
(iii) one of (i) or (ii) linked to a constant region of an antibody, Fc or a heavy chain constant domain (CH) 2 and/or CH3.

14. The antigen binding protein of any one of claims 1 to 12, wherein the antigen binding protein is in the form of:
(i) a diabody;
(ii) a triabody;
(iii) a tetrabody;
(iv) a Fab;
(v) a F(ab′)2;
(vi) a Fv;
(vii) a bispecific antibody or other form of multispecific antibody; or
(viii) one of (i) to (vii) linked to a constant region of an antibody, Fc or a heavy chain constant domain (CH) 2 and/or CH3.

15. An anti-CCR8 antibody or antigen binding fragment thereof comprising a light chain variable region and a heavy chain variable region,
wherein said heavy chain variable region comprises:
a CDR H1 as set forth in SEQ ID NO: 5, a CDR H2 as set forth in SEQ ID NO: 6, and a CDR H3 as set forth in SEQ ID NO: 7; and
wherein said light chain variable region comprises:
a CDR L1 as set forth in SEQ ID NO: 8, a CDR L2 as set forth in SEQ ID NO: 9 and a CDR L3 as set forth in SEQ ID NO: 10.

16. The anti-CCR8 antibody or antigen binding fragment thereof of claim 15, wherein the anti-CCR8 antibody or antigen binding fragment thereof comprises a light chain variable region that comprises:
a FR L1 as set forth in SEQ ID NO: 15, an FR L2 as set forth in SEQ ID NO: 16, a FR L3 as set forth in SEQ ID NO: 17 and a FR L4 as set forth in SEQ ID NO: 18; or
a FR L1 as set forth in SEQ ID NO: 64, an FR L2 as set forth in SEQ ID NO: 65, a FR L3 as set forth in SEQ ID NO: 66 and a FR L4 as set forth in SEQ ID NO: 67.

17. The anti-CCR8 antibody or antigen binding fragment thereof of claim 15 or 16, wherein the anti-CCR8 antibody or antigen binding fragment thereof comprises a heavy chain variable region that comprises:
a FR H1 as set forth in SEQ ID NO: 11, FR H2 as set forth in SEQ ID NO: 12, a FR H3 as set forth in SEQ ID NO: 13 and a FR H4 as set forth in SEQ ID NO: 14; or
a FR H1 as set forth in SEQ ID NO: 60, FR H2 as set forth in SEQ ID NO: 61, a FR H3 as set forth in SEQ ID NO: 62 and a FR H4 as set forth in SEQ ID NO: 63.

18. An anti-CCR8 antibody or antigen binding fragment thereof, comprising a light chain variable region and a heavy chain variable region,
wherein said heavy chain variable region comprises:
a CDR H1 as set forth in SEQ ID NO: 19, a CDR H2 as set forth in SEQ ID NO: 20, and a CDR H3 as set forth in SEQ ID NO: 21; and
wherein said light chain variable region comprises:
a CDR L1 as set forth in SEQ ID NO: 22, a CDR L2 as set forth in SEQ ID NO: 23 and a CDR L3 as set forth in SEQ ID NO: 10.

19. The anti-CCR8 antibody or antigen binding fragment thereof of claim 18, wherein the anti-CCR8 antibody or antigen binding fragment thereof comprises a light chain variable region that comprises:
a FR L1 as set forth in SEQ ID NO: 27, an FR L2 as set forth in SEQ ID NO: 28, a FR L3 as set forth in SEQ ID NO: 29 and a FR L4 as set forth in SEQ ID NO: 18; or
a FR L1 as set forth in SEQ ID NO: 71, an FR L2 as set forth in SEQ ID NO: 72, a FR L3 as set forth in SEQ ID NO: 73 and a FR L4 as set forth in SEQ ID NO: 67.

20. The anti-CCR8 antibody or antigen binding fragment thereof of claim 18 or 19, wherein the anti-CCR8 antibody or antigen binding fragment thereof comprises a heavy chain variable region that comprises:
a FR H1 as set forth in SEQ ID NO: 24, FR H2 as set forth in SEQ ID NO: 25, a FR H3 as set forth in SEQ ID NO: 26 and a FR H4 as set forth in SEQ ID NO: 14, or
a FR H1 as set forth in SEQ ID NO: 68, FR H2 as set forth in SEQ ID NO: 69, a FR H3 as set forth in SEQ ID NO: 70 and a FR H4 as set forth in SEQ ID NO: 63.

21. The anti-CCR8 receptor antibody or antigen binding fragment thereof of any one of claims 15 to 20, wherein the anti-CCR8 receptor antibody or antigen binding fragment thereof comprises a light chain variable region that comprises the sequence of SEQ ID NO: 2.

22. The anti-CCR8 receptor antibody or antigen binding fragment thereof of any one of claims 15 to 21, wherein the anti-CCR8 antibody or antigen binding fragment thereof comprises a heavy chain variable region that comprises the sequence of SEQ ID NO: 1.

23. The anti-CCR8 receptor antibody or antigen binding fragment thereof of any one of claims 15 to 20, wherein the anti-CCR8 receptor antibody or antigen binding fragment thereof comprises a light chain variable region that comprises the sequence of SEQ ID NO: 84.

24. The anti-CCR8 receptor antibody or antigen binding fragment thereof of any one of claims 15 to 20 and 23, wherein the anti-CCR8 antibody or antigen binding fragment thereof comprises a heavy chain variable region that comprises the sequence of SEQ ID NO: 83.

25. The antigen binding protein, anti-CCR8 antibody or antigen binding fragment thereof of any one of claims 1 to 24, wherein the antigen binding protein or anti-CCR8 receptor antibody or antigen binding fragment thereof further comprises at most 15, preferably, 0, 1, 2, 3, 4, 5, 6, 7, 8, 9, or 10, and preferably having 0, 1, 2, 3, 4 or 5 amino acid insertions, deletions, substitutions or a combination thereof with respect to the amino acid sequence of the indicated SEQ ID NO.

26. The antigen binding protein, anti-CCR8 antibody or antigen binding fragment thereof of claim 25, wherein the amino acid insertions, deletions, substitutions or a combination thereof are not in the CDRs.

27. The antigen binding protein of any one of claims 1 to 26, comprising human constant regions.

28. The antigen binding protein of claim 27, wherein the human constant regions comprise the amino acid sequence as shown in SEQ ID Nos: 58 and/or 59.

29. A nucleic acid encoding an antigen binding protein, antibody or antigen binding fragment thereof of any one of claims 1 to 28.

30. A vector comprising a nucleic acid of claim 29.

31. A cell comprising a vector of claim 30 or nucleic acid of claim 29.

32. A pharmaceutical composition comprising an antigen binding protein of any one of claims 1 to 8 or 17 to 20, antibody or antigen binding fragment thereof of any one of claims 9 to 20, nucleic acid of claim 21, vector of claim 22 or cell of claim 23 and a pharmaceutically acceptable carrier, diluent or excipient.

33. A kit or article of manufacture comprising an antigen binding protein of any one of claims 1 to 14 or 25 to 28, antibody or antigen binding fragment thereof of any one of claims 15 to 28, nucleic acid of claim 29, vector of claim 30, cell of claim 31, or pharmaceutical composition of claim 32.

34. A method for producing an antigen binding protein of any one of claims 1 to 14 or 25 to 28, antibody or antigen binding fragment of any one of claims 15 to 28 comprising expressing a nucleic acid of claim 29 a cell or non-human animal.

35. A method for the prevention or treatment a condition or disease associated with expression of CCR8 in an individual comprising the step of providing an antigen binding protein of any one of claims 1 to 14 or 25 to 28, antibody or antigen binding fragment thereof of any one of claims 15 to 28, nucleic acid of claim 29, vector of claim 30, cell of claim 31, or pharmaceutical composition of claim 32 to an individual requiring treatment for said condition or disease.

36. The method of claim 35, wherein the disease or condition associated with expression of CCR8.

37. The method of claim 35 or 36, wherein the disease or condition is cancer.

38. Use of an antigen binding protein of any one of claims 1 to 14 or 25 to 28, antibody or antigen binding fragment thereof of any one of claims 15 to 28, nucleic acid of claim 29, vector of claim 30, cell of claim 31, or pharmaceutical composition of claim 32 in the manufacture of a medicament for the treatment or prevention of cancer in a subject.

39. An antigen binding protein of any one of claims 1 to 14 or 25 to 28, antibody or antigen binding fragment thereof of any one of claims 15 to 28, nucleic acid of claim 29, vector of claim 30, cell of claim 31, or pharmaceutical composition of claim 32 for use in the treatment or prevention of cancer in a subject.


SEQUENCE LISTING (84 sequences)

SEQ ID NO: 1 - protein, 118 aa, Synthetic construct
EVQLQQSGPELVKPGASVKMSCKASGYTFTDYNMHWVKQSHGKSLEWIGYVNPNNGGTSYNQKFKGKATLTVNKSSSTAYMELRSLTSEDSAVYYCARWVLRRNFAYWGQGTLVTVSA

SEQ ID NO: 2 - protein, 107 aa, Synthetic construct
DIVMTQSHKFMSTSVGDRVSITCKASQDVNTAVAWYQQKPGQSPKLLIYWASTRHTGVPDRFTGSGSGTDYTLTISSVQAEDLALYYCQQHYRNPYTFGGGTKLEIK

SEQ ID NO: 3 - protein, 354 aa, Synthetic construct
GAGGTCCAGCTGCAACAGTCTGGACCTGAGCTGGTGAAGCCTGGGGCTTCAGTGAAGATGTCCTGCAAGGCTTCTGGATACACATTCACTGACTACAACATGCACTGGGTGAAGCAAAGCCATGGAAAGAGCCTTGAGTGGATTGGATATGTTAACCCTAACAATGGTGGTACTAGCTACAACCAGAAGTTCAAGGGCAAGGCCACATTGACTGTAAACAAGTCCTCCAGCACAGCCTACATGGAGCTCCGCAGCCTGACATCGGAGGATTCTGCAGTCTATTACTGTGCAAGATGGGTATTACGACGGAACTTTGCTTACTGGGGCCAAGGGACTCTGGTCACTGTCTCTGCA

SEQ ID NO: 4 - protein, 321 aa, Synthetic construct
GACATTGTGATGACCCAGTCTCACAAATTCATGTCCACATCAGTAGGAGACAGGGTCAGCATCACCTGCAAGGCCAGTCAGGATGTGAATACTGCTGTAGCCTGGTATCAACAAAAACCAGGGCAATCTCCTAAACTACTGATTTACTGGGCATCCACCCGGCACACTGGAGTCCCTGATCGCTTCACAGGCAGTGGATCTGGGACAGATTATACTCTCACCATCAGCAGTGTGCAGGCTGAAGACCTGGCACTTTATTACTGTCAGCAACATTATAGAAATCCGTACACGTTCGGAGGGGGGACCAAGCTGGAAATAAAA

SEQ ID NO: 5 - protein, 8 aa, Synthetic construct
GYTFTDYN

SEQ ID NO: 6 - protein, 8 aa, Synthetic construct
VNPNNGGT

SEQ ID NO: 7 - protein, 11 aa, Synthetic construct
ARWVLRRNFAY

SEQ ID NO: 8 - protein, 6 aa, Synthetic construct
QDVNTA

SEQ ID NO: 9 - skipped

SEQ ID NO: 10 - protein, 9 aa, Synthetic construct
QQHYRNPYT

SEQ ID NO: 11 - protein, 25 aa, Synthetic construct
EVQLQQSGPELVKPGASVKMSCKAS

SEQ ID NO: 12 - protein, 17 aa, Synthetic construct
MHWVKQSHGKSLEWIGY

SEQ ID NO: 13 - protein, 38 aa, Synthetic construct
SYNQKFKGKATLTVNKSSSTAYMELRSLTSEDSAVYYC

SEQ ID NO: 14 - protein, 11 aa, Synthetic construct
WGQGTLVTVSA

SEQ ID NO: 15 - protein, 26 aa, Synthetic construct
DIVMTQSHKFMSTSVGDRVSITCKAS

SEQ ID NO: 16 - protein, 17 aa, Synthetic construct
VAWYQQKPGQSPKLLIY

SEQ ID NO: 17 - protein, 36 aa, Synthetic construct
TRHTGVPDRFTGSGSGTDYTLTISSVQAEDLALYYC

SEQ ID NO: 18 - protein, 10 aa, Synthetic construct
FGGGTKLEIK

SEQ ID NO: 19 - protein, 5 aa, Synthetic construct
DYNMH

SEQ ID NO: 20 - protein, 17 aa, Synthetic construct
YVNPNNGGTSYNQKFKG

SEQ ID NO: 21 - protein, 9 aa, Synthetic construct
WVLRRNFAY

SEQ ID NO: 22 - protein, 11 aa, Synthetic construct
KASQDVNTAVA

SEQ ID NO: 23 - protein, 7 aa, Synthetic construct
WASTRHT

SEQ ID NO: 24 - protein, 30 aa, Synthetic construct
EVQLQQSGPELVKPGASVKMSCKASGYTFT

SEQ ID NO: 25 - protein, 14 aa, Synthetic construct
WVKQSHGKSLEWIG

SEQ ID NO: 26 - protein, 32 aa, Synthetic construct
KATLTVNKSSSTAYMELRSLTSEDSAVYYCAR

SEQ ID NO: 27 - protein, 23 aa, Synthetic construct
DIVMTQSHKFMSTSVGDRVSITC

SEQ ID NO: 28 - protein, 15 aa, Synthetic construct
WYQQKPGQSPKLLIY

SEQ ID NO: 29 - protein, 32 aa, Synthetic construct
GVPDRFTGSGSGTDYTLTISSVQAEDLALYYC

SEQ ID NO: 30 - DNA, 24 bp, Synthetic construct
  source 1..24: mol_type = other DNA
ggatacacattcactgactacaac

SEQ ID NO: 31 - DNA, 24 bp, Synthetic construct
  source 1..24: mol_type = other DNA
gttaaccctaacaatggtggtact

SEQ ID NO: 32 - DNA, 33 bp, Synthetic construct
  source 1..33: mol_type = other DNA
gcaagatgggtattacgacggaactttgcttac

SEQ ID NO: 33 - DNA, 18 bp, Synthetic construct
  source 1..18: mol_type = other DNA
caggatgtgaatactgct

SEQ ID NO: 34 - skipped

SEQ ID NO: 35 - DNA, 27 bp, Synthetic construct
  source 1..27: mol_type = other DNA
cagcaacattatagaaatccgtacacg

SEQ ID NO: 36 - DNA, 75 bp, Synthetic construct
  source 1..75: mol_type = other DNA
gaggtccagctgcaacagtctggacctgagctggtgaagcctggggcttcagtgaagatgtcctgcaaggcttct

SEQ ID NO: 37 - DNA, 51 bp, Synthetic construct
  source 1..51: mol_type = other DNA
atgcactgggtgaagcaaagccatggaaagagccttgagtggattggatat

SEQ ID NO: 38 - DNA, 114 bp, Synthetic construct
  source 1..114: mol_type = other DNA
agctacaaccagaagttcaagggcaaggccacattgactgtaaacaagtcctccagcacagcctacatggagctccgcagcctgacatcggaggattctgcagtctattactgt

SEQ ID NO: 39 - DNA, 33 bp, Synthetic construct
  source 1..33: mol_type = other DNA
tggggccaagggactctggtcactgtctctgca

SEQ ID NO: 40 - DNA, 78 bp, Synthetic construct
  source 1..78: mol_type = other DNA
gacattgtgatgacccagtctcacaaattcatgtccacatcagtaggagacagggtcagcatcacctgcaaggccagt

SEQ ID NO: 41 - DNA, 51 bp, Synthetic construct
  source 1..51: mol_type = other DNA
gtagcctggtatcaacaaaaaccagggcaatctcctaaactactgatttac

SEQ ID NO: 42 - DNA, 108 bp, Synthetic construct
  source 1..108: mol_type = other DNA
acccggcacactggagtccctgatcgcttcacaggcagtggatctgggacagattatactctcaccatcagcagtgtgcaggctgaagacctggcactttattactgt

SEQ ID NO: 43 - DNA, 30 bp, Synthetic construct
  source 1..30: mol_type = other DNA
ttcggaggggggaccaagctggaaataaaa

SEQ ID NO: 44 - DNA, 15 bp, Synthetic construct
  source 1..15: mol_type = other DNA
gactacaacatgcac

SEQ ID NO: 45 - DNA, 51 bp, Synthetic construct
  source 1..51: mol_type = other DNA
tatgttaaccctaacaatggtggtactagctacaaccagaagttcaagggc

SEQ ID NO: 46 - DNA, 27 bp, Synthetic construct
  source 1..27: mol_type = other DNA
tgggtattacgacggaactttgcttac

SEQ ID NO: 47 - DNA, 33 bp, Synthetic construct
  source 1..33: mol_type = other DNA
aaggccagtcaggatgtgaatactgctgtagcc

SEQ ID NO: 48 - DNA, 21 bp, synthetic construct
  source 1..21: mol_type = other DNA
tgggcatccacccggcacact

SEQ ID NO: 49 - DNA, 90 bp, Synthetic construct
  source 1..90: mol_type = other DNA
gaggtccagctgcaacagtctggacctgagctggtgaagcctggggcttcagtgaagatgtcctgcaaggcttctggatacacattcact

SEQ ID NO: 50 - DNA, 42 bp, Synthetic construct
  source 1..42: mol_type = other DNA
tgggtgaagcaaagccatggaaagagccttgagtggattgga

SEQ ID NO: 51 - DNA, 96 bp, Synthetic construct
  source 1..96: mol_type = other DNA
aaggccacattgactgtaaacaagtcctccagcacagcctacatggagctccgcagcctgacatcggaggattctgcagtctattactgtgcaaga

SEQ ID NO: 52 - DNA, 69 bp, Synthetic construct
  source 1..69: mol_type = other DNA
gacattgtgatgacccagtctcacaaattcatgtccacatcagtaggagacagggtcagcatcacctgc

SEQ ID NO: 53 - DNA, 45 bp, Synthetic construct
  source 1..45: mol_type = other DNA
tggtatcaacaaaaaccagggcaatctcctaaactactgatttac

SEQ ID NO: 54 - DNA, 96 bp, Synthetic construct
  source 1..96: mol_type = other DNA
ggagtccctgatcgcttcacaggcagtggatctgggacagattatactctcaccatcagcagtgtgcaggctgaagacctggcactttattactgt

SEQ ID NO: 55 - protein, 355 aa, Homo sapiens
MDYTLDLSVTTVTDYYYPDIFSSPCDAELIQTNGKLLLAVFYCLLFVFSLLGNSLVILVLVVCKKLRSITDVYLLNLALSDLLFVFSFPFQTYYLLDQWVFGTVMCKVVSGFYYIGFYSSMFFITLMSVDRYLAVVHAVYALKVRTIRMGTTLCLAVWLTAIMATIPLLVFYQVASEDGVLQCYSFYNQQTLKWKIFTNFKMNILGLLIPFTIFMFCYIKILHQLKRCQNHNKTKAIRLVLIVVIASLLFWVPFNVVLFLTSLHSMHILDGCSISQQLTYATHVTEIISFTHCCVNPVIYAFVGEKFKKHLSEIFQKSCSQIFNYLGRQMPRESCEKSSSCQQHSSRSSSVDYIL

SEQ ID NO: 56 - protein, 353 aa, Mus musculus
MDYTMEPNVTMTDYYPDFFTAPCDAEFLLRGSMLYLAILYCVLFVLGLLGNSLVILVLVGCKKLRSITDIYLLNLAASDLLFVLSIPFQTHNLLDQWVFGTAMCKVVSGLYYIGFFSSMFFITLMSVDRYLAIVHAVYAIKVRTASVGTALSLTVWLAAVTATIPLMVFYQVASEDGMLQCFQFYEEQSLRWKLFTHFEINALGLLLPFAILLFCYVRILQQLRGCLNHNRTRAIKLVLTVVIVSLLFWVPFNVALFLTSLHDLHILDGCATRQRLALAIHVTEVISFTHCCVNPVIYAFIGEKFKKHLMDVFQKSCSHIFLYLGRQMPVGALERQLSSNQRSSHSSTLDDIL

SEQ ID NO: 57 - protein, 355 aa, Macaca fascicularis
MDYTLDPSMTTMTDYYYPDSLSSPCDGELIQRNDKLLLAVFYCLLFVFSLLGNSLVILVLVVCKKLRNITDIYLLNLALSDLLFVFSFPFQTYYQLDQWVFGTVMCKVVSGFYYIGFYSSMFFITLMSVDRYLAVVHAVYAIKVRTIRMGTTLSLVVWLTAIMATIPLLVFYQVASEDGVLQCYSFYNQQTLKWKIFTNFEMNILGLLIPFTIFMFCYIKILHQLKRCQNHNKTKAIRLVLIVVIASLLFWVPFNVVLFLTSLHSMHILDGCSISQQLNYATHVTEIISFTHCCVNPVIYAFVGEKFKKHLSEIFQKSCSHIFIYLGRQMPRESCEKSSSCQQHSFRSSSIDYIL

SEQ ID NO: 58 - protein, 330 aa, Synthetic construct [sequence omitted]

SEQ ID NO: 59 - protein, 107 aa, Synthetic construct [sequence omitted]

SEQ ID NO: 60 - protein, 25 aa, Synthetic construct [sequence omitted]

SEQ ID NO: 61 - protein, 17 aa, Synthetic construct
MHWVRQAPGQGLEWIGY

SEQ ID NO: 62 - protein, 38 aa, Synthetic construct
SYNQKFKGKATLTVDKSISTAYMELSRLRSDDTAVYYC

SEQ ID NO: 63 - protein, 11 aa, Synthetic construct
WGQGTLVTVSS

SEQ ID NO: 64 - protein, 26 aa, Synthetic construct [sequence omitted]

SEQ ID NO: 65 - protein, 17 aa, Synthetic construct
VAWYQQKPGKAPKLLIY

SEQ ID NO: 66 - protein, 36 aa, Synthetic construct
TRHTGVPSRFSGSGSGTDFTLTISSLQPEDFATYYC

SEQ ID NO: 67 - protein, 10 aa, Synthetic construct
FGQGTKLEIK

SEQ ID NO: 68 - protein, 30 aa, Synthetic construct
QVQLVQSGAEVKKPGASVKVSCKASGYTFT

SEQ ID NO: 69 - protein, 14 aa, Synthetic construct
WVRQAPGQGLEWIG

SEQ ID NO: 70 - protein, 32 aa, Synthetic construct
KATLTVDKSISTAYMELSRLRSDDTAVYYCAR

SEQ ID NO: 71 - protein, 23 aa, Synthetic construct
DIQMTQSPSSLSASVGDRVTITC

SEQ ID NO: 72 - protein, 15 aa, Synthetic construct
WYQQKPGKAPKLLIY

SEQ ID NO: 73 - protein, 32 aa, Synthetic construct
GVPSRFSGSGSGTDFTLTISSLQPEDFATYYC

SEQ ID NO: 74 - protein, 31 aa, Homo sapiens
YQVASEDGVLQCYSFYNQQTLKWKIFTNFKM

SEQ ID NO: 75 - protein, 351 aa, Synthetic construct
MDYQVSSPIYDINYYTSEPCQKINVKQIAARLLLAVFYCLLFVFSLLGNSLVILVLVVCKKLRSITDVYLLNLALSDLLFVFSFPFQTYYLLDQWVFGTVMCKVVSGFYYIGFYSSMFFITLMSVDRYLAVVHAVYALKVRTIRMGTTLCLAVWLTAIMATIPLLVFYQVASEDGVLQCYSFYNQQTLKWKIFTNFKMNILGLLIPFTIFMFCYIKILHQLKRCQNHNKTKAIRLVLIVVIASLLFWVPFNVVLFLTSLHSMHILDGCSISQQLTYATHVTEIISFTHCCVNPVIYAFVGEKFKKHLSEIFQKSCSQIFNYLGRQMPRESCEKSSSCQQHSSRSSSVDYIL

SEQ ID NO: 76 - protein, 355 aa, Synthetic construct
MDYTLDLSVTTVTDYYYPDIFSSPCDAELIQTNGKLLLAVFYCLLFVFSLLGNSLVILVLVVCKKLRSITDVYLLNLALSDLLFVFSFPFQTYYAAAQWDFGNTMCQVVSGFYYIGFYSSMFFITLMSVDRYLAVVHAVYALKVRTIRMGTTLCLAVWLTAIMATIPLLVFYQVASEDGVLQCYSFYNQQTLKWKIFTNFKMNILGLLIPFTIFMFCYIKILHQLKRCQNHNKTKAIRLVLIVVIASLLFWVPFNVVLFLTSLHSMHILDGCSISQQLTYATHVTEIISFTHCCVNPVIYAFVGEKFKKHLSEIFQKSCSQIFNYLGRQMPRESCEKSSSCQQHSSRSSSVDYIL

SEQ ID NO: 77 - protein, 356 aa, Synthetic construct
MDYTLDLSVTTVTDYYYPDIFSSPCDAELIQTNGKLLLAVFYCLLFVFSLLGNSLVILVLVVCKKLRSITDVYLLNLALSDLLFVFSFPFQTYYLLDQWVFGTVMCKVVSGFYYIGFYSSMFFITLMSVDRYLAVVHAVYALKVRTIRMGTTLCLAVWLTAIMATIPLLVFTRSQKEGLHYTCSSHFPYSQYQFWKNFQTLKINILGLLIPFTIFMFCYIKILHQLKRCQNHNKTKAIRLVLIVVIASLLFWVPFNVVLFLTSLHSMHILDGCSISQQLTYATHVTEIISFTHCCVNPVIYAFVGEKFKKHLSEIFQKSCSQIFNYLGRQMPRESCEKSSSCQQHSSRSSSVDYIL

SEQ ID NO: 78 - protein, 355 aa, Synthetic construct
MDYTLDLSVTTVTDYYYPDIFSSPCDAELIQTNGKLLLAVFYCLLFVFSLLGNSLVILVLVVCKKLRSITDVYLLNLALSDLLFVFSFPFQTYYLLDQWVFGTVMCKVVSGFYYIGFYSSMFFITLMSVDRYLAVVHAVYALKVRTIRMGTTLCLAVWLTAIMATIPLLVFYQVASEDGVLQCYSFYNQQTLKWKIFTNFKMNILGLLIPFTIFMFCYIKILHQLKRCQNHNKTKAIRLVLIVVIASLLFWVPFNVVLFLTSLQEFFGLNNCSSSNRLDQATHVTEIISFTHCCVNPVIYAFVGEKFKKHLSEIFQKSCSQIFNYLGRQMPRESCEKSSSCQQHSSRSSSVDYIL

SEQ ID NO: 79 - protein, 355 aa, Synthetic construct
MDYTLDLSVTTVTDYYYPDIFSSPCDAELIQTNGKLLLAVFYCLLFVFSLLGNSLVILVLVVCKKLRSITDVYLLNLALSDLLFVFSFPFQTYYLLDQWVFGTVMCKVVSGFYYIGFYSSMFFITLMSVDRYLAVVHAVYALKVRTIRMGTTLCLAVWLTAIMATIPLLVFAAVASEDGVLQCYSFYNQQTLKWKIFTNFKMNILGLLIPFTIFMFCYIKILHQLKRCQNHNKTKAIRLVLIVVIASLLFWVPFNVVLFLTSLHSMHILDGCSISQQLTYATHVTEIISFTHCCVNPVIYAFVGEKFKKHLSEIFQKSCSQIFNYLGRQMPRESCEKSSSCQQHSSRSSSVDYIL

SEQ ID NO: 80 - protein, 355 aa, Synthetic construct
MDYTLDLSVTTVTDYYYPDIFSSPCDAELIQTNGKLLLAVFYCLLFVFSLLGNSLVILVLVVCKKLRSITDVYLLNLALSDLLFVFSFPFQTYYLLDQWVFGTVMCKVVSGFYYIGFYSSMFFITLMSVDRYLAVVHAVYALKVRTIRMGTTLCLAVWLTAIMATIPLLVFYQAGSEDGVLQCYSFYNQQTLKWKIFTNFKMNILGLLIPFTIFMFCYIKILHQLKRCQNHNKTKAIRLVLIVVIASLLFWVPFNVVLFLTSLHSMHILDGCSISQQLTYATHVTEIISFTHCCVNPVIYAFVGEKFKKHLSEIFQKSCSQIFNYLGRQMPRESCEKSSSCQQHSSRSSSVDYIL

SEQ ID NO: 81 - protein, 355 aa, Synthetic construct
MDYTLDLSVTTVTDYYYPDIFSSPCDAELIQTNGKLLLAVFYCLLFVFSLLGNSLVILVLVVCKKLRSITDVYLLNLALSDLLFVFSFPFQTYYLLDQWVFGTVMCKVVSGFYYIGFYSSMFFITLMSVDRYLAVVHAVYALKVRTIRMGTTLCLAVWLTAIMATIPLLVFYQVAAADGVLQCYSFYNQQTLKWKIFTNFKMNILGLLIPFTIFMFCYIKILHQLKRCQNHNKTKAIRLVLIVVIASLLFWVPFNVVLFLTSLHSMHILDGCSISQQLTYATHVTEIISFTHCCVNPVIYAFVGEKFKKHLSEIFQKSCSQIFNYLGRQMPRESCEKSSSCQQHSSRSSSVDYIL

SEQ ID NO: 82 - protein, 355 aa, Synthetic construct
MDYTLDLSVTTVTDYYYPDIFSSPCDAELIQTNGKLLLAVFYCLLFVFSLLGNSLVILVLVVCKKLRSITDVYLLNLALSDLLFVFSFPFQTYYLLDQWVFGTVMCKVVSGFYYIGFYSSMFFITLMSVDRYLAVVHAVYALKVRTIRMGTTLCLAVWLTAIMATIPLLVFYQVASEAAVLQCYSFYNQQTLKWKIFTNFKMNILGLLIPFTIFMFCYIKILHQLKRCQNHNKTKAIRLVLIVVIASLLFWVPFNVVLFLTSLHSMHILDGCSISQQLTYATHVTEIISFTHCCVNPVIYAFVGEKFKKHLSEIFQKSCSQIFNYLGRQMPRESCEKSSSCQQHSSRSSSVDYIL

SEQ ID NO: 83 - protein, 118 aa, Synthetic construct
QVQLVQSGAEVKKPGASVKVSCKASGYTFTDYNMHWVRQAPGQGLEWIGYVNPNNGGTSYNQKFKGKATLTVDKSISTAYMELSRLRSDDTAVYYCARWVLRRNFAYWGQGTLVTVSS

SEQ ID NO: 84 - protein, 107 aa, Synthetic construct
DIQMTQSPSSLSASVGDRVTITCKASQDVNTAVAWYQQKPGKAPKLLIYWASTRHTGVPSRFSGSGSGTDFTLTISSLQPEDFATYYCQQHYRNPYTFGQGTKLEIK